(19) United States
(12) Patent Application Publication
(10) Pub. No.: US 2018/0194836 A1
(43) Pub. Date: Jul. 12, 2018

(54) PREVENTION OF METASTASIS AND RECURRENCE AFTER PRIMARY CANCER TREATMENT

(71) Applicant: ENCYT TECHNOLOGIES, INC., Membertou (CA)

(72) Inventor: William Warren HARLESS, Middle River (CA)

(21) Appl. No.: 15/539,027

(22) PCT Filed: Dec. 22, 2015
(86) PCT No.: PCT/CA2015/000607

§ 371 Date: Jun. 22, 2017

Related U.S. Application Data
(60) Provisional application No. 62/212,368, filed on Aug. 31, 2015.
(60) Provisional application No. 62/180,945, filed on Jun. 17, 2015.
(60) Provisional application No. 62/180,925, filed on Jun. 17, 2015.
(60) Provisional application No. 62/095,515, filed on Dec. 22, 2014.

(51) Int. Cl. C07K 16/24; A61K 39/395; A61K 31/727; A61K 31/215; A61K 45/06; G01N 33/68; G01N 33/574

(52) U.S. Cl. CPC C07K 16/24; A61K 39/3955; A61K 31/727; A61K 2300/00; A61K 45/06; G01N 33/6863; G01N 33/574; A61K 31/215

(57) ABSTRACT

There is provided methods for inhibiting metastasis or recurrence of a cancer in a patient after a primary treatment of the patient, and compositions for same. The method comprises administering a therapeutically effective amount of a composition for inhibiting cancer stem cell enrichment In other embodiments, the method comprises administering a therapeutically effective amount of a cancer therapy targeted towards a population of proliferating cancer stem cells in the patient.

This application claims the benefit of U.S. Provisional Application No. 62/095,515 filed Dec. 22, 2014 and U.S. Provisional Applications Nos. 62/180,925 and 62/180,945 filed Jun. 17, 2015.

### FIELD OF THE INVENTION

The present invention relates to compositions, methods and kits for treatment and diagnosis of patients with cancer, and more specifically to inhibiting or reducing the recurrence or metastasis of cancer and preventing the development of therapeutic resistance.

### BACKGROUND OF THE INVENTION

Cancers of epithelial origin account for 90% of cancer deaths worldwide.

The standard treatment of most potentially curable solid tumors is surgical removal often followed by chemotherapy. For the major cancer killers such as lung, breast, and colorectal cancer, the administration of chemotherapy after the tumour is surgically removed may eradicate micrometastatic disease (disease undetectable using conventional imaging technologies) in those patients who still harbor residual cancer cells after surgery. However, this treatment is often unsuccessful.

The resistance of any given cancer cell to conventional medical treatments may not primarily result from the possession or acquisition of specific point mutations but instead largely reside in a distinct cancer cell subpopulation of cancer stem cells. In addition to being relatively resistant to conventional medical therapies, cancer stem cells are also capable of metastasis and tissue colonization. As few as 200 cancer cells displaying the stem cell phenotype can form tumours in animal models, while 20,000 cancer cells without the stem cell phenotype fail to form tumours. These cells are therefore particularly relevant to cancer metastasis and recurrence and treatment resistance.

Thus, there is a need for new therapeutic strategies in treating patients with cancer.

### SUMMARY OF THE INVENTION

In one aspect, there is provided a method for preventing or inhibiting metastasis and/or recurrence of a cancer and/or drug resistance in a patient after a primary treatment of the patient, the method comprising: (a) administering a therapeutically effective amount of a compound or composition for inhibiting stem cell enrichment in any surviving cancer cell population. In one embodiment, inhibiting stem cell enrichment comprises inhibiting at least one of: cancer stem cell self-renewal and induction of epithelial-mesenchymal transition in a cancer cell.

In one embodiment, the composition is administered perioperatively. The compound or composition may be administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

In one embodiment, the primary treatment is one of endocrine therapy, chemotherapy, radiotherapy, hormone therapy, surgery, gene therapy, thermal therapy, and ultrasound therapy. In one embodiment, the primary treatment is excision of a solid tumor.

The compound or composition may comprises a non-steroidal anti-inflammatory drug, a heparin, cytotoxic chemotherapy or a cytokine inhibitor.

In one embodiment, the compound or composition comprises a cytokine inhibitor. In one embodiment, the compound or composition comprises one or more antibodies specific for at least one cytokine involved in stem cell enrichment. The cytokine suitably comprises at least one of: TGF-beta, HGF, IL-6, PGE-2, MCP-1, MMP-9, PDGF-BB and PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

In various embodiments, the compound or composition inhibits the upregulation of the one or more cytokines for at least about 48 hours, at least about 72 hours, at least about 96 hours, at least about 120 hours or at least about 1 week post primary treatment.

In one embodiment, the compound or composition comprises a neu-1 sialidase inhibitor, preferably oseltamivir phosphate.

In one embodiment, the composition further comprises a therapeutically effective amount of a further therapeutic agent selected from: a non-steroidal anti-inflammatory drug, a heparin and cytotoxic chemotherapy.

In another aspect, there is provided a method for treating cancer in a patient, the method comprising: (a) administering a primary treatment to the patient; and (b) administering a compound or composition for inhibiting stem cell enrichment in any surviving cancer cell population; wherein the compound or composition is administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

In another aspect, there is provided a pharmaceutical composition for preventing or inhibiting metastasis or recurrence of a cancer or drug resistance in a patient after a primary treatment of the cancer, the composition comprising a pharmaceutically acceptable carrier and a therapeutically effective amount of an inhibitor of at least one cytokine associated with stem cell enrichment.

In one embodiment, the inhibitor is an antibody specific for the at least one cytokine associated with stem cell enrichment. The cytokine(s) may be selected from: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

In one embodiment, the composition includes a further therapeutic agent selected from: a non-steroidal anti-inflammatory drug, a heparin and cytotoxic chemotherapy.

In one embodiment, the composition is administered perioperatively. The composition may be administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

In another aspect, there is provided a prophylactic method of inhibiting a risk of metastasis or recurrence of a cancer or drug resistance in a patient diagnosed with the cancer, the method comprising: (a) administering a therapeutically effective amount of a composition for inhibiting stem cell enrichment.

In one embodiment, the composition is administered prior to a primary treatment. In one embodiment, perioperatively.

The primary treatment may be one of endocrine therapy, chemotherapy, radiotherapy, hormone therapy, surgery, gene therapy, thermal therapy, and ultrasound therapy.

In one embodiment, the composition comprises an inhibitor of at least one cytokine associated with stem cell enrichment. The cytokine may be at least one of: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

In one embodiment, the composition includes a therapeutically effective amount of a therapeutic agent selected from: a non-steroidal anti-inflammatory drug, a heparin and cytotoxic chemotherapy.

In another aspect, there is provided a method of determining whether a patient is at risk of metastasis or recurrence of a cancer after primary treatment of the cancer, the method comprising: determining a level of at least one cytokine associated with stem cell enrichment in a sample of the patient after the primary treatment; wherein a higher level of the at least one cytokine correlates to a higher risk of recurrence. The cytokine(s) may be selected from: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

The level may be determined at least one of 30, 29, 28, 27, 26, 25, 24, 23, 22, 21, 20, 19, 18, 17, 16, 15, 14, 13, 12, 11, 10, 9, 8, 7, 6, 5, 4, 3, 2 and 1 day(s) after the primary treatment, and 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 and 0.1 hour(s) after the primary treatment, and immediately after the primary treatment, and prior to the primary treatment. In one embodiment, the level is determined in a patient sample, and the sample is selected from the group consisting of: blood sample, serum sample, tissue sample and tumour sample. The level may be determined by mRNA or protein level analysis.

In another aspect, there is provided a kit for use in a method described herein, the kit comprising: one or more antibodies specific for a cytokine associated with stem cell enrichment.

In one aspect, there is provided a method of determining a risk of metastasis or recurrence associated with a cancer in a patient after a primary therapy, the method comprising: (a) determining activity levels associated with at least one cytokine involved in stem cell enrichment in a sample of the patient after the primary therapy; (b) constructing an activity profile of the patient from the activity levels; (c) comparing the activity profile to a reference activity profile with a predetermined risk of metastasis or recurrence; and wherein if the activity profile has a value greater than that of the reference activity profile, then the risk of metastasis or recurrence is greater than the reference activity profile, and if the activity profile has a value less than that of the reference activity profile, then the risk of metastasis or recurrence is lower than the reference activity profile. In one embodiment, the activity levels are determined by mRNA level or protein level analysis. At least one cytokine may be selected from the group consisting of: TGF-beta, HGF, IL-6, PGE-2, PGF, PDGF-BB, MCP-1 and MMP-9; in one embodiment, the method includes determining the activity levels of HGF and IL-6, and optionally one or more of TGF-beta, PGE-2, MCP-1, MMP-9, PDGF-BB and PGF; and more preferably all of HGF, IL-6, TGF-beta, PGE-2, PDGF-BB.

In one embodiment, the reference activity profile is that of a patient who does not have metastasis or recurrence of the cancer for a predetermined period of time after primary surgery. In one embodiment, the reference activity profile is that of the patient prior to primary treatment.

The activity levels may be determined at one or more of 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment. In one embodiment, the sample is one of a blood sample, tumour sample, serum sample and tissue sample of the patient.

In another aspect, there is provided a method for preventing or inhibiting metastasis or recurrence of a cancer or drug resistance in a patient after a primary treatment of the patient, the method comprising: (a) interfering with the cellular repair mechanisms invoked by the cancer cells after the primary treatment.

In another aspect, there is provided a method of preventing or inhibiting metastasis or recurrence of a cancer or drug resistance in a patient after a primary treatment of the patient comprising: (a) administering a therapeutically effective amount of a cancer therapy targeted towards a population of proliferating cancer stem cells in the patient.

In another aspect, there is provided a method of treating a cancer in a patient comprising: (a) administering a treatment that induces a population of cancer stems cells in the patient to proliferate; and (b) administering a therapeutically effective amount of a cancer therapy targeted towards the proliferating cancer stem cells in the patient.

In another aspect, there is provided a method of inhibiting metastasis or recurrence of a cancer in a patient after a primary treatment of the patient comprising: (a) obtaining a patient sample; (b) determining a cancer stem cell proliferation profile for the patient; (c) comparing the cancer stem cell proliferation profile to a reference cancer stem cell proliferation profile; and (d) administering a therapeutically effective amount of a cancer therapy targeted towards a population of proliferating cancer stem cells in the patient if the cancer stem cell proliferation profile is greater than or equal to the reference cancer stem cell proliferation profile. In one embodiment, determining the cancer stem cell proliferation profile comprises determining a concentration of markers in the patient sample indicative of cancer stem cell proliferation. In one embodiment, determining the cancer stem cell proliferation profile comprises determining the rate of proliferation of cancer stem cells in the patient sample. The patient sample may be obtained at one or more of the group consisting of: prior to primary treatment, 1 hour after primary treatment, within 6 hours after primary treatment, within 12 hours after primary treatment, within 18 hours after primary treatment, within 24 hours after primary treatment, within 30 hours after primary treatment, within 36 hours after primary treatment, within 42 hours after primary treatment, within 48 hours after primary treatment, within 54 hours after primary treatment, within 60 hours after primary treatment, within 66 hours after primary treatment, within 72 hours after primary treatment, within 78 hours after primary treatment, within 84 hours after primary treatment, within 90 hours after primary treatment and within 96 hours after primary treatment.

In one embodiment, the cancer therapy is administered when the cancer stem cell proliferation profile is greater than or equal to the reference cancer stem cell proliferation profile.

In various embodiments, the cancer therapy is administered within; 6 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 120 hours or within 1 week after administering a treatment that induces a population of cancer stems cells in the patient to proliferate or after primary treatment.

In various embodiments, the cancer therapy may be one or more of the group consisting of endocrine therapy, chemotherapy, hormone therapy, gene therapy, thermal therapy, ultrasound therapy and immunotherapy. In one embodiment, nanoparticle-mediated thermal therapy. In one embodiment, cytotoxic chemotherapy, which in various embodiments, may be selected from alkylators (including cyclophosphamide/cisplatin/melphalan); topoisomerase inhibitors 1 and 2 (including doxorubicin/irinotecan/etoposide/topotecan); taxanes (including docetaxel/paclitaxel/abraxane); vinca alkaloids (including vincristine/vinblastine); and antimetabolites (including 5-FU/Gemcitabine/Cytarabine/Pemetrexed).

The method described above wherein administering the cancer therapy targeted towards the population of proliferating cancer stem cells may inhibit proliferation of the population of cancer stem cells or induce apoptosis in the population of cancer stem cells.

In another aspect, there is provided a method of treating cancer in a patient comprising: perioperatively administering an inhibitor of one or more cytokines selected from: TGF-beta, IL-6, MCP-1, PGE-2, PDGF-BB, MMP-9 and PGF, wherein an inhibitor of HGF is not administered; and perioperatively administering cytotoxic chemotherapy targeted to proliferating cancer cells. The cytotoxic chemotherapy may be selected from alkylators (including cyclophosphamide/cisplatin/melphalan); topoisomerase inhibitors 1 and 2 (including doxorubicin/irinotecan/etoposide/topotecan); taxanes (including docetaxel/paclitaxel/abraxane); vinca alkaloids (including vincristine/vinblastine); and antimetabolites (including 5-FU/Gemcitabine/Cytarabine/Pemetrexed). In various embodiments, the cytotoxic chemotherapy and/or cytokine inhibitor are administered within: 6 hours, 12 hours, 18 hours, 24 hours, 30 hours, 36 hours, 42 hours, 48 hours, 54 hours, 60 hours, 66 hours, 72 hours, 78 hours, 84 hours, 90 hours, 96 hours, 120 hours or within 1 week after surgery to remove a solid tumour.

### BRIEF DESCRIPTION OF THE DRAWINGS

FIG. 1 depicts the expression of cytokines after tumour removal. Tumour removal is shown to lead to a drop in TGF-beta and PDGFBB levels, which triggers increased stromal secretion of HGF, PGE-2 and IL-6. TGF-beta and PDGFBB levels will rapidly rise to baseline and in concert with HGF, IL-6, and PGE-2 facilitate cellular proliferation and stem cell enrichment in a residual cancer cell population.

FIG. 2 depicts scatterplots of flow cytometry experiments from enriched circulating tumour cells stained with an antibody containing CD44 following treatment with various cytokines and cytokine cocktails.

FIG. 3 depicts scatterplots of low cytometry from enriched circulating tumour cells stained with an antibody containing CD133 following treatment with various cytokines and cytokine cocktails.

FIG. 4 depicts cell proliferation of various cell subpopulations in the HCT-15 cell line after exposure to various cytokines and cytokine cocktails.

FIG. 5 depicts cell proliferation of various cell subpopulations in the SW 620 cell line after exposure to various cytokines and cytokine cocktails.

FIG. 6 depicts cell proliferation of various cell subpopulations in cultured circulating tumour cells after exposure to various cytokines and cytokine cocktails. Cells were stained using an antibody containing CD44 PE.

FIG. 7 depicts cell proliferation of various cell subpopulations in cultured circulating tumour cells after exposure to various cytokines and cytokine cocktails. Cells were stained using an antibody containing CD133 PE.

FIG. 8 depicts enriched circulating tumour cells cultured with or without cytokines, including IL-6, IL-S and PDGF-BB. The addition of IL-6 significantly increased (p<0.05) the subpopulation of EpCAM+CD133− cells as compared to control.

FIG. 9 depicts flow cytometry scatterplots of enriched circulating tumour cells stained with EpCAM A488, CD133PE and Lgr5-PE-Vio770 following treatment with IL-6, IL-8 and PDGFBB.

FIG. 10 depicts the percentage of CD44+CD133− cells following treatment with various cytokine and cytokine cocktails.

FIG. 11 depicts the percentage of CD44+CD133− cells following treatment with various cytokines and Irinotecan.

FIG. 12 depicts the percentage of CD44+CD133− cells following treatment with various cytokine cocktails and Irinotecan.

FIG. 13 depicts the effect of treatment of various cytokines on cellular apoptosis.

FIG. 14 depicts the effect of treatment of various cytokines and Irinotecan on cellular apoptosis.

FIG. 15 depicts the effect of treatment of various cytokine cocktails and Irinotecan on cellular apoptosis.

FIG. 16 depicts the percentage of CD44−CD133+ cells following treatment with various cytokines and cytokines cocktails.

FIG. 17 depicts the percentage of CD44−CD133+ cells following treatment with various cytokines and Irinotecan.

FIG. 18 depicts the percentage of CD44−CD133+ cells following treatment with various cytokine cocktails and Irinotecan.

FIG. 19 depicts the percentage of CD44+CD133+ cells following treatment with various cytokine and cytokine cocktails.

FIG. 20 depicts the percentage of CD44+CD133+ cells following treatment with various cytokines and Irinotecan.

FIG. 21 depicts the percentage of CD44+CD133+ cells following treatment with various cytokine cocktails and Irinotecan.

### DETAILED DESCRIPTION

In the following description, numerous specific details are set forth to provide a thorough understanding of the invention. However, it is understood that the invention may be practiced without these specific details.

Cancer Stem Cells and Metastatic Cancer

In contrast to the proven ability of chemotherapy to cure micrometastatic cancer in some patients, clinically evident metastatic cancer is generally incurable. Given the emerging evidence of the importance of cancer stem cells in drug resistance and metastatic efficiency, the eradication of this cancer cell subpopulation may be critical to achieve cancer cure.

Differentiated cancer cells may dedifferentiate to a cancer stein cell phenotype, either spontaneously, or, after certain triggering mechanisms. After an initial treatment against cancer, such as surgery, chemotherapy, or radiation, tissue damage induced by that treatment will trigger the release of specific inflammatory molecules fostering the induction of a partial epithelial-mesenchymal transition (EMT) in the remnant cancer cell population and reversion to a cancer stem cell phenotype. The present inventor provides evidence that these same signaling pathways foster cancer stem cell self-renewal as a highly conserved response to tissue damage. The net result of this process is the rapid emergence of a stem cell enriched residual cancer cell population. By targeting this highly conserved signaling pathway at the time it is upregulated, a novel and highly effective medical treatment against cancer results.

EMT induction confers a migratory and invasive epithelial phenotype critical to tissue repair; stem cell activation provides the epithelial cell precursors necessary to regenerate tissue. A rapid, even transient, phenotypic shift in cancer cells to a more migratory, metastatic phenotype in response to a treatment induced release of tissue repair signals may have significant clinical implications. Cancer cell populations that are enriched for cancer stem cells are highly resistant to ionizing radiation, conventional chemotherapy, and highly tumorigenic. Disrupting the highly conserved cytokine/signaling pathways important in EMT induction and stem cell activation at the time they are released provides a treatment strategy against residual cancer cells, preventing the emergence of a treatment resistant, metastatic cancer cell phenotype. Although this clinical strategy could be employed after any treatment damaging the tumor, it can be particularly effective in the potentially curative adjuvant setting after the primary tumor has been surgically removed, limiting the ability of any surviving cancer cell population to proliferate and metastasize.

The present inventor further provides evidence that by administering treatments that interfere with the repair (regrowth) of the tumor after any treatment that will damage the tumor (surgery, chemotherapy, radiation therapy) the development of treatment resistance will be significantly mitigated primarily through preventing enrichment for treatment resistant cancer stem cells.

In one aspect, there is provided a method for inhibiting metastasis or recurrence of a cancer and the development of therapeutic resistance in a patient after a primary treatment of the patient, the method comprising administering a therapeutically effective amount of a compound or composition for inhibiting stem cell enrichment in any surviving cancer cell population.

The term “cancer”, as used herein, may mean a malignant neoplasm that has undergone characteristic anaplasia with loss of differentiation, increased rate of growth, invasion of surrounding tissue, and is capable of metastasis. Residual cancer is cancer that remains in a subject after any form of treatment given to the subject to reduce or eradicate a cancer and recurrent cancer is cancer that recurs after such treatment. Metastatic cancer is a cancer at one or more sites in the body other than the site of origin of the original (primary) cancer from which the metastatic cancer is derived. In the case of a metastatic cancer originating from a solid tumor, one or more (for example, many) additional tumor masses can be present at sites near or distant to the site of the original tumor. The phrase “disseminated metastatic nodules” or “disseminated metastatic tumors” refers to a plurality (typically many) metastatic tumors dispersed to one or more anatomical sites. For example, disseminated metastatic nodules within the peritoneum (that is a disseminated intraperitoneal cancer) can arise from a tumor of an organ residing within or outside the peritoneum, and can be localized to numerous sites within the peritoneum. Such metastatic tumors can themselves be discretely localized to the surface of an organ, or can invade the underlying tissue.

In an aspect, the cancer originates from a solid tumour.

The term “tumor”, as used herein, refers to a neoplasm or an abnormal mass of tissue that is not inflammatory, which arises from cells of pre-existent tissue. A tumor can be either benign (noncancerous) or malignant (cancerous). Tumors can be solid or hematological. Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelogenous leukemia, and chronic lymphocytic leukemia), myelodysplastic syndrome, and myelodysplasia, polycythemia vera, lymphoma, (such as Hodgkin's disease, all forms of non-Hodgkin's lymphoma), multiple myeloma, Waldenstrom's macroglobulinernia, and heavy chain disease. Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, lung cancer, ovarian cancer, prostate cancer, benign prostatic hyperplasia, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, epithelial tumors (e.g., cervical cancer, gastric cancer, skin cancer, head and neck tumors), testicular tumor, bladder carcinoma, melanoma, brain tumors, and CNS tumors (such as a glioma, astrocytoma, medulloblastoma, craniopharyogioma, ependymoma, pinealoma, hemangioblastoma, acoustic neuroma, oligodendroglioma, menangioma, meningioma, neuroblastoma and retinoblastoma).

In some aspects, the tumour is a malignant solid tumour.

As used herein, the term “metastasis” refers to the growth of a cancerous tumor in an organ or body part, which is not directly connected to the organ of the original cancerous tumor. Metastasis may be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, or primary tumour, and migration and/or invasion of cancer cells to other parts of the body.

In some aspects, metastasis refers to the subsequent growth or appearance of a cancerous tumour in a different location to an original tumour after treatment of the original tumour.

As used herein, the terms “recurrence” and grammatical variants thereof, refer to further growth of neoplastic or cancerous cells after diagnosis of cancer or a primary tumour. Particularly, recurrence may occur when further cancerous cell growth occurs in the cancerous tissue at the site of the original cancer. The cancer may come back to the same place as the original cancer/primary tumor or to another place in the body.

In some aspects, recurrence refers to a cancer that has reappeared at the site of an original cancer or primary tumour after treatment of that original cancer or primary tumour, after a period of time during which the cancer or tumour could not be detected.

The term “treatment” as used herein generally means obtaining a desired physiologic effect. The effect may be prophylactic in terms of completely or partially preventing a disease or condition or symptom thereof and/or may be therapeutic in terms of a partial or complete cure for an injury, disease or condition and/or amelioration of an adverse effect attributable to the injury, disease or condition and includes arresting the development or causing regression of a disease or condition.

“Primary treatment”, as used herein, means any treatment of any kind or means intended to or having the effect of partially or completely removing, destroying, damaging, excising, reducing in size, rendering benign or inhibiting the growth of, a cancer or tumour, and may include one or more such treatments. For example, primary treatment may include one or more of endocrine therapy, chemotherapy, radiotherapy, hormone therapy, surgery, gene therapy, thermal therapy, and ultrasound therapy.

In an aspect, the primary treatment is surgical excision of a solid tumour.

“Cancer stem cells”, as used herein, are defined and functionally characterized as a small subset of cells from a tumor that can grow indefinitely in vitro under appropriate conditions (i.e., possess the ability for self-renewal), and are able to form tumors in vivo using only a small number of cells. Other common approaches to characterize cancer stem cells involve morphology and examination of cell surface markers, transcriptional profile, and drug response.

“Stem cell enrichment”, as used herein, means the increase in size or proportion or concentration of a population of cancer stem cells locally at the site of a cancer or tumour in a patient or in a location distal to the cancer or tumour. Stem cell enrichment may, in some aspects, include cancer stem-cell self-renewal, partial or complete induction of epithelial-mesenchymal transition in a cancer cell, or cancer stem cell proliferation.

### Methods, Compounds and Compositions

In one aspect, there is provided methods, compounds and compositions for inhibiting metastasis, treatment resistance or recurrence of a cancer in a patient after a primary treatment of the patient. In one embodiment, the method comprises administering a therapeutically effective amount of a compound or composition for inhibiting stem cell enrichment in any surviving cancer cell population.

As used herein in one embodiment, a cancer patient refers to a mammal with cancer, in one embodiment, a human patient diagnosed with cancer.

In one aspect the compound or composition is administered perioperatively. In one embodiment, “perioperatively” or “in the perioperative period”, include the preoperative, intraoperative and postoperative period that terminates with the resolution of the surgical sequelae. In various embodiments, the composition is administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects. In one embodiment, “therapeutically effective amount”, as used herein, means a level that inhibits or prevents the upregulation or the activity of one of more cytokines associated with stem cell enrichment.

As used herein, “therapeutic agent” means any chemical or biological material, and may be a compound or composition, suitable for administration by methods known to those in the art, which induces a desired biological or pharmacological effect. The effect may be local or it may be systemic.

In one embodiment, compounds or compositions as described herein suitably comprise an inhibitor of one or more cytokines associated with stem cell enrichment. “Inhibitor” includes, but is not necessarily limited to, an antibody, a soluble cytokine binding protein (e.g. a soluble cytokine receptor) and a receptor antagonist.

In other embodiments, the compounds or compositions may comprise inhibitors of downstream molecules activated by the receptor-ligand interaction of the identified cytokines.

In one aspect, a therapeutically effective amount of a compound or composition that inhibits the upregulation of one or more cytokines associated with stem cell enrichment is administered before surgery and/or after surgery, wherein in various embodiments, the compound or composition inhibits the upregulation of the one or more cytokines for at least about 48 hours, at least about 72 hours, at least about 96 hours, or at least about 120 hours post primary treatment.

In various embodiments, the duration of treatment may be up until 96 hours post-primary treatment, up to about 120 hours post primary treatment or up to about 144 hours post primary treatment.

In one aspect the compound or composition is administered before surgery and/or after surgery to maintain an effective level of the inhibitor(s) for at least 96 to 120 hours post primary treatment. In one embodiment, administration of the composition ceases such that circulating levels of the inhibitor(s) decline after 96 to 120 hours post primary treatment.

In yet other aspects, the compound or composition is administered prior to 1 week after the primary treatment.

In yet other aspects, the compound or composition is administered prior to 5 days after the primary treatment.

In yet other aspects, the compound or composition is administered prior to 4 days after the primary treatment.

In still other aspects, the compound or composition is administered prior to 3 days after the primary treatment.

In still other aspects, the compound or composition is administered within 48 hours after the primary treatment.

In still another aspect, the compound or composition is administered within 24 hours +/−12 hours after primary treatment.

In still another aspect, the compound or composition is administered immediately after or simultaneous with the primary treatment.

In one embodiment, the composition comprises one or more antibodies specific for at least one cytokine involved in stem cell enrichment.

After primary treatment, distinct cytokines, including, but not necessarily limited to, TGF-beta, Hepatocyte Growth Factor (HGF), Interleukin 6 (IL-6), prostaglandin E2 (PGE-2), Matrix metallopeptidase 9 (MMP-9), Monocyte Chemoattractant Protein 1 (MCP-1), Platelet derived growth factor BB (PDGF-BB) and placental growth factor (PGF), may be released at a predictable time frame after treatment, particularly after any treatment that damages a cancerous tumor. These cytokines may facilitate self-renewal of normally dormant cancer stem cells, and/or may facilitate the dedifferentiation of more differentiated cancer cells to a stem cell phenotype via induction of a partial EMT. Targeting this cytokine signaling network induced by cancer treatments at the time these signaling networks are upregulated can limit the ability of a residual cancer cell population to repair itself after any treatment that has damaged it, including radiation therapy, cytotoxic chemotherapy, and surgery. The same signaling pathways that trigger cancer stem cell proliferation may also facilitate stem cell enrichment by facilitating the molecular reprogramming of more differentiated cancer cells via a partial EMT and transition to the stem cell phenotype. By targeting this highly conserved signaling pathway at the time it is upregulated a novel medical treatment against cancer is provided.

In one embodiment, the targeting of these molecules is not particularly restricted, and may be performed using multiple different technologies, including monoclonal antibodies against one or more of the cytokines implicated in triggering cancer stem cell self-renewal and EMT pathways, including one or more of the upregulated cytokines identified above.

TGF-beta is secreted by the cancer cell as a latent complex stored in the ECM. Myofibroblasts release bioactive TOE-beta from the latent complex through proteolytic and non-proteolytic mechanisms. Without wishing to be bound by a theory, the present inventor has observed that TGF-beta decreases at 24 hours after primary treatment, and then rapidly increases to normal or above baseline levels; it is postulated that this sudden drop causes the remaining cancer cell population to be more sensitive to the effects of acute inflammatory mediators, including IL-6, HGF, PGE-2, PGF, PDGFBB, MCP-1 and MCP-9, and other known inflammatory mediators. Given the pleiotropic nature of TGF-beta, its sudden drop after initial cancer treatments followed by rapid increase may serve as an initial molecular trigger that facilitates the transition to a stem-cell enriched residual cancer cell population. It has been observed that PDGF-BB decreases and then increases over the same time period in a similar manner to TGF-beta and therefore may also be a part of this molecular trigger. Inventor has documented that both TGF-beta and PDGF-BB, after initial drop, will rapidly return to baseline or above. This fluctuation is predicted to sensitize a residual cancer cell population to the effects of an increase in cytokines such as 11-6 and HGF among others. In concert with these upregulated cytokines and the return to normal or higher levels of PDGF-BB and TGF-beta, an environment is created conducive to stem cell proliferation, EMT, and residual cancer cell proliferation.

In various embodiments, the compound or composition inhibits at least one, at least two, at least three, at least four at least five, at least six, at least seven and preferably all of HGF, IL-6, PGE-2, MCP-1, MMP-9, TGF-beta, PDGF-BB, and PGF. In one embodiment, the compound or composition inhibits at least HGF and IL-6.

In yet other aspects, there is provided compositions comprising therapeutically effective amounts of one or more antibodies specific for at least one of the group consisting of: TGF-beta, HGF, IL-6, PGE-2, MCP-1, MMP-9, PDGF-BB, and PGF. In one embodiment, one or more of an antibody specific for HGF, IL-6, PGE-2, MCP-1, MMP-9 and PGF. In one embodiment, the composition includes an antibody specific for IL-6 and HGF. In one embodiment, the compound or composition inhibits HGF, IL-6, TGF-beta, PGE-2, and PDGF-BB, and PGE

The terms “antibody”, “antibodies” and “immunoglobulin”, as used herein, refer broadly to any immunological binding agent or molecule that comprises a human antigen binding domain, including polyclonal and monoclonal antibodies. Depending on the type of constant domain in the heavy chains, whole antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known. As will be understood by those in the art, the immunological binding reagents encompassed by the term “antibody” extend to all human antibodies and antigen binding fragments thereof; including whole antibodies, dimeric, trimeric and multimeric antibodies; bispecific antibodies; chimeric antibodies; recombinant and engineered antibodies, and fragments thereof. The term “antibody” is thus used to refer to any human antibody-like molecule that has an antigen binding region, and this term includes antibody fragments that comprise an antigen binding domain such as Fab′, Fab, F(ab′)₂, single domain antibodies (DABs), T and Abs dimer, Fv, scFv (single chain Fv), dsPv, ds-scFv, Fd, linear antibodies, minibodies, diabodies, bispecific antibody fragments and the like.

Methods for preparing antibodies to IL-6 are known and are disclosed, for example in U.S. Pat. No. 5,670,373, U.S. Pat. No. 5,866,689, CA2,700,498, CA2,763,039 CA2,632628, U.S. Pat. No. 6,235,28, U.S. Pat. No. 5,959,085, U.S. Pat. No. 7,482,436 incorporated herein by reference.

Methods for preparing antibodies to HGF are known and are disclosed, for example in U.S. Pat. No. 7,718,174, CA2,472,383, CA2,524329, U.S. Pat. No. 6,099,841, incorporated herein by reference.

Methods for preparing antibodies to PGE-2 are known and are disclosed, for example in CA2,812,756, CA2,664,763, U.S. Pat. No. 8,624,002, incorporated herein by reference.

Methods for preparing antibodies to PGF are known and are disclosed, for example in CA2,607,471, U.S. Pat. No. 7,482,004, CA2,601,267, incorporated herein by reference.

Methods for preparing antibodies to MCP-1 are known and are disclosed, for example in CA2,609,349, U.S. Pat. No. 7,342,106 EP1,888,114, U.S. Pat. No. 7,687,606, incorporated herein by reference.

Methods for preparing antibodies to MMP-9 are known and are disclosed, for example in U.S. Pat. No. 8,013,125, U.S. Pat. No. 9,120,863, U.S. Pat. Nos. 8,999,332, 2,379,373, U.S. Pat. No. 8,008,445, incorporated herein by reference.

The antibody may be modified by attachment with various molecules such as an enzyme, a fluorescent material, a radioactive material and a protein. The modified antibody may be obtained by chemically modifying the antibody. This modification method is conventionally used in the art. Also, the antibody may be obtained as a chimeric antibody having a variable region derived from a non-human antibody, and a constant region derived from a human antibody, or may be obtained as a humanized antibody including a complementarity-determining region derived from a non-human antibody, and a framework region (FR) and a constant region derived from a human antibody. Such an antibody may be prepared by using a method known in the art.

In some embodiments, the inhibitor(s) of one or more cytokines associated with stem cell enrichment as described above are used in combination with a further therapeutic agent.

In some embodiments, the compound or composition includes antibodies against one or more of IL-6, HGF, PGE-2, PGF, TGF-beta, PDGF-BB, MCP-1 and MMP-9 or their receptors. In another embodiment, the compound or composition is or includes a neu-1 sialidase inhibitor such as oseltamivir phosphate that can prevent receptor dimerization triggered by these ligand-receptor interactions and hence prevent downstream activation. In other embodiments, the compound or composition is or includes a small molecule inhibitor of the transcriptional activators triggered by these distinct ligand-receptor interactions, including inhibitors of transcriptional activators such as NF-kb and Stat-3, among others. Other therapeutic agents that may be employed to disrupt the effects of these distinct ligand receptor interactions are miRNA therapeutics that disrupt the post-transcriptional activity of target genes upregulated by the distinct ligand-receptor intereractions described above.

In some embodiments, a composition is provided comprising at least one inhibitor of one or more cytokines associated with stem cell enrichment and a therapeutically effective amount of a further therapeutic agent.

In one aspect, the therapeutic agent is an anti-cancer drug. In other aspects, the therapeutic agent may be at least one of: a non-steroidal anti-inflammatory drug, a heparin and cytotoxic chemotherapy.

In another aspect, there is provided a pharmaceutical composition for inhibiting metastasis, recurrence of a cancer and/or therapeutic resistance in a patient after a primary treatment of the cancer, the composition comprising a pharmaceutically acceptable carrier and a therapeutically effective amount of at least one antibody specific for at least one cytokine involved in stem cell enrichment.

The compositions of the present disclosure can be administered in any manner which enables inhibition of the effects of the molecules inducing cancer stem cell self-renewal and EMT pathways in cancer cells. The composition may be injected in a pharmaceutically acceptable liquid carrier directly to the site of injury. Alternatively, the composition may be administered intravenously or orally. Depending on the isolation of the primary tumour, other modes of administration may be appropriate.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. Pharmaceutically acceptable carriers must be compatible with both the components of the composition and the patient. Other examples of non-aqueous solvents include propylene glycol and other glycols, metabolizable oils, aqueous carriers including water and alcoholic/aqueous solutions, and emulsions or suspensions (eg. saline and buffered media).

Suitable dosage ranges may be readily ascertained by those of skill in the art.

Monitoring of Cytokine Levels Post Primary Treatment

Acute inflammatory mediators and cytokine levels important in physiologic wound repair, EMT induction, and stem cell activation may be evaluated immediately before and after initial treatment against cancer. Post-treatment sampling can suitably be taken at 24, 48, 72, and 96 hours after treatment as well as one, two weeks, and one month after treatment. These time points encompass the time frame associated with the acute and proliferative phases of a physiologic wound repair response. In some embodiments, samples may be taken at one or more of 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 02 or 0.1 hour(s) after the primary treatment, or immediately after the primary treatment, or prior to the primary treatment

In some embodiments, there is provided a method of determining whether a patient is at risk of metastasis or recurrence of a cancer after primary treatment of the cancer, the method comprising determining a level of at least one cytokine associated with stem cell enrichment in a sample of the patient after the primary treatment, wherein a higher level of at least one cytokine correlates to a higher risk of recurrence.

In one embodiment, the at least one cytokine is one or more of HGF, IL-6, PGE-2, MMP-9, PDGF-BB, TGF-beta, MCP-1, and PGF.

In some aspects, the level is determined at one or more of 30, 29, 28, 27, 26, 25, 24, 23, 22, 21, 20, 19, 18, 17, 16, 15, 14, 13, 12, 11, 10, 9, 8, 7, 6, 5, 4, 3, 2 and day(s) after the primary treatment, and 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 and 0.1 hour(s) after the primary treatment, and immediately after the primary treatment, and prior to the primary treatment.

In one embodiment, the level is determined at a time point between about 24 hours and about 96 hours post-primary treatment. In another embodiment, the level is obtained one week after surgical removal of a primary tumor. Persistent upregulation of TGF-beta and PDGFBB above baseline predicts residual micrometastatic disease and hence benefit of adjuvant systemic therapies.

In an aspect, the level is determined in a patient sample selected from the group consisting of: blood sample, serum sample, tissue sample and tumour sample. In an aspect, the level is determined by mRNA or protein analysis.

In a further aspect, there is provided a prophylactic method of inhibiting a risk of metastasis, recurrence of a cancer and/or therapeutic resistance in a patient diagnosed with the cancer, the method comprising, administering a therapeutically effective amount of a compound or composition for inhibiting stem cell enrichment.

In some embodiments, a “wound healing pattern” may be detectable in the serum or plasma of patients after primary treatment. This pattern may reveal upregulated expression of growth factors/cytokines with established roles in EMT induction such as TGF-beta, HGF, IL-6, PGE-2, PGF, PDGF-BB, MCP-1 and MMP-9 as well as other molecules important in stem cell activation or cancer stem cell activation. In some embodiments, the method may comprise taking samples for molecular expression profiles during the perioperative period in both benign and neoplastic primary treatments to assist in constructing reference expression profiles. For example, in some embodiments, the comparison may be in respect of acute inflammatory expression profile in patients who undergo wide local excision of a breast carcinoma with patients who have surgical removal of benign tumors such as fibroadenomas. Patient outcomes may also be determined to assist in associating the expression profiles and reference expression profiles with patient prognosis.

In another aspect, there is provided a method of determining a risk of metastasis or recurrence associated with a cancer in a patient after a primary therapy, the method comprising, determining activity levels associated with at least one cytokine involved in stem cell enrichment in a sample of the patient after the primary therapy; constructing an activity profile of the patient from the activity levels; comparing the activity profile to a reference activity profile with a predetermined risk of metastasis or recurrence; and wherein if the activity profile has a value greater than that of the reference activity profile, then the risk of metastasis or recurrence is greater than the reference activity profile, and if the activity profile has a value less than that of the reference activity profile, then the risk of metastasis or recurrence is lower than the reference activity profile.

In a further aspect, there is provided a method for inhibiting metastasis or recurrence of a cancer in a patient after a primary treatment of the patient, the method comprising, interfering with the cellular repair mechanisms invoiced by the cancer cells after the primary treatment.

In some aspects, the activity levels are determined by mRNA level or protein level analysis. In some aspects, at least one cytokine is selected from the group consisting of: TGF-beta, HGF, IL-6, PGE-2, MMP-9, PDGF-BB, MCP-1 and PGF. In other aspects, the reference activity profile is that of a patient who does not have metastasis or recurrence of the cancer as measured at a predetermined period of time after primary surgery. In some aspects, the activity levels are determined at one or more of 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment. The sample may be one of a blood sample, tumour sample, serum sample and tissue sample of the patient.

In some embodiments, there is provided a method for inhibiting metastasis or recurrence of a cancer in a patient after a primary treatment of the patient, the method comprising interfering with the cellular repair mechanisms invoked by the cancer cells after the primary treatment.

### Primary Tumour Removal Activates Wound Healing Response and Enhances Proliferation and Survival of Residual Cancer Cells

### Normal Tissues

The first phase of wound healing occurs immediately after tissue injury and lasts for approximately 3 days. This is the period of hemostasis and inflammation. Tissue injury leads to platelet activation and the triggering of the coagulation cascade, resulting in the release/formation of various inflammatory mediators such as platelet activating factor, thrombin, and histamine. Inflammatory mediators trigger arteriolar vasodilation and an increase in microvascular permeability and they activate the expression of endothelial and inflammatory cell surface adhesive molecules such as the selectins and the integrins, which enhance the ability of circulating inflammatory cells and platelets to overcome circulatory shear forces and bind to the microvasculature.

Bone marrow derived haematopoetic and mesenchymal progenitor cells migrate to sites of injury in response to tissue damage, as do a host of circulating inflammatory cells such as neutrophils and monocytes. Both bone marrow derived progenitor cells and inflammatory cells release reactive oxygen intermediates and proteases critical to matrix degradation and tissue invasion by these cells, and bone marrow derived progenitor cells can synthesize the collagens critical to early wound matrix formation. In response to tissue injury inflammatory cells such as neutrophils and macrophages secrete a wide variety of cytokines such as IL-1 and TNF-alpha, whose pleiotropic effects include endothelial activation and a procoagulant state marked by the increased expression of endothelial adhesion molecules, the upregulation of leukocyte cytokine expression, and the proliferation of fibroblasts.

The growth factors released during this initial phase of inflammation trigger a cascade of additional growth factor release, initiating the second or proliferative phase starting 3 days after wounding and lasting for approximately 10 days. This phase is characterized by the further proliferation of migratory epithelial cells, endothelial cells, and fibroblasts, and the continued synthesis of a collagen rich matrix. The last phase is the remodeling phase, a period of time in which the wound matrix matures and strengthens even further, and this phase occurs from days 8-12 onwards. Note that many of the most important and critical aspects of the wound healing process are completed within one to two weeks after tissue injury.

### Cancerous Cells

The present inventor recognized parallels between hematogenous cancer cell metastasis and the normal wound healing process. For example, tumor derived growth factors are able to choreograph the formation of specific cellular cluster sites or “premetastatic niches” where tumor cells can successfully metastasize to and develop into viable tumors. The complex cellular behaviors involved in forming these pre-metastatic niches parallel in some important respects the events that occur during the normal wound healing process. For example, bone marrow derived progenitor cells, may migrate to sites of tissue injury during the wound healing process, and assist in initiating the premetastatic niche in response to tumor derived factors. Activated fibroblasts proliferate at these niches prior to the arrival of the bone marrow derived progenitors, and similar to their role during wound healing, synthesize the collagens and fibronectins critical to the binding and subsequent proliferation of circulating bone marrow derived progenitors and inflammatory cells. Furthermore, MMP-9 expression is upregulated at these niches in concert with the appearance of the bone marrow derived progenitor cells, an upregulation noted to occur during the normal wound healing response and also facilitated by bone marrow derived progenitor cells.

The present inventor surprisingly discovered that the time course in which some of these complex events occurred after tumor cell implantation is similar to the course of events that occur after tissue injury and during the initial inflammatory and proliferative phases of wound healing. For example, fibronectin expression is increased at sites of future metastasis by day 3 after tumor cell implantation; and resident stromal like fibroblasts proliferate at these niches in concert with the enhanced fibronectin expression by the first week after tumor inoculation.

The inflammatory and coagulation pathways activated by tissue injury may also trigger the release of the growth factors characteristic of the proliferative phase of wound healing. These growth factors can augment the proliferation of residual cancer cells, fibroblasts, and endothelial cells, and some, such as HGF, facilitate the anchorage independent survival of circulating cancer cells and are chemotactic for bone marrow derived progenitor cells. Proliferating fibroblasts trigger the synthesis of the collagens that form the tumor stroma, and proliferating endothelial cells and activated platelets stimulate angiogenesis and the binding of circulating inflammatory cells to the microvasculature through the upregulation of selectin expression.

Accordingly, the present inventor determined that primary tumor treatment can activate wound healing pathways as a result of the tissue damaging trauma, and the seeding of cancer cells into the circulation. Allowing wound healing to occur before initiating therapy can facilitate metastatic spread of the cancer and therapeutic resistance by fostering stem cell enrichment and thereby compromising the effectiveness of subsequent therapy.

Given this reproducible observation seen in a variety of different tumor types, the present inventor determined that growth factor(s) elaborated after surgical removal of the primary tumor triggering residual cancer cell proliferation is a rational target for disruption as a therapy against cancer. Some of the cancer cells induced to proliferate after primary tumor removal are cancer stem cells. The transient proliferative response in the residual cancer cell population is a highly choreographed response to tissue injury and facilitates the rapid regrowth of the cancerous tumor after surgery.

Considering the conserved nature of a tissue repair response induced by tissue damage, any cancer treatment, including radiation therapy or chemotherapy, are predicted to trigger a similar systemic tissue repair response that will facilitate the repair of the tumor via activation of stem cell self-renewal pathways.

Furthermore, the tissue repair response triggered by tissue damage may facilitate a process known as partial EMT, or partial epithelial-mesenchymal transition. This process may trigger the dedifferentiation of more differentiated cells to a stem cell phenotype.

The regrowth of a cancerous tumor after a treatment that damages it is ultimately under the control of a residual cancer stem cell population. Accordingly, after any treatment that damages the tumor, a residual cancer population may fall under the influence of a distinct systemic tissue repair response that will facilitate proliferation of surviving cancer stem cells and the dedifferentiation of more differentiated cancer cells to a stem cell phenotype (together, referred to as cancer stem cell enrichment). These two processes are reflexively engaged to facilitate the repair/regrowth of the tumor, are believed to be linked at the molecular level via conserved signaling processes.

Given a systemic tissue repair response induced by treatments damaging a cancerous tumor, and its highly predictable temporal pattern of expression after tissue injury, in one aspect there is provided a novel approach to cancer therapy that targets distinct cytokines at the time they are released after cancer treatments to prevent stem cell self-renewal and induction of EMT, and by preventing this process from occurring, mitigating the subsequent development of a drug resistant residual cancer cell phenotype.

Further, the acute inflammatory events associated with wound healing are generally finished within the first 14 days after tissue injury and well before conventional adjuvant chemotherapy is started.

Seeding of Cancer Cells into Circulation and Attachment to Microvasculature

Surgical resection of a variety of primary tumours is associated with seeding of cancer cells into the circulation, the mechanisms for which include tumour manipulation and breakdown of normal tissue planes. Once in the circulation and under anchorage-independent conditions these cells must overcome the shear forces imposed by blood flow and adhere to the microvasculature endothelium in order to extravasate into a target tissue and establish a metastatic deposit. This process is likely much more efficient immediately after surgery, given the increased vascular permeability and upregulated expression of cell surface adhesion molecules triggered by the release of acute inflammatory mediators such as histamine and thrombin.

Furthermore, the seeding of cancer cells into the circulation can induce the release of distinct inflammatory cytokines and the expression of E-selectin, thus directly facilitating their own metastasis.

Tissue damage also triggers platelet activation through the release of inflammatory molecules such as histamine, TNF-α, TGF-beta and others. Once activated, platelets express P-selectin, a molecule that facilitates platelet aggregation and cancer cell adhesion to platelets, resulting in the formation of cancer cell-platelet aggregates that can also bind fibrin, form tumour microthrombi and bind to vessel walls. These turnout microthrombi have a high affinity for binding to endothelial cells, facilitating metastatic colonization of tissue.

Tumour Removal Triggers an Inflammatory Response that can Enrich a Residual Cancer Cell Population for Tumorigenic Cancer Stem Cells

Tissue damage predictably triggers tissue specific stem cell self-renewal and proliferation. As detailed in the Examples, tissue damage has been shown to release specific molecular triggers that are important in the induction of stem cell self-renewal pathways. The surgical removal of the primary tumour can trigger a similar conserved response in a residual cancer stem cell population secondary to the release of tissue repair molecules that can trigger cancer stem cell self-renewal and proliferation.

Accordingly, targeting cancer stem cells in the immediate post-operative period using conventional chemotherapeutic agents, or novel agents targeting cancer cells may be more successful if they are actively undergoing self-renewal and proliferation. Furthermore, cancer stem cells may be particularly vulnerable to medical therapies used during the perioperative period, that can disrupt the formation and growth of nascent micrometastatic environments essential to cancer stem cell signalling and survival.

Besides the induction of residual cancer stem cell self-renewal as a result of the release of distinct “tissue damage” molecular signals after surgery, there is another complementary mechanism that may contribute to stem cell enrichment during the perioperative period. Differentiated cancer cells can de-differentiate to a stem/progenitor cell phenotype after passage through an EMT or epithelial-mesenchymal transition. The acute wound healing response triggered by primary tumour removal may be capable of inducing both residual cancer stem cell self-renewal and the de-differentiation of more differentiated cancer cells towards a cancer stem cell phenotype via EMT pathways. Indeed, tissue damage can trigger EMT in normal tissues as a highly-conserved tissue repair response.

If the inflammatory events after surgical removal of a primary tumour facilitate stem cell enrichment of a residual cancer cell population as a highly-conserved response to tissue (tumour) damage, treatment delays after surgery may be catastrophic given the relative resistance of cancer stem cells to conventional medical therapies. Adjuvant medical therapies must be started rapidly after the primary tumour has been removed to limit stem cell enrichment of any residual cancer cell population. The conventional treatment strategy universally followed worldwide of delaying treatment after surgical removal of a primary tumor to allow for surgical wound healing compromises our ability to eradicate a residual cancer cell population and fosters treatment failures and unnecessary deaths from otherwise curable malignancies.

The wound healing response triggered by surgical removal of the primary tumour acts on a residual cancer cell population during the perioperative period in ways that can facilitate haematogenous metastasis, micrometastatic formation and growth, as well as cancer stem cell enrichment of a surviving cancer cell population, interfering with the subsequent ability of adjuvant chemotherapy to eradicate residual disease. Disrupting the wound healing response during the perioperative period can enhance the ability to eradicate a residual cancer cell population after surgery and improve the ability to cure cancer.

Below are discussed therapies that can be employed alone or in combination in the adjuvant setting to disrupt the wound healing response after surgical removal of a primary tumour with the goal of facilitating the eradication of a residual cancer cell population.

### Targeting Cancer Stem Cell Self-Renewal and Epithelial-Mesenchymal Transition During the Perioperative Period

Surgical removal of the primary tumour triggers the release of inflammatory molecules that can induce both residual cancer stem cell self-renewal and the dedifferentiation of more differentiated cancer cells towards a stem cell phenotype via EMT pathways. Thus, a residual cancer cell population after surgery will rapidly become more treatment resistant the longer treatment is delayed as it becomes enriched for cancer stem cells under the influence of these inflammatory molecules. Targeting a residual cancer cell population rapidly after surgery using conventional chemotherapeutic agents will interfere with this process and limit stem cell repopulation. The specific molecular signals released after surgical removal of a primary tumour capable of inducing stem cell enrichment, may form the basis of targeted treatments against these signalling molecules at the time they are released.

An initial concern raised by using drugs that disrupt wound healing pathways during the perioperative period is one of safety, that this may increase wound healing or bleeding complications. Reassurance can be gained from studies of coronary artery bypass surgery, where the early use of aspirin resulted in superior survival compared to no aspirin, without significant haemorrhage or wound healing complications. Similarly a meta-analysis of 33 studies of prophylactic low or intermediate dose low molecular weight heparins (LMWHs) found that in general surgical patients had a low rate of bleeding complications requiring discontinuation of prophylaxis (2%) or further surgery (<1%).

### Improving Therapeutic Effectiveness Against Cancer Stem Cells by Employing Anti-Cancer Therapies at Time of Predicted Stem Cell Cycling/EMT Induction

The stem-cell enrichment associated with the up-regulation of distinct cytokines provides an alternate treatment strategy, which form a further aspect of the present invention, namely the targeting of cancer stem at the time they are undergoing stem cell cycling/EMT induction.

Curative cancer treatment may require that cancer stem cells be eradicated.

This cancer cell subpopulation has been shown to be very resistant to conventional anti-cancer therapies, including chemotherapy and radiation therapy. There are several reasons cancer stem cells are relatively resistant to conventional medical therapies. One mechanism of protection is via the adenosine triphosphate-binding cassette (ABC) transporters that are heavily expressed on the stem cell population. Without being limited to theory, these ABC efflux pumps can interfere with the entry of therapeutic agents into the cell. Cancer stem cells also appear to be more resistant to conventional medical therapies and radiation therapies because of heightened DNA repair mechanisms and a lowered propensity to undergo apoptosis, or programmed cell death, upon being damaged.

Without being limited to theory, among the reasons that cancer stem cells may possess high level resistance to conventional medical treatments, including chemotherapy and radiation therapy, is that these cells are often not dividing or dormant. This may be important from a therapeutic standpoint in that the anti-cancer therapies that are employed may require that the cell be actively dividing in order for the therapy to be effective. For example, and without being limiting, chemotherapy may kill cancer cells by disrupting the fidelity of the replication process, setting in motion apoptosis or programmed cell death pathways. However, if the cell is not dividing, these drugs may prove less effective at damaging the DNA molecule and triggering apoptosis and cell death.

For example, leukemic cancer stem cells, normally chemoresistant, may be sensitive to conventional chemotherapeutic agents when manipulated to undergo cell division. This suggests that dormancy, and not necessarily the multi-drug resistant phenotype, may be the key reason for therapeutic resistance of cancer stem cells to anti-cancer therapies.

In some embodiments, for example, the cancer may be leukemia, lung cancer, prostate cancer, colorectal cancer, breast cancer, or ovarian cancer.

The effectiveness of an anti-cancer treatment such as a chemotherapeutic drug may, for example, be time and concentration dependent. This may be due to the drug being removed from the body through physiological processes such as hepatic or renal clearance. The time frame a given drug dosage is effective may vary, but in respect of certain therapeutics, may not be longer than 24 hours, requiring additional dosages over time to maintain therapeutically effective concentrations in the patient undergoing treatment. As some chemotherapeutics may have a narrow therapeutic index (i.e., dose limiting toxicity may be found at levels necessary for therapeutic effectiveness), daily dosing of chemotherapy may not possible over an extended period of time.

Additionally, as cancer stem cells may often be dormant or non-proliferating, anti-cancer therapies may be ineffective unless they are employed at the specific time frame cancer stem cells are proliferating or are in EMI′.

Therefore, cancer stem cells, which may be treatment-resistant when dormant or not proliferating, may be susceptible to conventional cancer therapies upon the cancer stem cells entering into a proliferative, cycling or EMT phase.

In some embodiments, there is disclosed a method of inhibiting metastasis or recurrence of a cancer in a patient after a primary treatment of the patient comprising administering a therapeutically effective amount of a cancer therapy targeted towards a population of proliferating cancer stem cells in the patient. In some embodiments, the primary treatment induces the population of cancer stem cells to proliferate, self-renew or enter EMT.

In some embodiments, there is provided a method of treating a cancer in a patient comprising administering a treatment that induces a population of cancer stems cells in the patient to proliferate; and administering a therapeutically effective amount of a cancer therapy targeted towards the population of proliferating cancer stem cells. In some embodiments, for example, the treatment that induces the population of cancer stem cells to proliferate comprises administering a primary therapy to the patient.

In some embodiments, for example, the treatment that induces the population of cancer stem cells to proliferate comprises administering a composition to the patient. In some embodiments, for example, the composition comprises at least one growth factor that causes cancer stems cells to proliferate or self-renew. In some embodiments, the composition further comprises at least one pharmaceutically acceptable carrier. In some embodiments, for example, inducing the population of cancer stem cells to proliferate comprises the transition of cancer stem cells from a dormant or non-cycling phase to a proliferative phase, self-renewal phase or EMT phase.

The inventor has discovered a highly conserved signalling pattern upregulated at a distinct time period after surgical removal that will foster stem cell enrichment of a residual cancer cell population within 72-96 hours of surgical removal of the primary tumor. Given this reproducible observation, using chemotherapy at the time the residual cancer cells are likely to be proliferating, i.e. within 24-48 hours of surgical removal of the primary tumour, will prove far more effective at killing off a residual cancer cell population than the standard therapy currently employed much later in time.

In some embodiments, the cancer therapy is administered at the time of primary therapy or administering the treatment that induces the population of cancer stem cells in the patient to proliferate; or within 6 hours, 12 hours, 18 hours, 24 hours, 30 hours, 36 hours, 42 hours, 48 hours, 54 hours, 60 hours, 66 hours, 72 hours, 78 hours, 84 hours, 90 hours, or 96 hours after primary treatment or administering the treatment that induces the population of cancer stems cells in the patient to proliferate. In other embodiments, within 120 hours or within 1 week. In some embodiments, for example, the cancer therapy is administered before about 24 hours after the primary treatment or administering the treatment that induces the population of cancer stems cells in the patient to proliferate. In some embodiments, for example, the cancer therapy is administered from about 24 to about 96 hours after the primary therapy or administering the treatment that induces the population of cancer stems cells in the patient to proliferate. In some embodiments, the method increases the efficacy of cancer therapies.

In some embodiments, for example, the cancer therapy may be one or more of endocrine therapy, chemotherapy, hormone therapy, gene therapy, thermal therapy, ultrasound therapy, radiation therapy, immunotherapy, small molecule inhibitor therapy and targeted biologic therapies such as monoclonal antibodies, RNA interference (including but not limited to microRNA and small interfering RNA).

In some embodiments, for example, these therapies may be effective in inducing stem cell apoptosis if used against cancer stem cells when said cancer stem cells are proliferating. In some embodiments, for example, the cancer therapy comprises one or more compounds, compositions or other therapeutics that are known in the art as being cytotoxic specifically for proliferating cancer cells.

In some embodiments, for example, treatment may include administration of chemotherapeutic agents that target the machinery of cell division, including but not limited to alkylators (cyclophosphamide/cisplatin/melphalan); topoisomerase inhibitors 1 and 2 (doxorubicin/irinotecan/etoposide/topotecan); taxanes (docetaxel/paclitaxel/abraxane); vinca alkaloids (vincristine/vinblastine); antimetablolites (5-FU/Gemcitabine/Cytarabine/Pemetrexed).

In some embodiments, for example, the cancer therapy comprises nanoparticle-mediated thermal therapy. In some embodiments, for example, the nanoparticle mediated thermal therapy comprises administration of antibodies specific for the population of proliferating cancer stem cells, wherein the antibodies are bound or conjugated to a nanoparticle that may be selectively induced to increase in temperature using a stimulus. Without being bound by theory, upon administration of the antibody-bound nanoparticle, the antibodies may bind to some or all of the proliferating cancer cells. Subsequently, the nanoparticles may be selectively induced to increase in temperature using the stimulus, and the increase in temperature of the nanoparticles bound to the proliferative cancer stem cells via the antibody causes apoptosis, cell death, and/or inhibition of proliferation in the population of proliferating cancer stem cells. In some embodiments, for example, the nanoparticle may comprise a biologically compatible gold nanorod. In some embodiments, for example, the stimulus may be one of radiation, vibratory stimulus, high frequency microwave, high frequency radio wave, electromagnetic radiation, or other biologically compatible stimulus capable of inducing the increase in temperature of the nanoparticles known to those skilled in the art. In some embodiments, the antibody may be any of the antibodies described above that are specific for cancer stem cells.

Predicting when cancer stem cells may be proliferating requires understanding the events common to the highly conserved process of wound healing to provide insight into normal stem cell behavior that can be extrapolated to the behavior of cancer stem cells. For example, in vivo, damage to normal tissue triggers tissue specific stein cell self-renewal, proliferation and/or EMT. The time frame for this increase in stem cell proliferation or induction into EMT is approximately 24 hours after the tissue damage. The clonal proliferation of the stem cell compartment in response to tissue damage follows a temporal pattern of expression that is transient and lasts for about 48-72 hours.

It has been shown, for example, that 24 hours after removal of a variety of different primary tumors in animal models, there is an increase in the proliferation of cancer cells in other metastases in the body. This growth factor or growth factors were shown to cause cells to convert from a non-cycling phase (or dormant phase) to a proliferative phase, resulting in reductions in tumor doubling times and increased growth rates of metastases. This increase in proliferation lasts for about 72 hours.

As detailed in Example 2, it is found that HGF is upregulated after tumour removal. As further detailed in Example 5, it is found that HGF in isolation renders cancer cells highly sensitive to the effects of chemotherapeutic agents. In one embodiment there is provided, a method of treating cancer stem cells by blocking the influence of specific cytokines, including IL-6, PGE-2, PGF, MCP-1 and MMP-9 at the time they are upregulated (ligand/receptor/downstream actors) while allowing the predicted upregulation of HGF to occur after tumor removal, and applying a cytotoxic treatment. Blocking the upregulation of IL-6, PGE-2, PGF, MCP-1 and/or MMP-9 while allowing the upregulation of HGF may cause a surviving cancer stem cell population to become very vulnerable to the effects of cytotoxic chemotherapy.

In an embodiment, there is provided a method of inhibiting metastasis or recurrence of a cancer in a patient after a primary treatment of the patient comprising: obtaining a patient sample; determining a cancer stem cell proliferation profile for the patient; comparing the cancer stem cell proliferation profile to a reference cancer stem cell proliferation profile; and administering a therapeutically effective amount of a cancer therapy targeted towards a population of proliferating cancer stem cells in the patient if the cancer stem cell proliferation profile is greater than or equal to the reference cancer stem cell proliferation profile.

In an embodiment, determining the cancer stem cell proliferation profile comprises determining a concentration of markers in the patient sample indicative of cancer stem cell proliferation. In some embodiments, for example, the markers comprise cellular receptors that are indicative of cancer stem cell proliferation or self-renewal. In some embodiments, the markers comprise cytokines that are indicative of cancer stem cell proliferation, self-renewal or shift into EMT. In some embodiments, the cytokines are selected from TGF-beta, IL-6, HGF, PGE-2, PGF, PDGF-BB, MCP-1 and MMP-9. In one embodiment, IL-6 and HGF and, optionally, one or more of TGF-beta, PGE-2, PGF, PDGF-BB, MCP-1 and MMP-9; in one embodiment, IL-6, HGF, TGF-beta, PGE-2, and PDGF-BB.

In a further embodiment, determining the cancer stem cell proliferation profile comprises determining the extent of proliferation of cancer stem cells in the patient sample. In some embodiments, for example, proliferation may be determined by running the sample in a fluorescence activated cell sorter (FACS) and measuring the fraction of cells proliferating by measuring DNA content as a surrogate for cells undergoing cellular division. In some embodiments, for example, circulating cancer cells retrieved from a patient after surgery are cultured, and the retrieved cells' ability to be maintained in culture and to be passaged may be used to identify the extent of the stem cells within the circulating tumor cell population, as it is the stem cell fraction cells that would enable the persistence of the culture line.

In some embodiments, the patient sample is obtained at one or more of the group consisting of: prior to primary treatment, at the time of primary treatment, within 1 hour after primary treatment, within 6 hours after primary treatment, within 12 hours after primary treatment, within 18 hours after primary treatment, within 24 hours after primary treatment, within 30 hours after primary treatment, within 36 hours after primary treatment, within 42 hours after primary treatment, within 48 hours after primary treatment, within 54 hours after primary treatment, within 60 hours after primary treatment, within 66 hours after primary treatment, within 72 hours after primary treatment, within 78 hours after primary treatment, within 84 hours after primary treatment, within 90 hours after primary treatment and within 96 hours after primary treatment. In other embodiments, within 120 hours or within 1 week.

In some embodiments, there is disclosed a kit for use in determining the extent of proliferation of a population of cancer stem cells, the kit comprising: one or more antibodies specific for a receptor on the cancer stem cells that is upregulated in response to cancer stem cell proliferation. In some embodiments, there is disclosed a kit for use in determining the extent of proliferation of a population of cancer stem cells, the kit comprising: one or more antibodies specific for a cytokines that are upregulated in response to cancer stem cell proliferation

In some embodiments, for example, the patient sample comprises a tumour sample of the patient. In some embodiments, for example, the patient sample comprises a metastatic tumour sample of the patient. In some embodiments, for example, the patient sample is a tissue sample local to the site of a patient tumour or local to the site where the tumour resided prior to a primary therapy. In some embodiments, the patient sample is a tissue sample distal to the site of the tumour or where the tumour resided prior to primary therapy. In some embodiments, the patient sample is a blood sample.

In some embodiments, the cancer therapy is administered at the time of primary therapy or administering the treatment that induces the population of cancer stem cells in the patient to proliferate; or within 6 hours, 12 hours, 18 hours, 24 hours, 30 hours, 36 hours, 42 hours, 48 hours, 54 hours, 60 hours, 66 hours, 72 hours, 78 hours, 84 hours, 90 hours, or 96 hours after primary treatment or administering the treatment that induces the population of cancer stems cells in the patient to proliferate. In some embodiments, for example, the cancer therapy is administered before about 24 hours after the primary treatment or administering the treatment that induces the population of cancer stems cells in the patient to proliferate. In some embodiments, for example, the cancer therapy is administered from about 24 to about 96 hours after the primary therapy or administering the treatment that induces the population of cancer stems cells in the patient to proliferate. In some embodiments, the method increases the efficacy of cancer therapies.

In some embodiments, for example, the cancer is metastatic. In some embodiments, the primary treatment may be for treatment of a cancer that metastasized.

In some embodiments, administering the cancer therapy targeted towards the population of proliferating cancer stem cells induces apoptosis in the population of cancer stem cells. In some embodiments, administering the cancer therapy targeted towards the population of proliferating cancer stem cells inhibits proliferation or self-renewal of the population of cancer stem cells. In some embodiments, for example administering the cancer therapy targeted towards the population of proliferating cancer stem cells inhibits or prevents metastasis or cancer recurrence.

### Therapeutic Targeting of Wound Healing Pathways Perioperatively

In certain aspects, other wound healing pathways described below may be targeted perioperatively as part of methods of the present invention.

### Inhibiting Immune Clearance of Circulating Tumour Cells

After major surgery, marked impairment in the immune response is noted, characterized by reduced lymphocyte proliferation to mitogen challenge, a fall in numbers of CD4+ T cells, B cells and NK cells and a fall in the CD4:CD8 T cell ratio. The release of several acute inflammatory mediators after surgery appears to be the predominant mechanism inducing immunosuppression. Furthermore immunosuppression appears to be important to the formation of premetastatic niches suitable for tissue colonization by circulating cancer cells.

The body's immune defences, particularly cell-mediated immunity (CMI)—natural killer [NK] cells, macrophages, cytotoxic T lymphocytes—may be very effective at eliminating circulating cancer cells and maintaining CMI helps to minimize metastasis during the perioperative period. Accordingly, in one embodiment methods according to the prevent invention include cell-mediated immunotherapy.

### The Perioperative Period May be Conducive to the Formation of Micrometastases

Tissue damage triggers the egress from the bone marrow of a variety of cells to sites of tissue damage, including haematopoietic progenitor cells, where they facilitate tissue repair. Cancer cells can induce a similar tissue repair response to facilitate their metastatic colonization of tissue. Growth factors derived from implanted cancer cells are able to trigger the rapid formation (14 days after implantation) of “premetastatic niches” to which the cancer cells can subsequently bind to and develop into viable metastatic tumours. These niches can be imagined as scaffolds to which the circulating cancer cells could bind, creating a permissive tissue environment for micrometastasis formation.

The key event triggering the formation of these niches involves the mobilization of bone marrow derived progenitor cells to distinct premetastatic tissue sites. The tumour-derived molecules that have been implicated in directing this process in different cancers include VEGF, placental growth factor, TGF-beta, TNF-α lysyl oxidase (LOX), and versican.

Primary tumour hypoxia may lead to the formation of premetastatic niches. Hypoxia-inducible factor (HIF)-dependent expression of LOX and LOX-like proteins can alter the extracellular matrix (ECM) in premetastatic niches to facilitate subsequent cancer cell colonization in pre-metastatic organs. LOX, secreted by hypoxic tumour cells under the influence of HIE, localizes with fibronectin in premetastatic niches and cross-links collagen IV in the basement membrane to promote the adhesion of CD11b+ bone marrow derived haematopoietic progenitor cells critical to niche formation.

Given the link between tumour hypoxia and induction of premetastatic niche formation, events after surgical removal of the primary tumour may create a favourable environment for niche formation. Major surgery is known to induce tissue hypoxia perioperatively as well as significant elevation in the release of a variety of angiogenic molecules implicated in premetastatic niche formation. Furthermore, tissue damage after primary tumour removal triggers the mobilization of bone marrow-derived haematopoietic progenitor cells essential to the formation of premetastatic niches. Thus the perioperative period is an ideal time frame for the formation of premetastatic niches given that the signalling molecules essential for their formation will be upregulated during this time secondary to the inflammatory processes associated with wound healing.

Furthermore, the ability to trigger the production of premetastatic niches through the elaboration of specific signalling molecules may reside in distinct cancer cell populations, such as cancer stem cells or cancer cells that have undergone epithelial-mesenchymal transition (EMT) and/or are capable of anchorage-independent growth. As discussed, the inflammatory events triggered by surgical removal of the primary tumour are believed to facilitate the enrichment of any surviving cancer cell population for these specialized types of cancer cells.

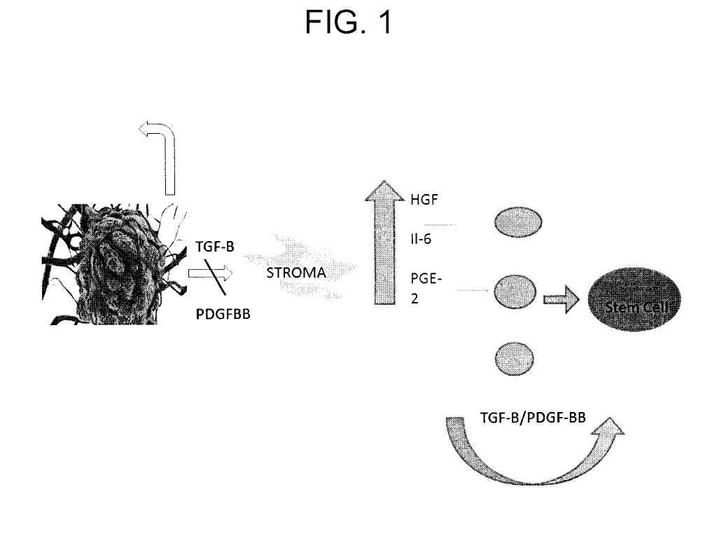

TGF-beta can down regulate the expression of HGF. Based upon an understanding of the critical roles played by HGF during the normal process of wound healing, primary tumor removal and the consequent transient reduction in the levels of TGF-beta could upregulate HGF transiently, facilitating the development of the pre-metastatic niche. This process can also be amplified by disruption of the primary tumor during surgery, resulting in the release of viable tumor cells into the circulation, which, when freed of the autocrine and paracrine influence of TGF-beta, increase the of expression of growth factors such as PDGF, B-FGF, and/or IL-1 which can act on stromal fibroblasts to enhance expression of HGF and activate the normal wound healing process. At the invasion front itself and within the primary tumor, TGF-beta would likely play the more dominant role, and the stromal expression levels of HGF would correspondingly be lower during this stage of the process. FIG. 1 depicts the expression of cytokines after tumour removal. Tumour removal is shown to lead to a drop in TGF-beta and PDGFBB levels acutely, which triggers increased stromal secretion of HGF, PGE-2 and IL-6. This increase in these cytokines combined with a rapid return of TGF-beta and PDGFBB levels to baseline or above will facilitate stem cell enrichment of a residual cancer cell population. This molecular switch appears to be particularly important in triggering the process leading to stem cell enrichment in a residual cancer cell population.

### Primary Tumour Removal Triggers the Enhanced Growth of Existing Micrometastases

Micrometastatic deposits require angiogenesis for growth beyond 2 mm. After surgical removal of the primary tumour markers of angiogenesis are upregulated and induce accelerated growth of existing micrometastases. Fisher and colleagues discovered that surgical removal of primary tumours in animal models triggered the proliferation of residual cancer cells in existing macroscopic metastases, resulting in the transient, rapid growth of metastatic tumours. Interestingly, surgery on non-tumour tissue did not induce this pattern; the primary tumour had to be removed. The present inventor is accordingly of the view, supported by the examples, that primary tumour removal triggers a distinct systemic response from that triggered by surgical trauma per se.

Accordingly, other targets for cancer therapy are;

1. Platelet Activation and Platelet-Tumour cell interactions
2. Tumour cell adhesion to activated endothelium

### 3. Immunosuppression

4. Angiogenesis/Premetastatic niche formation

Therapeutic options to disrupt each of these targets during the perioperative period are available, including agents, or classes of agents, currently in clinical use, many of which are not currently considered anticancer drugs (Table 1).

TABLE 1
Examples of currently-available agents that could potentially
improve cancer outcomes by targeting the wound healing pathways
in the perioperative period (grouped by therapeutic target)

| Therapeutic target | Mechanism disrupted | Agents |
|---|---|---|
| Platelet activation | Platelet and coagulation | NSAIDs, H2RAs, |
| and cancer cell- | cascade activation; | prostacyclin analogues, |
| platelet interac- | P-selectin expression/ | pentoxyfylline, heparins |
| tions. | interaction with ligands | |
| Tumour cell- | P- and E-selectin | H2RAs, statins, |
| endothelium | expression, tumour cell | thalidomide, |
| adhesion | adhesion to selectins and | pentoxyfylline, heparins |
| | integrins | |
| Perioperative | Immunosuppression from | H2RAs, heparins |
| immunosuppres- | acute inflammatory | |
| sion | mediators | |
| Angiogenesis/ | Angiogenesis; fibroblasts, | Angiogenesis inhibitors, |
| premetastatic | endothelial cells, | NSAIDs, thalidomide, |
| niche formation. | platelets, bone marrow- | H2RAs, heparins, |
| | derived haematopoietic | cytotoxic chemotherapy |
| | progenitor cells | |
| | | NSAIDs, heparins, |
| | | cytotoxic chemotherapy |

Key:
NSAIDs: non-steroidal anti-inflammatory drugs;
H2RAs: histamine type-2 receptor antagonists;
EMT: epithelial-mesenchymal transition;
TKI: tyrosine kinase inhibitor

In certain embodiments, agents inhibit multiple targets.

### Targeting Platelet Activation and Platelet-Tumour Cell Microthrombi

Inhibitors of platelet aggregation and activation have potential to interfere with tumour cell-platelet interactions. Non-steroidal anti-inflammatory drugs (NSAIDs), which inhibit cyclooxygenase-1 (COX-1), thereby reducing platelet release of thromboxane and subsequent platelet activation, are the most commonly-used drugs affecting this pathway. Accordingly, these drugs may be used to prevent, and reverse, platelet activation and thus platelet-tumour cell adhesion during the perioperative period.

However the use of NSAIDs may have marked benefits in a much larger proportion of patients. Preliminary clinical evidence to support this comes from a retrospective analysis of breast cancer relapse in patients treated by a single surgeon; those who received perioperative ketorolac had a 5-fold reduction in relapses in the 9-18 month period of greatest risk, compared to those who received other analgesics, or NSAIDs post-operatively. However this putative benefit of NSAIDs may not be seen in patients taking them prior to diagnosis of cancer, given the observation that those who developed colorectal cancer while taking aspirin derived no survival advantage from it, whereas those who took it exclusively afterwards did so, suggesting that tumours that had developed while exposed to NSAIDs were resistant to their blockade of various pathways.

Use of prostacyclin, among the most potent inhibitors of platelet activation and aggregation, resulted in a reduction in pulmonary metastases and a complete elimination of hepatic metastases in the B16 amelanotic melanoma model. Prostacyclin analogues, such as iloprost and cicaprost, have also shown significant ability to inhibit metastasis formation.

Inhibitors of platelet activation through inhibition of P-selectin include statins and histamine type 2 receptor antagonists (H2RAs), though the effects of the latter appear to be weaker than for NSAIDs. Heparins can interfere with the interaction between tumour cells, platelets, and endothelial cells by disrupting tumour cell binding to P and E-selectin and heparin administered prior to injection of tumour cells can dramatically interfere with haematogenous metastasis and micrometastasis formation.

### Targeting Perioperative Immunosuppression

Major surgery induces immunosuppression for several days or weeks, in part due to systemic release of inflammatory cytokines and histamine. Blockade of H₂ receptors inhibits this effect, resulting in enhancement of immune function, including cell-mediated immunity, antigen-presenting activity of dendritic cells and antitumour activity of natural killer (NK) cells. Heparins may also be helpful at this time as they prevent thrombin from concealing tumour cells. H2RAs also enhance immune response to tumours, with increased tumour-infiltrating and peritumoral lymphocytes, both of which have favourable prognostic significance.

### Targeting Angiogenesis/Premetastatic Niche Formation

Targeting angiogenesis is a logical treatment strategy, particularly during the perioperative period. In experiments by Kaplan, disruption of niche formation using bevacizumab prevented metastatic colonization. The rationale for the use of such drugs perioperatively is based on the assumption that this period of time would be ripe for the development of premetastatic niches and formation of micrometastases.

If premetastatic niche development was already well advanced under the influence of the primary tumour prior to surgery trying to disrupt premetastatic niche formation during the perioperative period would not necessarily prove effective. Evidence to support this comes from a recent NSABP trial in which patients with colon cancer were randomized to receive bevacizumab in addition to adjuvant chemotherapy. The group receiving the antiangiogenic agent had delayed relapse during the year on treatment but then caught up with the chemotherapy-only group, with no net benefit in overall survival. However bevacizumab was not started until 29-50 days post-operatively, a stage at which it was unlikely to affect formation of premetastatic niches or their interaction with circulating tumour cells during the perioperative period; instead bevacizumab would be inhibiting angiogenesis around established metastases. Administration of bevacizumab early in the post-operative period (within 5-10 days) would be beneficial.

Other agents that could be used perioperatively to reduce angiogenesis include LMWHs, H2RAs (through inhibiting histamine's angiogenic effects), NSAIDs and receptor tyrosine kinase inhibitors (TKIs) that disrupt angiogenic signalling.

Both NSAIDs and LMWHs can disrupt the signalling process implicated in the formation of premetastatic niches and micrometastases. The chemotactic chemokine SDF-1 (stromal derived factor) serves as a homing molecule for both CXCR4 expressing cancer cells as well as the hematopoietic progenitor cells implicated in premetastatic niche formation. NSAIDS have been shown to downregulate the expression of SDF-1, while LMWH was able to disrupt the interaction of the chemokine receptor CXCR4 and its ligand CXCL12, disrupting the formation of hepatic metastases in colon cancer.

Premetastatic niche formation and growth of existing micrometastases could also be disrupted using conventional chemotherapeutic agents rapidly after surgery. Conventional chemotherapy is effective at not only killing rapidly dividing cancer cells, it can kill other dividing cells, including those cells that may be contributing to the survival of a residual cancer cell population during the perioperative period. These cells include platelets as well as the cells implicated in premetastatic niche formation, including endothelial cells, fibroblasts, and bone marrow derived haematopoietic progenitors. In fact, given that conventional chemotherapy is largely ineffective against cancer stem cells and almost always incapable of eradicating clinically evident metastatic disease, the effectiveness of adjuvant chemotherapy may derive in part from its ability to destroy these normal cell populations essential to niche formation and cancer stem cell survival. The effectiveness of adjuvant chemotherapy may be magnified perioperatively if it is started early enough to disrupt the formation and growth of premetastatic niches critical to stem cell survival.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

All documents referenced herein are incorporated by reference, however, it should be appreciated that any patent, publication, or other disclosure material, in whole or in part, that is incorporated by reference herein is incorporated only to the extent that the incorporated material does not conflict with definitions, statements, or other disclosure material set forth in this disclosure. As such, and to the extent necessary, the disclosure as explicitly set forth herein supersedes any conflicting material incorporated herein by reference.

The embodiments of the invention described above are intended to be exemplary only. The scope of the invention is therefore intended to be limited solely by the scope of the appended claims.

### EXAMPLES

### Example 1

Delaying Chemotherapy after Tumour Removal Will Lead to Increase in Cancer Stem Cells

After potentially curative surgical removal of a primary tumour and no radiographic evidence of metastatic disease any surviving cancer cells will be within immature tissue micrometastatic deposits or the circulation. The surgical removal of a variety of different tumours in animal models was shown to have a growth stimulating effect on cancer cells within evident metastatic deposits in seminal studies by Fisher and colleagues.

The increase in proliferation was noted at approximately 24 hours after surgery and lasted for approximately five to seven days. This increase varied from cancer type, but all cancers showed a significant increase from baseline after tumour removal. After this transient increase, cancer cell proliferation fell to the levels noted prior to surgical removal of the primary tumour.

Stem cells, whether cancer stem cells or tissue specific stem cells, are generally dormant or non-proliferating. When they are proliferating, they can divide asymmetrically (one stem cell and one daughter cell) or symmetrically (two stem cells).

After tumour removal an increased number of cancer cells in extant metastatic foci transited into a cycling mode. The percentage of cells transiting into a cycling mode was not increased by administering higher doses of serum from animals with removed tumours. The present inventor is therefore of the view that a fixed number of cells in these foci are able to respond to the growth factor of factor(s) released after tumour removal.

Both normal and neoplastic cells can spontaneously dedifferentiate to a stem cell phenotype. This phenotypic transformation permits these cells to behave like stem cells. In the case of cancer stem cells, these behaviors include resistance to conventional medical treatments and a heightened ability to metastasize and colonize tissue. Neoplastic cells appear particularly susceptible to this de-differentiation process as a result of the genetic instability inherent in the neoplastic phenotype.

Cells that have been experimentally manipulated to undergo the process of epithelial-mesenchymal transition or EMT can also revert to a stem cell phenotype. The process of wound healing/tissue repair has been shown to release a number of growth factors that can trigger an EMT like process in normal and neoplastic epithelial cell populations under various physiologic states. Because of the known association between partial EMT induction and wound healing, systemic tissue repair response triggered by any cancer treatment, including primary tumour removal, will accelerate the observed spontaneous rate of dedifferentiation of a cancer cell population to a stem cell phenotype. Both chemotherapy treatment and radiation treatment will foster stem cell enrichment of a residual cancer cell population rapidly after treatment. The enrichment for cancer stem cells in response to chemotherapy or radiation therapy is the result of tissue damage signals inducing both stem cell proliferation and the induction of a partial EMT in a residual cancer cell population. A similar response will be triggered after the surgical removal of the primary tumour.

The present inventor modelled the growth of a residual cancer cell population after surgery.

Assume that the cancer cell population remaining after surgery is 100,000 cancer cells, which is the number of cells estimated to reside in a tiny one millimetre micrometastatic deposit.

N0=Population of cancer cells remaining after surgical removal of the primary tumour (One hundred thousand cancer cells).

This cancer cell population will come under the influence of the inflammatory events triggered by primary tumour removal as discussed above. In the experiments by Fisher the fraction of the cancer cell population transiting into a cycling mode reached approximately 40% in some of the tumour types studied. We denote this as rᵢ=40%. Cell cycle time for cancer cells undergoing cell division is approximately 24 hours. The duration of time for the acute increase in proliferation was approximately six days (t*=6; days 1-6). We denote the total number of cancer cells at time t as N (t), note this includes all the cancer cells, including the stem cell like or non-stem cell like cancer cells. Hence we have the following equation for the growth of a residual cancer cell population during the initial six days following surgical resection of the primary tumour:

N(t)=N₀(1+r₁)^{t}, t≤t*

From day 7 to day 30 the rate of proliferation can be expected to fall to normal values for micrometastases. It is difficult to precisely quantify this rate, which varies according to tumour type, but an average estimate would be a tumour doubling time of 30 days and therefore the fraction of cells proliferating during this time would be approximately 2.33% (r₂=0.0233). Assuming the same cycle time a similar equation can be used to estimate the growth of this cellular population from day 7 to day number 30.

N(t)=N₆(1+r₂)^{t-6}, t>t*

Second, we model the growth of the stem cell fraction. We assume symmetrical division of a residual cancer stem cell population rapidly after surgery based on the observation that tissue damage triggers symmetrical stem cell division of normal tissue stem cells acutely after tissue injury. We choose day one to day six for symmetrical stem cell division based on the increased proliferation period noted above. Similarly, we assume asymmetrical stem cell division from day seven to day thirty as the proliferation of an extant cancer cell population falls to normal levels after the initial acute increase noted after surgery.

Finally, the rate of dedifferentiation of more differentiated cancer cells to a cancer stem cell phenotype can be approximated. The spontaneous conversion rate of the non-neoplastic population was 0.0170 per cellular division, while it was much higher in the neoplastic population of 0.0025. For the sake of our model we will use the lower conversion rate of 0.0170 from day seven to day thirty and the higher conversion rate of 0.0025 per cell division acutely after surgery (six days).

Given these assumptions, the growth of a residual cancer cell population after surgery can be expressed mathematically within these two simultaneous equations:

N′(t)=N′(t−1)(1+r₁)+μ₁[N(t−1)−N′(t−1)], t≤t*

N′(t)=N′(t−1)(1+r₂/2)+μ₂[N(t−1)−N′(t−1)] t>t*

(N′ (t) is the number of cancer stem cell at day t. The two terms in the equation are from self-renewal of stem cells and the dedifferentiation of more differentiated cancer cells to a stem cell phenotype; μ₁ 0.017 and μ₂=0.0025 are the conversion rates of more differentiated cancer cells to a stem cell phenotype during stage 1 (day 1 to 6) and stage 2 (day 7 to 30) respectively).

The increase in the stem cell fraction of the residual cancer cell population after surgery can be expressed as: F (t)=100 N′ (t)/N (t).

Similarly, the absolute increase in the number of cancer stem cells N′ (t) and more differentiated cancer cells N (t) is presented in Table 2.

TABLE 2

| | | N(t) − | | |
|---|---|---|---|---|
| Day | N′ (t) | N′(t) | N(t) | F(t) |
| Day 0 | 1000 | 99000 | 100000 | 1 |
| Day 1 | 1839 | 138161 | 140000 | 1.313071 |
| Day 3 | 4923 | 191077 | 196000 | 2.511413 |
| Day 4 | 10140 | 264260 | 274400 | 3.695203 |
| Day 5 | 18688 | 365472 | 384160 | 4.864618 |
| Day 6 | 32376 | 505447 | 537824 | 6.019833 |
| Day 7 | 53919 | 699034 | 752953 | 7.161021 |
| Day 10 | 62107 | 744879 | 806986 | 7.696139 |
| Day 15 | 77504 | 828290 | 905794 | 8.556441 |
| Day 20 | 95354 | 921346 | 1016700 | 9.378803 |
| Day 25 | 116000 | 1025186 | 1141186 | 10.1649 |
| Day 30 | 139828 | 1141085 | 1280913 | 10.91632 |

N′ (t) is equal to number of cancer stem cells and N (t) is equal to total number of cancer cells after surgery.

Given the assumptions of our model, an exponential increase in a residual cancer cell population for cancer stem cells will occur rapidly after surgical removal of the primary tumour and well before the time chemotherapy is now conventionally started.

Chemotherapy given rapidly after primary tumour removal could limit stem cell repopulation dramatically by killing off the bulk of the more differentiated cancer cells remaining after surgery. This would limit the number of cells capable of dedifferentiating to a stem cell phenotype, a process that is most responsible in our model for the increase in cancer stem cells after surgery.

Further, based on the model above, a residual cancer stem cell population is predicted to be actively proliferating within 24-48 hours of surgery under the influence of a systemic tissue repair response, thereby being more susceptible to many chemotherapy treatments.

The model predicts that a residual cancer stem cell population will grow exponentially during the time before the start conventional chemotherapy.

The rapid increase in treatment resistant cancer cells after surgery is due to a molecular plasticity inherent in cancer cells populations rendering them highly responsive to environmental perturbations induced by our clinical interventions timing of medical treatment after surgical removal of the primary tumour is critical for inhibiting disease recurrence and mestastasis. Optimization of the potentially curative medical treatment of cancer requires that treatment be started rapidly after the primary tumour is removed to prevent repopulation of drug resistant and metastatically competent cancer stem cells.

### Example 2

The Distinct Cytokine Response after Surgical Removal of Tumours

Specimens for cytokine testing were collected by aseptic technique into EDTA tubes. Specimens from surgical patients were collected at eight intervals—before surgery, after surgery (while the patient was in recovery), at 24 hours, 48 hours, 72 hours, 1 week, 2 weeks and 4 weeks after surgery.

EDTA samples were centrifuged within 30 minutes of collection, plasma was removed and then recentrifuged. Plasma was then aliquoted into cryotubes and stored in a −80° C. freezer. On the morning of testing, cryotubes containing an aliquot of plasma from designated patients were placed into the 4° C. refrigerator to thaw, then vortexed and recentrifuged for 5 minutes at 10,000 g. Testing was performed immediately after. Levels of various cytokines were measured at the indicated time points.

Results of tests performed using EMD Millipore kits were read on the Luminex 200 analyzer. This flow cytometer based instrument integrates key detection components, such as lasers, optics, advanced fluidics and high speed digital signal processors. The multiplex technology is capable of performing a variety of bioassays including immunoassays on the surface of fluorescent coded magnetic beads known as MagPlex™-C microspheres. Results are quantified based on fluorescent reporter signals.

The surgical removal of three different tumour types—Colorectal, Breast, Prostate—triggers a statistically significant decline after surgery in the levels of TGF-beta and PDGF-BB at twenty four (24) hours after surgery. This is followed by a rapid increase back to baseline or even increased levels of TGF-beta and PDGF-BB. This, in turn is associated with a reciprocal statistically significant upregulation in the levels of IL-6, HGF, MCP-1, MMP-9 and PGF. This upregulation occurs at approximately the 24 hour mark after surgery and tends back to baseline within approximately one week of surgery.

As furthered detailed in Example 3, applying these distinct cytokines/growth factors alone or in combination to cancer cells in culture at levels detectable in the serum of patients after surgery, either from established cancer cell lines or acquired from cultured circulating tumour cells, triggers an increase in the fraction of the cells that are proliferating; facilitates epithelial-mesenchymal transition; and demonstrates an increase in the stem cell fraction as compared to cell lines not exposed to these molecules. This can be taken as evidence that the upregulation in these distinct molecules after surgical removal of a primary tumour can facilitate an increase in a residual cancer cell population for cancer stem cells rapidly after surgical removal of a primary tumour and within one week of surgery. Blocking these upregulated molecules at the time they are predicted to be upregulated may prevent stem cell reproduction of a surviving cancer cell population after surgery.

Table 3 shows that IL-6 is significantly upregulated after surgery, and at Day 1 and Day 2 after surgery.

TABLE 3
significant upregulation of IL-6 after surgery, at Day 1 and Day 2 after surgery.

| IL-6 | Before | After | D 1 | D 2 | D 3 | D 7 | D 14 | D 28 |
|---|---|---|---|---|---|---|---|---|
| Patient 1 | 3.33 | 2.77 | 4.13 | 2.14 | 1.48 | 27.66 | 1.7 | 1.45 |
| Patient 2 | 2.21 | 28.97 | 46.71 | 28.64 | 30.86 | 38.56 | 29.18 | 12.19 |
| Patient 3 | 60.45 | 78.18 | 97.91 | 71.85 | 69.74 | 45.97 | 43.51 | 49.12 |
| Patient 4 | 7.73 | 5.29 | 8.71 | 6.27 | 4.09 | 3.99 | 4.37 | 4.06 |
| Patient 6 | 3.27 | 4.71 | | 7 | 4.79 | 5.11 | 20.83 | 1.96 |
| Patient 7 | 0.67 | 17.77 | 28.82 | 9.35 | 4.73 | 4.23 | 1.74 | 1.92 |
| Patient 8 | 7.29 | 12.35 | 60.27 | 26.16 | 10.97 | 33.81 | 102.1 | 9.61 |
| Patient 9 | 44.3 | 94.42 | 119.96 | 204.65 | 90.32 | 27.73 | 35.48 | 35.77 |
| Patient 10 | 2.48 | 1.88 | 4.3 | 2.8 | 2.59 | 1.85 | 2.25 | 1.33 |
| Patient 11 | 10.56 | 2.18 | 2.04 | 1.2 | 0.43 | 0.42 | 0.51 | 0.38 |
| Patient 13 | 21.25 | 47.33 | 129.83 | 25.7 | 9.17 | 12.05 | 4.93 | 24.87 |
| Patient 14 | 1.91 | 3.97 | 6.43 | 19.92 | 21.38 | 7.56 | 1.75 | 3.88 |
| Patient 15 | 0.44 | 23.96 | 35.72 | 21.74 | 10.94 | 4.9 | 0.84 | 0.77 |
| Patient 16 | 0.92 | 6.75 | 8.59 | 2.45 | 1.43 | 1.33 | 1.09 | 1.31 |
| Patient 17 | 31.85 | 22.26 | 7.01 | 6.47* | 6.17* | 41.88 | 27.82 | 14.23 |
| Patient 19 | 57.37 | 62.4 | 210.85 | 90.83 | 31.88 | 18.9 | 74.59 | 57.16 |
| Patient 20 | 1.82 | 5.06 | 6.52 | 6.76 | 3.66* | 3.12 | 1.67 | 1.67 |
| Patient 21 | 17.21 | 71.16 | 104.72 | 38.45 | 16.26 | 25.07 | 24.41 | 35.51 |
| Patient 22 | 8.89 | 48.77 | 49.85 | 31.68 | 44.45 | 34.89 | 12.62 | 9.07 |
| Patient 23 | 3.34 | 2.67 | 2.09 | 5.1 | 1.91 | 2.3 | 1.49 | 0.79 |
| Patient 25 | 4.37 | 12.36 | 13.1 | 169.19 | 99.28 | 32.04 | 11.4 | 1.24 |
| Patient 26 | 15.64 | 8.73 | 17.03 | 11.42 | 6.48 | 9.04 | 9.09 | 7.62 |
| Patient 27 | 5.21 | 36.36 | 97.24 | 19.75 | 8.96 | 1.73 | 1.2 | 3.55 |
| Patient 29 | 1.72 | 76.16 | 40.94 | 18.94 | 7.97 | 1.45 | 3.3 | 1.51 |
| Patient 30 | 1.07 | 1.48 | 2.87 | 1.82 | 1.41 | 1.18 | 1.129 | 1.05 |
| Patient 31 | 3.17 | 4.24 | 3.5 | 3.63 | 2.42 | 2.79 | 2.97 | 2.64 |
| Patient 33 | 1.04 | 2.17 | 35.45 | 10.53 | 6.87 | 1.12 | 7.69 | 7.32 |
| Patient 34 | 22.23 | 31.49 | 23.99 | 27.81 | 22.37 | 13.89 | 17.06 | 18.28 |
| Patient 35 | 6.83 | 18.8 | 77.08 | 40.2 | 14.3 | 10.51 | 9.55 | 8.79 |

P values IL-6, n = 29 (p < 0.05 = statistically significant (*)): after surgery vs. before surgery = 0.00073* (up);
Day 1 vs. before surgery = 0.000174* (up);
Day 2 vs. before surgery = 0.009* (up);
Day 3 vs. before surgery = 0.07517;
Day 7 vs. before surgery = 0.21122;
Day 14 vs. before surgery = 0.16019;
Day 28 vs. before surgery = 0.20538

Table 4 shows that HGF is significantly upregulated after surgery and at Day 1, Day 2, Day 3 and Day 7 after surgery.

TABLE 4
Significant upregulation of HGF after surgery and
at Day 1, Day 2, Day 3 and Day 7 after surgery.

| HGF | Before | After | D 1 | D 2 | D 3 | D 7 | D 14 | D 28 |
|---|---|---|---|---|---|---|---|---|
| Patient 1 | 57.08 | 60.35 | 32.26 | 65.45 | 94.61 | 53.92 | 112.74 | 94.61 |
| Patient 2 | 113.81 | 145.37 | 251.71 | 60.02 | 116.5 | 91.29 | 96.04 | 77.86 |
| Patient 3 | 175.38 | 262.14 | 277.62 | 259.95 | 363.18 | 382.47 | 288.82 | 284.32 |
| Patient 4 | 151.78 | 34.62 | 175.38 | 74.48 | 65.45 | 151.78 | 103.44 | 67.2 |
| Patient 5 | 28.9 | 18.35 | 20.07 | 39.64 | | 37.08 | 43.66 | 34.62 |
| Patient 6 | 358.23 | 379.64 | | 465.75 | 514.95 | 415.16 | 445.89 | 342.39 |
| Patient 7 | 119.22 | 266.28 | 326.75 | 319.01 | 374.26 | 454.37 | 164.51 | 148.47 |
| Patient 8 | 218.56 | 222.48 | 3557 | 841.2 | 431.85 | 363.55 | 503.26 | 226.45 |
| Patient 9 | 234.51 | 271.19 | 841.2 | 1091 | 424.05 | 201.08 | 144.16 | 118.73 |
| Patient 10 | 239.48 | 194.38 | 194.38 | 295.59 | 221.49 | 232.19 | 251.71 | 263.84 |
| Patient 11 | 15.4 | 17.54 | 21.49 | 15.4 | 19.85 | 12.81 | 15.4 | 15.4 |
| Patient 12 | 94.16 | 225.04 | 2979 | 118.73 | 349.81 | | | |
| Patient 13 | 52.07 | 69.48 | 962.84 | 629.84 | 497.46 | 43.84 | 25.95 | 36.46 |
| Patient 14 | 317.46 | 409.33 | 870.03 | 797.05 | 1132 | 814.42 | 526.17 | 296.6 |
| Patient 15 | 136.21 | 149.61 | 322.77 | 372.05 | 407.12 | 518.26 | 182 | 123.4 |
| Patient 16 | 195.79 | 94.91 | 77.06 | 83.95 | 70.48 | 68.89 | 70.48 | 55.45 |
| Patient 17 | 112.76 | 134.58 | 151.14 | 119.08 | 112.76 | 123.4 | 156.06 | 151.14 |
| Patient 18 | 146.3 | 271.62 | 415.61 | 373.62 | 392.38 | 64.23 | 146.3 | 82.2 |
| Patient 19 | 70.48 | 80.47 | 269.7 | 240.55 | 153.59 | 112.76 | 176.64 | 231.19 |
| Patient 20 | 260.63 | 203.06 | 767.9 | 243.34 | 222.64 | 193.69 | 167.19 | |
| Patient 21 | 112.76 | 256.68 | 469.44 | 347.48 | 336.76 | 382.47 | 178.67 | 112.23 |
| Patient 22 | 140.53 | 164.4 | 687.26 | 2307 | 2311 | 689.9 | 215.99 | 167.19 |
| Patient 23 | 215.99 | 262.61 | 351.8 | 209.46 | 219.3 | 190.63 | 187.6 | |
| Patient 25 | 82.2 | 193.69 | 343.18 | 1803 | 894.51 | 369.23 | 105.75 | |

P values HGF, n = 24 (p < 0.05 = statistically significant (*)): after surgery vs. before surgery = 0.0228 (up);
Day 1 vs. before surgery = 0.00724* (up);
Day 2 vs. before surgery = 0.00591* (up);
Day 3 vs. before surgery = 0.00742* (up);
Day 7 vs. before surgery = 0.00775* (up);
Day 14 vs. before surgery = 0.05204;
Day 28 vs. before surgery = 0.41475

Table 5 shows that MCP-1 is significantly upregulated after surgery and at Day 2, and Day 3 after surgery.

TABLE 5
Significant upregulation of HGF after surgery and at Day 2 and Day 3 after surgery.

| MCP-1 | Before | After | D 1 | D 2 | D 3 | D 7 | D 14 | D 28 |
|---|---|---|---|---|---|---|---|---|
| Patient 1 | 346.89 | 386.79 | 338.11 | 658.54 | 400.59 | 495.15 | 377.2 | 452.69 |
| Patient 2 | 245.05 | 278.48 | 222.31 | 326.91 | 319.9 | 263.51 | 261.16 | 223.16 |
| Patient 3 | 720.15 | 988.56 | 733.58 | 773.2 | 693.77 | 1093 | 773.36 | 660.41 |
| Patient 4 | 399.35 | 424.88 | 335.95 | 417.92 | 385.25 | 390.62 | 521.83 | 490.32 |
| Patient 5 | 791.02 | 601.99 | 384.1 | 885.88 | 1438 | 1443 | 988.78 | 1008 |
| Patient 6 | 353.11 | 848.83 | | 358.2 | 820.34 | 413.02 | 900.29 | 391.19 |
| Patient 7 | 221.63 | 281.45 | 336.88 | 426.41 | 287.8 | 292.87 | 268.97 | 256.05 |
| Patient 8 | 1247 | 1065 | 1234 | 1039 | 911.14 | 879 | 1095 | 1218 |
| Patient 9 | 579.35 | 826.58 | 548.48 | 783.86 | 477.02 | 370.25 | 346.55 | 496.7 |
| Patient 10 | 471.54 | 487.21 | 232.01 | 572.48 | 473.6 | 373.75 | 357.58 | 394.98 |
| Patient 11 | 96.18 | 38.65 | 81.3 | 107.47 | 75.94 | 76.05 | 68.32 | 71.83 |
| Patient 12 | 261.6 | 453.35 | 270.28 | 286.96 | 1688 | | | |
| Patient 13 | 397.11 | 445.47 | 713.19 | 494.76 | 374.2 | 450.79 | 406.99 | 469.9 |
| Patient 14 | 480.3 | 296.73 | 398.17 | 1105 | 737.71 | 419.47 | 370.87 | 391.7 |
| Patient 15 | 268.82 | 417.46 | 169.04 | 234.65 | 258.49 | 366.84 | 246.1 | 278.35 |
| Patient 16 | 284.55 | 381.46 | 302.93 | 328.38 | 286.67 | 313.49 | 376.19 | 371.72 |
| Patient 17 | 351.37 | 385.26 | 300.66 | 336.36 | 320.92 | 300.58 | 397.48 | 362.3 |
| Patient 18 | 801.55 | 786.31 | 781.41 | 906.76 | 969.83 | 842.96 | 856.74 | 819.62 |
| Patient 19 | 446.7 | 558.04 | 557.59 | 747.84 | 448.57 | 551.95 | 565.65 | 504.45 |
| Patient 20 | 598.71 | 761.82 | 389.14 | 572.5 | 557.08 | 406.69 | 477.34 | |
| Patient 21 | 379.56 | 530.44 | 929.07 | 452.14 | 340.1 | 835.14 | 391.5 | 379.87 |
| Patient 22 | 371.72 | 367.97 | 398.92 | 467.43 | 589.8 | 389.9 | 338.33 | 377.84 |
| Patient 23 | 425.74 | 364.69 | 780.98 | 841.53 | 406.13 | 343.35 | 304.04 | |
| Patient 25 | 142.38 | 95.91 | 148.38 | 706.81 | 423.22 | 189.78 | 180.08 | |
| Average | 445.0575 | 503.0554 | 464.2383 | 576.2913 | 570.1696 | 500.0504 | 472.6239 | 480.954 |

P values MCP-1, n = 24 (p < 0.05 = statistically significant (*)): after surgery vs. before surgery = 0.03966* (up);
Day 1 vs. before surgery = 0.35371;
Day 2 vs. before surgery = 0.00142* (up);
Day 3 vs. before surgery = 0.0423* (up);
Day 7 vs. before surgery = 0.15314;
Day 14 vs. before surgery = 0.27265;
Day 28 vs. before surgery = 0.14261

Table 6 shows that MMP-9 is significantly upregulated at after surgery and at Day 1 after surgery.

TABLE 6
Significant upregulation of MMP-9 after surgery and at Day 1 after surgery.

| MMP-9 | Before | After | D 1 | D 2 | D 3 | D 7 | D 14 | D 28 |
|---|---|---|---|---|---|---|---|---|
| Patient 1 | 30334 | 22839 | 20301 | 40973 | 14577 | 32027 | 32401 | 26151 |
| Patient 2 | 45241 | 90898 | 136613 | 30004 | 42378 | 35080 | 34161 | 26296 |
| Patient 3 | 71190 | 113651 | 111192 | 118515 | 42018 | 108063 | 64622 | 41137 |
| Patient 4 | 44246 | 98711 | 94154 | 39469 | 21053 | 59246 | 66047 | 37687 |
| Patient 5 | 131755 | 80150 | 97816 | 127227 | | 134722 | 163760 | 86542 |
| Patient 6 | 74472 | 135561 | | 74110 | 138191 | 73375 | 123812 | 78098 |
| Patient 7 | 59144 | 208787 | 100312 | 85512 | 114732 | 45942 | 65442 | 80702 |
| Patient 8 | 18591 | 33530 | 11658 | 7767 | 12198 | 34206 | 104099 | 32356 |
| Patient 9 | 16188 | 70639 | 15865 | 67304 | 14463 | 41384 | 31427 | 16144 |
| Patient 10 | 43867 | 81341 | 79670 | 32528 | 40120 | 44362 | 23861 | 24900 |
| Patient 11 | 15912 | 70977 | 48904 | 26991 | 37247 | 24229 | 19088 | 25812 |
| Patient 12 | 85221 | 260288 | 30219 | 17246 | 83729 | | | |
| Patient 13 | 92784 | 242844 | 161006 | 45267 | 51178 | 4864 | 46121 | 83203 |
| Patient 14 | 87265 | 106362 | 47663 | 109945 | 85300 | 122075 | 97364 | 48542 |
| Patient 15 | 47770 | 61415 | 48046 | 31258 | 38451 | 50022 | 33181 | 20721 |
| Patient 16 | 40533 | 69646 | 26255 | 20285 | 20804 | 31413 | 25980 | 15726 |
| Patient 17 | 92684 | 144133 | 122538 | 74234 | 75484 | 76795 | 164105 | 110434 |
| Patient 18 | 101130 | 146579 | 29451 | 38215 | 77079 | 121520 | 76035 | 58381 |
| Patient 19 | 13268 | 23670 | 20954 | 12659 | 19223 | 16815 | 16891 | 14275 |
| Patient 20 | 104610 | 117682 | | 59977 | 67947 | 87903 | 66984 | |
| Patient 21 | 46298 | 181529 | 60010 | 29982 | 54875 | 121762 | 62572 | 48526 |
| Patient 22 | 30655 | 91081 | 35622 | 22039 | 60140 | 38225 | 37163 | 44286 |
| Patient 23 | 91006 | 158650 | 179892 | 64994 | 62357 | 55323 | 78385 | |
| Patient 25 | 37102 | 147097 | 78888 | 50225 | 103001 | 47110 | 54059 | |
| Average | 59219.42 | 114919.2 | 70774.05 | 51113.58 | 55501.96 | 64194.04 | 64676.52 | 45995.95 |

P values MMP-9, n = 24 (p < 0.05 = statistically significant (*)): after surgery vs. before surgery = 2.03767E−05* (up);
Day 1 vs. before surgery = 0.066421* (up);
Day 2 vs. before surgery = 0.09712;
Day 3 vs. before surgery = 0.46457;
Day 7 vs. before surgery = 0.10848;
Day 14 vs. before surgery = 0.16279;
Day 28 vs. before surgery = 0.03068* (down)

Table 7 shows that PGF levels are upregulated after surgery and at Day 1, Day 2, Day 3, Day 7, Day 14 and Day 28 after surgery.

TABLE 7
Significant upregulation of of PGF levels at Day 1 after surgery and
at Day 1, Day 2, Day 3, Day 7, Day 14 and Day 28 after surgery.

| PGF | Before | After | D 1 | D 2 | D 3 | D 7 | D 14 | D 28 |
|---|---|---|---|---|---|---|---|---|
| Patient 1 | 2.82 | 2.91 | 2.01 | 2.23 | 4.09 | 4.69 | 4.42 | 3.31 |
| Patient 2 | 4.31 | 5.35 | 1.23 | 3.54 | 6.35 | 4.78 | 4.15 | 4.62 |
| Patient 3 | 1.15 | 1.28 | 2.91 | 2.27 | 2.62 | 2.86 | 1.95 | 4.69 |
| Patient 4 | 2.38 | 0.61 | 2.62 | 2.16 | 1.65 | 5.22 | 3.08 | 2.16 |
| Patient 5 | 1.44 | 1.56 | 2.91 | 5.52 | | 3.22 | 3.54 | 1.62 |
| Patient 6 | 12.28 | 12.74 | | 13.94 | 15.7 | 14.03 | 16.92 | 11.73 |
| Patient 7 | 6.09 | 3.39 | 9.03 | 7.63 | 3.54 | 4.94 | 4.86 | 9.66 |
| Patient 8 | 9.12 | 14.03 | 16.82 | 11.55 | 13.38 | 22.56 | 18.79 | 15.7 |
| Patient 9 | 11.73 | 11.92 | 16.08 | 12.19 | 8.65 | 11.17 | 7.65 | 6.44 |
| Patient 10 | 2.54 | 1.39 | 0.73 | 2.86 | 3.74 | 2.19 | 7.46 | 6.94 |
| Patient 11 | 0.72 | 0.72 | 0.99 | 0.91 | 1.11 | 1.08 | 1.02 | 0.86 |
| Patient 12 | 3.65 | 6.44 | 3.99 | 16.39 | 69.47 | | | |
| Patient 13 | 13.75 | 13.26 | 18.55 | 14 | 13.02 | 12.94 | 6.35 | 9.98 |
| Patient 14 | 2.7 | 3.46 | 6.35 | 18.92 | 12.7 | 6.83 | 5.71 | 7.03 |
| Patient 15 | 4.96 | 7.76 | 18.64 | 12.86 | 10.31 | 17.52 | 11.53 | 16.18 |
| Patient 16 | 14.65 | 6.52 | 15.79 | 7.83 | 9.62 | 10.43 | 9.4 | 13.68 |
| Patient 17 | 4 | 3.38 | 6.35 | 4.07 | 5.92 | 4.61 | 2.94 | 5.13 |
| Patient 18 | 9.18 | 6.52 | 13.9 | 12.95 | 4.61 | 13.17 | 13.46 | 14.57 |
| Patient 19 | 5.44 | 3.16 | 14.35 | 10.91 | 12.51 | 10.43 | 9.4 | 12.66 |
| Patient 20 | 7.56 | 8.89 | 9.86 | 10.88 | 8.53 | 8.71 | 11.09 | |
| Patient 21 | 0.76 | 0.72 | 2.22 | 1.95 | 1.35 | 1.95 | 0.85 | 0.85 |
| Patient 22 | 6.36 | 4.38 | 15.35 | 16.69 | 19.06 | 18.73 | 11.52 | 11.09 |
| Patient 23 | 8.99 | 6.21 | 3.53 | 9.86 | 8.26 | 7.48 | 10.26 | |
| Patient 25 | 8.44 | 15.89 | 5.91 | 28.7 | 11.96 | 16.69 | 16.13 | |
| Average | 6.0425 | 5.937083 | 8.266087 | 9.617083 | 10.78913 | 8.966522 | 7.933913 | 7.945 |

P values PGF, n = 24 (p < 0.05 = statistically significant (*)): after surgery vs. before surgery = 0.43143;
Day 1 vs. before surgery = 0.00596* (up);
Day 2 vs. before surgery = 0.00394* (up);
Day 3 vs. before surgery = 0.06693;
Day 7 vs. before surgery = 0.00423* (up);
Day 14 vs. before surgery = 0.02303* (up);
Day 28 vs. before surgery = 0.012592* (up)

Table 8 shows that TGF-beta is significantly down-regulated at Day 1 after surgery.

TABLE 8
Significant down-regulation of TGF-beta at Day 1 after surgery.

| TGF-B | Before | After | D 1 | D 2 | D 3 | D 7 | D 14 | D 28 |
|---|---|---|---|---|---|---|---|---|
| Patient 1 | 3744 | 6471 | 3612 | 4602 | 4448 | 5411 | 4696 | 3793 |
| Patient 2 | 4101 | 9574 | 2555 | 6351 | 5191 | 4031 | 3461 | 3391 |
| Patient 3 | 5110 | 20387 | 13771 | 11451 | 6777 | 24502 | 14167 | 11457 |
| Patient 4 | 7370 | 8144 | 7014 | 8676 | 9775 | 6433 | 4626 | 4415 |
| Patient 5 | 8656 | 7232 | 4415 | 7814 | No spec | 15497 | 13421 | 8592 |
| Patient 6 | 13630 | 25299 | | 9438 | 15326 | 12677 | 20029 | 16594 |
| Patient 7 | 6028 | 4890 | 1401 | 1782 | 2089 | 2871 | 22252 | 9313 |
| Patient 8 | 2089 | 2953 | 1341 | 1627 | 2239 | 4303 | 8684 | 4281 |
| Patient 9 | 5164 | 5472 | 1807 | 7755 | 2027 | 4575 | 4082 | 1670 |
| Patient 10 | 3410 | 4735 | 4818 | 6188 | 7141 | | 5587 | 2239* |
| Patient 11 | 3678 | 4287 | 6232 | 4033 | 3281 | 2393 | 2675 | 3239 |
| Patient 12 | 4209 | 6096 | 3270 | 2027 | 2531 | | | |
| Patient 13 | 2139 | 2365 | 1783 | 1550 | 2738 | 5725 | 2701 | 5697 |
| Patient 14 | 5078 | 6147 | 1670 | 15923 | 4511 | 5500 | 8718 | 6552 |
| Patient 15 | 6547 | 3873 | 3078 | 1424 | 1997 | 4547 | 6372 | 4691 |
| Patient 16 | 5482 | 9694 | 3545 | 3667 | 4008 | 7427 | 4547 | 3730 |
| Patient 17 | 2029 | 2563 | 2214 | 2273 | 2764 | 1739 | 2728 | 2108 |
| Patient 18 | 7556 | 8084 | 1793 | 2789 | 6253 | 22060 | 17609 | 9217 |
| Patient 19 | 9402 | 11680 | 3793 | 12276 | 6337 | 8348 | 11030 | 12377 |
| Patient 20 | 5156 | 11213 | 8202 | 7606 | 10103 | 8658 | 7945 | 6196 |
| Patient 21 | 15937 | 6275 | 3493 | 2695 | 6226 | 21404 | 13383 | 8973 |
| Patient 22 | 9433 | 7789 | 5790 | 1949 | 3186 | 3146 | 5437 | 8488 |
| Patient 23 | 14817 | 10878 | 8056 | 8475 | 8938 | 5666 | 4562 | 5670 |
| Patient 25 | 4298 | 2441 | 1495 | 3156 | 6160 | 9128 | 5100 | 4209 |
| Patient 26 | 4512 | 6241 | 3320 | 5041 | 5546 | 5951 | 6266 | 7050 |
| Patient 27 | 3951 | 3236 | 2206 | 4904 | 4160 | 8310 | 3937 | 5593 |
| Patient 29 | 1508 | 3009 | 1828 | 716.1 | 566.59 | 1835 | 1610 | 2065 |
| Patient 30 | 10961 | 9165 | 5861 | 8274 | 8779 | 6180 | 3800 | 6176 |
| Patient 31 | 4552 | 12159 | 5721 | 5985 | 5106 | 4649 | 3483 | 7481 |
| Patient 33 | 12832 | 8633 | 2448 | 9966 | 11200 | 10371 | 11339 | 8215 |
| Patient 34 | 4785 | 2894 | 4356 | 2994 | 3571 | 8839 | 10484 | 6394 |
| Patient 35 | 3588 | 2756 | 714.53 | 1296 | 3378 | 1520 | 1995 | 1366 |
| | 6304.75 | 7394.844 | 3922.662 | 5459.566 | 5374.922 | 7789.867 | 7636.323 | 6299.767 |

P values TGF-beta, n = 32 (p < 0.05 = statistically significant (*)): after surgery vs. before surgery = 0.0942;
Day 1 vs. before surgery = 0.00251* (down);
Day 2 vs. before surgery = 0.013586;
Day 3 vs. before surgery = 0.06321;
Day 7 vs. before surgery = 0.10053;
Day 14 vs. before surgery = 0.08699;
Day 28 vs. before surgery = 0.39017

Table 9 shows that IL-8 is unchanged after surgery.

TABLE 9
IL-8 levels are unchanged after surgery.

| IL8 | Before | After | D 1 | D 2 | D 3 | D 7 | D 14 | D 28 |
|---|---|---|---|---|---|---|---|---|
| Patient 1 | 5.7 | 5.7 | 12.24 | 13.1 | 5.98 | 11.94 | 4.08 | 5.7 |
| Patient 2 | 4.61 | 18.78 | 24.58 | 19.02 | 20.97 | 21.12 | 22.45 | 14.29 |
| Patient 3 | 55.13 | 55.24 | 58.31 | 49.09 | 50.26 | 62.19 | 47.85 | 37.81 |
| Patient 4 | 13.38 | 11.13 | 7.03 | 7.1 | 5.66 | 8.86 | 7.12 | 6.93 |
| Patient 6 | 10.98 | 25.98 | | 9.69 | 17.26 | 13.84 | 14.79 | 9.02 |
| Patient 7 | 0.99 | 1.29 | 3.55 | 3.59 | 2.33 | 9.47 | 3.43 | 1.96 |
| Patient 8 | 9.67 | 8.31 | 17.69 | 5.96 | 4.35 | 6.1 | 10.54 | 7.16 |
| Patient 9 | 45.86 | 41.6 | 36.66 | 27.53 | 29.83 | 26.38 | 32.74 | 37.83 |
| Patient 10 | 4.66 | 5.15 | 3.83 | 4.8 | 5.34 | 3.68 | 4.37 | 4.48 |
| Patient 11 | 8.99 | 22.76 | 20.28 | 24.79 | 24.39 | 31.45 | 29.72 | 31.03 |
| Patient 13 | 10.25 | 9.5 | 18.55 | 8.17 | 3.92 | 7.04 | 4.32 | 6.94 |
| Patient 14 | 9.33 | 6.24 | 12.53 | 25.23 | 36.91 | 13.85 | 9.7 | 6.01 |
| Patient 15 | 1.64 | 5.45 | 3.59 | 2.23 | 3.1 | 4.76 | 2.67 | 2.52 |
| Patient 16 | 2.49 | 3.23 | 5.09 | 2.52 | 2.23 | 3.23 | 3.69 | 2.7 |
| Patient 17 | 34.55 | 31.04 | 6 | 13.21 | 10.87 | 42.33 | 33.9 | 13.46 |
| Patient 18 | 122.19 | 85.25 | 87.33 | 108.98 | 92.75 | 92.09 | 90.12 | 94.39 |
| Patient 19 | 36.22 | 40.56 | 24.75 | 17.24 | 17.19 | 14.11 | 12.48 | 18.6 |
| Patient 20 | 3.94 | 9.34 | 4.39 | 5.55 | 5.46 | 4.53 | 4.26 | 2.66 |
| Patient 21 | 13.58 | 20.4 | 22.89 | 11.42 | 13.39 | 27.69 | 17.01 | 17.79 |
| Patient 22 | 16.49 | 10.79 | 21.12 | 28.42 | 30.35 | 18.53 | 11.47 | 10.43 |
| Patient 23 | 5.42 | 3.7 | 51.06 | 9.75 | 3 | 3.15 | 2.65 | 2.69 |
| Patient 25 | 4.82 | 5.07 | 6.33 | 18.92 | 22.22 | 19.93 | 7.59 | 3.25 |
| Patient 26 | 10.42 | 8.42 | 9.49 | 7.79 | 6.88 | 10.07 | 7.83 | 8.48 |
| Patient 27 | 6.57 | 8.24 | 16.61 | 5.89 | 5.39 | 5.43 | 3.8 | 3.26 |
| Patient 29 | 3 | 5.08 | 6.53 | 4.5 | 3.75 | 3.6 | 8.39 | 5.03 |
| Patient 30 | 7.38 | 6.84 | 7.2 | 6.23 | 7.03 | 6.95 | 5.98 | 6.16 |
| Patient 31 | 23.02 | 19.89 | 21.63 | 21.38 | 19.86 | 19.78 | 20.07 | 20.01 |
| Patient 33 | 5.2 | 4.95 | 14.2 | 5.42 | 5.83 | 6.46 | 6.41 | 7.17 |
| Patient 34 | 10.8 | 8.43 | 8.23 | 12.39 | 10.88 | 7.25 | 8.3 | 9.83 |
| Patient 35 | 8.05 | 7.93 | 20.06 | 10.51 | 6.05 | 7.77 | 8.7 | 8.19 |

P values IL-8, n = 32 (p < 0.05 = statistically significant (*)): after surgery vs. before surgery = 0.49204;
Day 1 vs. before surgery = 0.18784;
Day 2 vs. before surgery = 0.46249;
Day 3 vs. before surgery = 0.36836;
Day 7 vs. before surgery = 0.37866;
Day 14 vs. before surgery = 0.1813;
Day 28 vs. before surgery = 0.04077

Table 10 shows that TNF-alpha levels are down-regulated at Day 1 and Day 2 after surgery.

TABLE 10
Significant down-regulation of TNF-alpha levels at Day 1 and Day 2 after surgery.

| TNF-A | Before | After | D1 | D2 | D3 | D7 | D14 | D28 |
|---|---|---|---|---|---|---|---|---|
| Patient 1 | 8.63 | 9.31 | 7.72 | 10.46 | 8.39 | 8.93 | 7.03 | 8.9 |
| Patient 2 | 8.67 | 12.1 | 7.77 | 12.55 | 12.28 | 13.14 | 12.78 | 8.16 |
| Patient 3 | 28.48 | 40.81 | 34.02 | 32 | 26.54 | 50.3 | 34.51 | 27.23 |
| Patient 4 | 11.56 | 13.15 | 11.34 | 12.6 | 11.74 | 10.63 | 11.02 | 11.09 |
| Patient 5 | 125.27 | 101.39 | 24.97 | 92.13 | 108.44 | 83.58 | 107.55 | 95.79 |
| Patient 6 | 20.58 | 23.38 | | 17.98 | 20.97 | 22.35 | 26.41 | 21.32 |
| Patient 7 | 9.23 | 8.16 | 10.7 | 12.46 | 8.87 | 24.77 | 14.5 | 13.1 |
| Patient 8 | 17.46 | 12.82 | 12.55 | 11.57 | 9.56 | 10.87 | 15.36 | 10.78 |
| Patient 9 | 58.32 | 56.36 | 44.68 | 55.83 | 91.65 | 46.55 | 32.1 | 32.21 |
| Patient 10 | 14.68 | 13.08 | 8.02 | 12.57 | 16.66 | 10.46 | 19 | 17.84 |
| Patient 11 | 9.28 | 2.36 | 2.17 | 2.2 | 2.25 | 2.12 | 2.11 | 2.69 |
| Patient 12 | 12.94 | 11.32 | 6.47 | 5.45 | 11.47 | | | |
| Patient 13 | 11.78 | 13.95 | 14.49 | 13.42 | 9.16 | 14.1 | 10.27 | 11.3 |
| Patient 14 | 17.65 | 10.96 | 11.35 | 15.53 | 16.52 | 15.68 | 11.61 | 13.32 |
| Patient 15 | 11.23 | 10.77 | 7.15 | 6.43 | 7.47 | 13.96 | 11.54 | 12.49 |
| Patient 16 | 10.59 | 14.98 | 11.72 | 9.21 | 9.21 | 10.95 | 13.77 | 13.14 |
| Patient 17 | 7.68 | 12.21 | 6.33 | 14.02 | 6.43 | 8.71 | 9.24 | 11 |
| Patient 18 | 69.95 | 39.7 | 40.93 | 54.76 | 43.44 | 44.23 | 37.57 | 46.81 |
| Patient 19 | 19.34 | 17.54 | 15.97 | 18.87 | 15.51 | 19.18 | 27.93 | 24.55 |
| Patient 20 | 16.39 | 20.12 | 11.4 | 15.94 | 18.68 | 15.44 | 16.1 | |
| Patient 21 | 32.7 | 21.82 | 20.87 | 13.32 | 16.65 | 36.62 | 27.02 | 21.28 |
| Patient 22 | 13.44 | 9.21 | 12.5 | 12.33 | 17.81 | 20.85 | 15.01 | 13.86 |
| Patient 23 | 18.07 | 13.89 | 24.37 | 20.71 | 12.7 | 9.08 | 8.97 | |
| Patient 25 | 10.64 | 7.56 | 7.64 | 19.35 | 26.19 | 26.94 | 2.72 | |
| Average | 23.52333 | 20.70625 | 15.44043 | 20.48708 | 22.02458 | 22.58435 | 20.61391 | 20.843 |

P values TNF-A, n = 24 (p < 0.05 = statistically significant (*)):
after surgery vs. before surgery = 0.06717;
Day 1 vs. before surgery = 0.0393* (down);
Day 2 vs. before surgery = 0.05497* (down);
Day 3 vs. before surgery = 0.2551;
Day 7 vs. before surgery = 0.30798;
Day 14 vs. before surgery = 0.63612;
Day 28 vs. before surgery = 0.03266* (Down)

Table 11 shows that FGF-2 levels are down-regulated at Day 1 after surgery.

TABLE 11
Significant down-regulation of FGF-2 levels at Day 1 after surgery.

| FGF2 | Before | After | D1 | D2 | D3 | D7 | D14 | D28 |
|---|---|---|---|---|---|---|---|---|
| Patient 1 | 42.93 | 44.87 | 16 | 35.81 | 29.65 | 133.74 | 8781 | 8781 |
| Patient 2 | 16 | 92.5 | 61.43 | 119.03 | 112.22 | 84.41 | 108.75 | 25.21 |
| Patient 3 | 97.72 | 203.36 | 164.84 | 147.7 | 118.06 | 214.25 | 162.61 | 140.79 |
| Patient 4 | 125.28 | 120.96 | 121.44 | 113.68 | 104.78 | 107.77 | 87.93 | 108.76 |
| Patient 6 | 53.53 | 105.23 | | 26.61 | 56.07 | 70.23 | 94.83 | 90.16 |
| Patient 9 | 240.2 | 224.58 | 181.76 | 197.03 | 317.19 | 235.33 | 280.08 | 320.2 |
| Patient 10 | 121.92 | 111.23 | 114.66 | 110.24 | 129.08 | 184.14 | 112.22 | 99.86 |
| Patient 12 | 348.27 | 249.8 | 274.14 | 168.27 | 233.26 | | | |
| Patient 13 | 96.74 | 81.11 | 99.03 | 46.16 | 48.37 | 95.2 | 30.66 | 111.81 |
| Patient 14 | 26.84 | 23.81 | 34.79 | 30.09 | 22.38 | 29.54 | 31.23 | 42.61 |
| Patient 17 | 99.44 | 104.76 | 3.2 | 90.93 | 29.51 | 113.54 | 102.44 | 83.93 |
| Patient 18 | 412.7 | 223.88 | 162.95 | 173.66 | 135.66 | 225.68 | 241.13 | 297.43 |
| Patient 19 | 288.74 | 298.6 | 208.56 | 197.69 | 204.08 | 177.83 | 536.84 | 437.5 |
| Patient 20 | 99.28 | 142.05 | 106.71 | 111.92 | 134.32 | 113.3 | 108.11 | |
| Patient 21 | 234.08 | 265.32 | 160.8 | 149.64 | 222.22 | 253.51 | 306.24 | 371.18 |
| Patient 22 | 162.63 | 128.43 | 146.81 | 174.1 | 160.18 | 152.76 | 129.09 | 112.27 |
| Patient 23 | 134.32 | 94.22 | 81.15 | 145.54 | 82.68 | 23.45 | 1.04 | |
| Patient 25 | 61.5 | 0.1 | 1.38 | 89.81 | 118.41 | 175.89 | 93.12 | |
| Average | 147.8956 | 139.7117 | 114.0971 | 118.2172 | 125.4511 | 140.6218 | 659.2541 | 787.3364 |

P values FGF-2, n = 18 (p < 0.05 = statistically significant (*)):
after surgery vs. before surgery = 0.30268;
Day 1 vs. before surgery = 0.01792* (down);
Day 2 vs. before surgery = 0.06712;
Day 3 vs. before surgery = 0.13529;
Day 7 vs. before surgery = 0.41027;
Day 14 vs. before surgery = 0.16193;
Day 28 vs. before surgery = 0.16036

Table 12 shows that HIGF-2 levels are down-regulated after surgery and at Day 1, Day 2, Day 3 and Day 7 after surgery.

TABLE 12
Significant down-regulation of HIGF-2 levels after surgery and at Day 1, Day 2,
Day 3 and Day 7 after surgery.

| HIGF-1 | Before | After | D1 | D2 | D3 | D7 | D14 | D28 |
|---|---|---|---|---|---|---|---|---|
| Patient 1 | 53294 | 58681 | 58718 | 68676 | 58142 | 62773 | 64936 | 58288 |
| Patient 2 | 112879 | 91096 | 77738 | 90811 | 101658 | 93662 | 88862 | 102592 |
| Patient 3 | 51926 | 41721 | 49497 | 42847 | 42878 | 53021 | 59487 | 64706 |
| Patient 4 | 143091 | 122231 | 140389 | 104843 | 159738 | 170463 | 153240 | 155396 |
| Patient 5 | 63645 | 48071 | 63327 | 55119 | | 57425 | 73422 | 70598 |
| Patient 6 | 73406 | 58298 | | 56529 | 63441 | 38476 | 63037 | 3030 |
| Patient 7 | 58062 | 61038 | 60599 | 33311 | 25206 | 27815 | 46976 | 56953 |
| Patient 8 | 74270 | 59921 | 23449 | 26649 | 27641 | 31790 | 30693 | 29683 |
| Patient 9 | 58858 | 35829 | 15589 | 10822 | 11832 | 20204 | 47494 | 73628 |
| Patient 10 | 103435 | 70020 | 69798 | 86670 | 77946 | 108702 | 97357 | 99872 |
| Patient 11 | 122382 | 106851 | 97464 | 120096 | 117840 | 117126 | 118628 | 134841 |
| Patient 12 | 115826 | 92529 | 90642 | 63170 | 40030 | | | |
| Patient 13 | 99507 | 111482 | 140174 | 125796 | 83699 | 109025 | 120774 | 110408 |
| Patient 14 | 98598 | 94955 | 70766 | 82905 | 61825 | 69113 | 85033 | 90383 |
| Patient 15 | 68539 | 54604 | 55716 | 52765 | 46212 | 44856 | 81733 | 89796 |
| Patient 16 | 103557 | 96796 | 97945 | 103380 | 96542 | 98095 | 115355 | 92511 |
| Patient 17 | 118427 | 105697 | 118427 | 113604 | 117461 | 112712 | 103667 | 114927 |
| Patient 18 | 33743 | 74384 | 53443 | 44078 | 18813 | 20312 | 46716 | 90709 |
| Patient 19 | 83251 | 74312 | 71643 | 62297 | 47686 | 24409 | 60467 | 84805 |
| Patient 20 | 77199 | 71117 | 78724 | 84735 | 84321 | 75913 | 76489 | |
| Patient 21 | 90831 | 81207 | 78384 | 65364 | 64236 | 81207 | 96657 | 82076 |
| Patient 22 | 99758 | 88512 | 93532 | 65622 | 52604 | 45205 | 110191 | 80591 |
| Patient 23 | 69832 | 75968 | 78516 | 85844 | 88553 | 69372 | 74399 | |
| Average | 85836.83 | 77187.83 | 76567.27 | 71562.3 | 67650.18 | 69621.64 | 82540.14 | 84289.65 |

P values HIGF-1, n = 23 (p < 0.05 = statistically significant (*)):
after surgery vs. before surgery = 0.00598* (down);
Day 1 vs. before surgery = 0.02042* (down);
Day 2 vs. before surgery = 0.00206* (down);
Day 3 vs. before surgery = 0.00044* (down);
Day 7 vs. before surgery = 0.00234* (down);
Day 14 vs. before surgery = 0.28478;
Day 28 vs. before surgery = 0.41181

Table 13 shows that IL1B levels are down-regulated at Day 1 and Day 2 after surgery.

TABLE 13
Significant down-regulation of IL1B levels after surgery and at Day 1 and Day 2
after surgery.

| IL1B | Before | After | D1 | D2 | D3 | D7 | D14 | D28 |
|---|---|---|---|---|---|---|---|---|
| Patient 1 | 1.13 | 1.2 | 0.56 | 0.82 | 0.7 | 0.52 | 0.5 | 0.55 |
| Patient 2 | 0.68 | 1.57 | 0.51 | 0.82 | 1 | 0.61 | 0.73 | 0.45 |
| Patient 3 | 5.26 | 16.37 | 9.04 | 11.36 | 6.35 | 19.45 | 12.38 | 14.42 |
| Patient 4 | 1.63 | 2.14 | 1.89 | 2.46 | 1.89 | 1.3 | 1.46 | 1.61 |
| Patient 5 | 42.51 | 16.51 | 7.28 | 22.67 | 43.72 | 45.83 | 46.83 | 86.37 |
| Patient 6 | 1.32 | 3.84 | | 0.98 | 1.12 | 1.22 | 2.41 | 1.69 |
| Patient 7 | 0.42 | 0.42 | 0.27 | 0.31 | 0.33 | 0.3 | 1.16 | 0.58 |
| Patient 8 | 2.71 | 1.34 | 0.5 | 0.35 | 0.27 | 0.76 | 2.33 | 0.66 |
| Patient 9 | 11.27 | 8.13 | 2.05 | 6.88 | 55.33 | 13.46 | 3.58 | 1.62 |
| Patient 10 | 1.92 | 1.37 | 1.79 | 1.66 | 1.84 | 2 | 1.04 | 0.93 |
| Patient 11 | 5.17 | 0.35 | 0.39 | 0.29 | 0.24 | 0.23 | 0.23 | 0.36 |
| Patient 12 | 0.89 | 1.26 | 0.38 | 0.27 | 0.7 | | | |
| Patient 13 | 1.24 | 1.27 | 1.78 | 0.93 | 0.97 | 1.81 | 0.85 | 1.4 |
| Patient 14 | 0.42 | 0.35 | 0.31 | 0.57 | 0.46 | 0.39 | 0.49 | 0.41 |
| Patient 15 | 0.49 | 0.35 | 0.21 | 0.17 | 0.2 | 0.34 | 0.34 | 0.28 |
| Patient 16 | 0.98 | 2.21 | 0.6 | 0.6 | 0.67 | 1.18 | 0.88 | 0.57 |
| Patient 17 | 3.94 | 9.52 | 2.42 | 3.14 | 2.35 | 3.66 | 4.03 | 5.56 |
| Patient 18 | 4.9 | 3.4 | 0.54 | 1.49 | 0.91 | 8.4 | 2.77 | 2.82 |
| Patient 19 | 17.84 | 20.93 | 17.91 | 14.18 | 16 | 6.26 | 80.23 | 51.31 |
| Patient 20 | 1.47 | 4.19 | 2.08 | 2.71 | 4.49 | 2.87 | 2.43 | 1.08 |
| Patient 21 | 10.62 | 3.2 | 1.42 | 1.08 | 3.79 | 16 | 7.37 | 4.34 |
| Patient 22 | 3.12 | 2.13 | 2.47 | 1.44 | 1.37 | 2.92 | 1.78 | 1.73 |
| Patient 23 | 4.58 | 2.17 | 1.65 | 6.43 | 1.6 | 0.91 | 0.54 | 0.63 |
| Patient 25 | 1.98 | 1.27 | 0.99 | 2 | 5.32 | 7.81 | 1.92 | 0.78 |
| Patient 26 | 1.55 | 1.16 | 1.23 | 0.86 | 1.26 | 1.21 | 1.44 | 1.06 |
| Patient 27 | 0.65 | 0.7 | 0.9 | 0.83 | 0.92 | 1.71 | 0.86 | 0.62 |
| Patient 29 | 0.77 | 0.65 | 0.69 | 0.57 | 0.59 | 0.6 | 0.65 | 0.73 |
| Patient 30 | 0.64 | 0.74 | 0.69 | 0.69 | 0.7 | 0.58 | 0.78 | 0.74 |
| Patient 31 | 1.09 | 0.82 | 0.96 | 0.94 | 0.92 | 0.88 | 1.02 | 0.96 |
| Patient 33 | 0.38 | 0.46 | 3.55 | 0.36 | 0.36 | 0.4 | 0.33 | 0.36 |
| Patient 34 | 0.66 | 0.66 | 0.67 | 0.74 | 0.78 | 0.52 | 0.48 | 0.61 |
| Patient 35 | 0.71 | 0.62 | 2.57 | 1.3 | 1.48 | 0.7 | 0.75 | 0.6 |
| Average | 4.154375 | 3.478125 | 2.203226 | 2.809375 | 4.957188 | 4.671935 | 5.89 | 5.994516 |

P values IL1B, n = 32 (p < 0.05 = statistically significant (*)):
after surgery vs. before surgery = 0.24645;
Day 1 vs. before surgery = 0.0514* (down);
Day 2 vs. before surgery = 0.04013* (down);
Day 3 vs. before surgery = 0.29054;
Day 7 vs. before surgery = 0.28257;
Day 14 vs. before surgery = 0.219;
Day 28 vs. before surgery = 0.1794

Table 14 shows that IL1A levels are down-regulated after surgery and at Day 1, Day 2 and Day 3 after surgery.

TABLE 14
Significant down-regulation of IL1A levels after surgery and at Day 1, Day 2 and
Day 3 after surgery.

| IL1A | Before | After | D1 | D2 | D3 | D7 | D14 | D28 |
|---|---|---|---|---|---|---|---|---|
| Patient 1 | 0.07 | 0.02 | 0.01 | 0.02 | 0.02 | 0.01 | 0.02 | 0.02 |
| Patient 2 | 0.51 | 21 | 0.28 | 52.72 | 31.87 | 62.61 | 24.94 | 0.38 |
| Patient 3 | 93.67 | 86.91 | 93.94 | 92.58 | 73.51 | 131.75 | 96.96 | 105 |
| Patient 4 | 2.59 | 3.93 | 1.44 | 4.43 | 259 | 0.47 | 1.59 | 1.03 |
| Patient 5 | 839.78 | 406.14 | 90.41 | 671.2 | 606.66 | 779.4 | 561.14 | 518.17 |
| Patient 8 | 449.83 | 301.68 | 60.53 | 20.55 | 8.6 | 145.86 | 37.24 | 0.51 |
| Patient 9 | 568.39 | 379.06 | 249.88 | 321.98 | 518.49 | 760.94 | 1171 | 1471 |
| Patient 10 | 1.51 | 1.9 | 1.44 | 1.9 | 1.9 | 1.44 | 3.79 | 0.38 |
| Patient 13 | 88.51 | 38.7 | 82.98 | 34.55 | 25.24 | 44.04 | 37.01 | 45.91 |
| Patient 16 | 0.64 | 29.65 | 0.07 | 0.02 | 0.06 | 3.99 | 14.42 | 0.05 |
| Patient 17 | 126.15 | 160.83 | 0.64 | 24.13 | 0.5 | 130.46 | 125.82 | 48.73 |
| Patient 18 | 645.14 | 202.53 | 82.08 | 127.47 | 100.42 | 160.13 | 204.74 | 328.84 |
| Patient 19 | 330.49 | 315.26 | 246.36 | 237.89 | 265.07 | 166.09 | 622.71 | 577.38 |
| Patient 20 | 0.02 | 3.41 | 0.04 | 0.13 | 0.89 | 0.13 | 0.12 | |
| Patient 21 | 64.92 | 6.33 | 0.21 | 0.05 | 5.96 | 108.8 | 35.39 | 18.3 |
| Patient 22 | 56.81 | 23.6 | 33.03 | 75.73 | 42.65 | 38.38 | 24.33 | 26.62 |
| Patient 25 | 15.13 | 3 | 6.33 | 27.82 | 74.9 | 138.43 | 29.06 | 0.24 |
| Patient 26 | 123.1 | 113.3 | 110.9 | 97.93 | 89.58 | 94.87 | 97.93 | 106.12 |
| Patient 30 | 0.17 | 0.19 | 0.3 | 0.08 | 0.19 | 0.27 | 0.17 | 0.04 |
| Patient 31 | 33.06 | 17.93 | 31.04 | 25.38 | 18.93 | 23.25 | 22.5 | 22.68 |
| Patient 34 | 108.53 | 78.91 | 90.9 | 89.27 | 95 | 58.83 | 86.59 | 84.46 |
| Patient 35 | 308.11 | 110.84 | 275.49 | 110.27 | 41.04 | 128.11 | 134.87 | 265.68 |
| Average | 175.3241 | 104.7782 | 66.28636 | 91.64091 | 91.09409 | 135.3755 | 151.47 | 172.4543 |

P values IL1A, n = 22 (p < 0.05 = statistically significant (*)):
after surgery vs. before surgery = 0.01151* (down);
Day 1 vs. before surgery = 0.01105* (down);
Day 2 vs. before surgery = 0.00731* (down);
Day 3 vs. before surgery = 0.009* (down);
Day 7 vs. before surgery = 0.09992;
Day 14 vs. before surgery = 0.29876;
Day 28 vs. before surgery = 0.42089

Table 15 shows that VEGF levels are down-regulated after surgery and at Day 1, Day 2, Day 3, Day 7, Day 14 and Day 28 after surgery,

TABLE 15
Significant down-regulation of VEGF levels after surgery and at Day 1, Day 2,
Day 3, Day 7, Day 14 and Day 28 after surgery.

| VEGF | Before | After | D1 | D2 | D3 | D7 | D14 | D28 |
|---|---|---|---|---|---|---|---|---|
| Patient 1 | 1.38 | 7.24 | 0.23 | 0.37 | 0.23 | 0 | 0.06 | 0 |
| Patient 2 | 102.69 | 605.8 | 693.67 | 736.56 | 738.69 | 732.29 | 801.82 | 599.13 |
| Patient 3 | 1725 | 1392 | 1458 | 1384 | 1430 | 1469 | 1405 | 1411 |
| Patient 4 | 500.01 | 435.52 | 374.27 | 376.4 | 353.17 | 409.09 | 303.88 | 409.09 |
| Patient 5 | 14476 | 11045 | 6037 | 11008 | 11017 | 11595 | 13519 | 8897 |
| Patient 6 | 123.22 | 235.09 | | 98.88 | 136.2 | 123.22 | 205.24 | 153.23 |
| Patient 7 | 24.38 | 228.21 | 981.48 | 143.22 | 981.48 | 58.06 | 110.35 | 47.64 |
| Patient 8 | 186.43 | 187.76 | 86.25 | 47.64 | 29.76 | 90.03 | 155.87 | 47.64 |
| Patient 9 | 281.84 | 259.75 | 178.41 | 332.06 | 714.79 | 514.9 | 833.28 | 1293 |
| Patient 10 | 470.62 | 411.79 | 422.46 | 424.22 | 479.5 | 533.35 | 427.89 | 426.37 |
| Patient 12 | 581.94 | 358.87 | 239.19 | 151.84 | 202 | | | |
| Patient 13 | 663.21 | 558.16 | 638.25 | 471.4 | 331.35 | 510.58 | 418.1 | 532.02 |
| Patient 14 | 118.24 | 114.37 | 146.33 | 84.53 | 16 | 86.05 | 99.76 | 86.05 |
| Patient 15 | 16 | 2.77 | 0.68 | 0.24 | 417.15 | 24.55 | 80 | 49.93 |
| Patient 16 | 105.98 | 182.56 | 82.76 | 45.29 | 81.84 | 121.04 | 83.68 | 49.93 |
| Patient 17 | 1143 | 847.94 | 92.92 | 478.94 | 178.67 | 1367 | 1115 | 546.47 |
| Patient 18 | 3637 | 2585 | 1851 | 2048 | 1744 | 1882 | 2581 | 2687 |
| Patient 19 | 1377 | 1368 | 433.52 | 235.72 | 309.98 | 199.19 | 388.66 | 743.34 |
| Patient 20 | 151.3 | 254.91 | 166.22 | 173.79 | 228.89 | 175.06 | 184.01 | 82.13 |
| Patient 21 | 459.22 | 516.81 | 141.54 | 123.66 | 301.59 | 442.43 | 559.33 | 606.72 |
| Patient 22 | 711.7 | 483.12 | 682.21 | 931.37 | 735.45 | 520.54 | 537.72 | 562.88 |
| Patient 23 | 264.66 | 148.85 | 123.66 | 348.5 | 110.91 | 90.77 | 7.4 | 1.01 |
| Patient 25 | 107.49 | 4.1 | 91.86 | 121.32 | 259.08 | 415.87 | 116.67 | 13.26 |
| Patient 26 | 106.96 | 99.94 | 129.09 | 90.87 | 90.87 | 88.65 | 90.87 | 99.94 |
| Patient 27 | 2.05 | 16 | 150.07 | 104.6 | 90.87 | 119.06 | 7.39 | 2.57 |
| Patient 29 | 0.47 | 1.28 | 4.91 | 0.79 | 3.2 | 2.05 | 3.2 | 1.28 |
| Patient 30 | 64.31 | 51.51 | 97.65 | 16 | 41.11 | 25.86 | 80 | 41.11 |
| Patient 31 | 228.95 | 166.91 | 230.84 | 186.52 | 146.08 | 134.22 | 154.69 | 158.16 |
| Patient 34 | 677.97 | 440.76 | 223.31 | 479.05 | 567.73 | 456.17 | 610.42 | 641.29 |
| Patient 30 | 712 | 533.98 | 696.14 | 512.83 | 286.95 | 563.88 | 677.29 | 675.25 |
| Average | 967.3673 | 784.8 | 567.3766 | 705.2203 | 734.1513 | 784.4797 | 881.2959 | 719.4634 |

P values VEGF, n = 30 (p < 0.05 = statistically significant (*)):
after surgery vs. before surgery = 0.07035* (down);
Day 1 vs. before surgery = 0.0816* (down);
Day 2 vs. before surgery = 0.0288* (down);
Day 3 vs. before surgery = 0.05952* (down);
Day 7 vs. before surgery = 0.06271* (down);
Day 14 vs. before surgery = 0.08025* (down);
Day 28 vs. before surgery = 0.1007

Table 16 shows that EGF levels are down-regulated at Day 1 and Day 2 after surgery.

TABLE 16
Significant down-regulation of EGF levels at Day 1 and Day 2 after surgery.

| EGF | Before | After | D1 | D2 | D3 | D7 | D14 | D28 |
|---|---|---|---|---|---|---|---|---|
| Patient 1 | 0.91 | 1.11 | 0.31 | 0.74 | 0.39 | 0.74 | 0.66 | 0.62 |
| Patient 2 | 3.29 | 7.53 | 1.89 | 5.24 | 5.49 | 3.2 | 3.49 | 2.04 |
| Patient 3 | 11.79 | 32.02 | 22.74 | 23.84 | 15.49 | 34.47 | 22.53 | 21.62 |
| Patient 4 | 14. | 11.37 | 14.27 | 17.23 | 11.16 | 6.1 | 7 | 8.49 |
| Patient 5 | 642.41 | 351.6 | 230.31 | 367.6 | 388.27 | 514.1 | 393.98 | 424.25 |
| Patient 6 | 1.2 | 3.49 | | 0.85 | 0.99 | 1.15 | 2.04 | 1.75 |
| Patient 7 | 2.5 | 2.42 | 1.75 | 1.89 | 2.34 | 2.84 | 42.22 | 3.58 |
| Patient 8 | 43.28 | 29.13 | 5.36 | 2.84 | 2.75 | 12.86 | 16 | 4.1 |
| Patient 9 | 40.43 | 25.39 | 10.8 | 28.66 | 76.22 | 36.63 | 31.58 | 41.4 |
| Patient 10 | 25.8 | 20.91 | 22.95 | 22.04 | 26.13 | 27.45 | 31.61 | 28.48 |
| Patient 11 | 35.17 | 0.9 | 0.63 | 0.49 | 0.42 | 0.22 | 0.33 | 0.57 |
| Patient 12 | 8.59 | 7.84 | 1.23 | 0.63 | 1.27 | | | |
| Patient 13 | 47.17 | 35.85 | 44.93 | 33.52 | 25.01 | 35.06 | 26.56 | 39.73 |
| Patient 14 | 9.55 | 7 | 7.55 | 21.92 | 29.1 | 18.08 | 18.71 | 22.05 |
| Patient 15 | 3.04 | 1.57 | 0.82 | 0.69 | 0.56 | 1.13 | 1.56 | 2.02 |
| Patient 16 | 10.65 | 21.34 | 5.12 | 6.34 | 5.45 | 16.34 | 5.45 | 5.28 |
| Patient 17 | 260.5 | 230.85 | 69.54 | 152.28 | 354.99 | 255.33 | 223.53 | 120.95 |
| Patient 18 | 34.94 | 20.93 | 4.22 | 16.6 | 16.86 | 30.06 | 17.79 | 23.42 |
| Patient 19 | 85.72 | 92.11 | 71.11 | 66 | 69.92 | 52.79 | 145.14 | 136.11 |
| Patient 20 | 11.36 | 24.3 | 15.36 | 17.72 | 21.39 | 19.99 | 18.14 | |
| Patient 21 | 34.58 | 28.08 | 14.43 | 8.76 | 23.18 | 36.39 | 40.34 | 37.28 |
| Patient 22 | 60.42 | 33.27 | 59.22 | 78.23 | 63.01 | 48.36 | 46.89 | 43.64 |
| Patient 23 | 26.6 | 19.9 | 17.46 | 30.54 | 17.8 | 12.42 | 7.15 | |
| Patient 25 | 11.62 | 4.28 | 6.6 | 21.8 | 29.16 | 37.2 | 18.4 | |
| Average | 59.41792 | 42.21625 | 27.33043 | 38.60208 | 49.47292 | 52.30043 | 48.74348 | 48.369 |

P values EGF, n = 24 (p < 0.05 = statistically significant (*)):
after surgery vs. before surgery = 0.0857;
Day 1 vs. before surgery = 0.04162* (down);
Day 2 vs. before surgery = 0.05083* (down);
Day 3 vs. before surgery = 0.20491;
Day 7 vs. before surgery = 0.07448;
Day 14 vs. before surgery = 0.13942;
Day 28 vs. before surgery = 0.07131

Table 17 shows that MCP-2 levels are down-regulated at Day 1 and Day 2 after surgery.

TABLE 17
Significant down-regulation of MCP levels at Day 1 and Day 2 after surgery.

| MCP-2 | Before | After | D1 | D2 | D3 | D7 | D14 | D28 |
|---|---|---|---|---|---|---|---|---|
| Patient 1 | 22.7 | 22.52 | 17.97 | 24.08 | 20.66 | 22.64 | 19.96 | 21.7 |
| Patient 2 | 19.15 | 20.89 | 16.88 | 21.22 | 20.72 | 17.08 | 19.15 | 13.43 |
| Patient 3 | 18.54 | 31.36 | 25.23 | 21.34 | 17.76 | 27.48 | 26.38 | 22.81 |
| Patient 4 | 22.47 | 20.47 | 16.78 | 18.4 | 20.85 | 21.1 | 16.7 | 15.41 |
| Patient 5 | 20.09 | 18.54 | 12.98 | 17.83 | 23.75 | 27.48 | 24.29 | 25.43 |
| Patient 6 | 22.03 | 35.13 | | 17.47 | 23.43 | 22.35 | 28.12 | 20.72 |
| Patient 7 | 13.43 | 15.44 | 6.37 | 7.05 | 7.05 | 11.19 | 35.33 | 18.24 |
| Patient 8 | 17.86 | 13.19 | 15.01 | 10.66 | 9.82 | 20.21 | 26.69 | 24.75 |
| Patient 9 | 28.75 | 26.01 | 23.28 | 30.21 | 24.28 | 18.11 | 20.55 | 25.62 |
| Patient 10 | 22.75 | 18.54 | 16.7 | 20.79 | 21.76 | 16.23 | 26.56 | 21.55 |
| Patient 11 | 20.55 | 15.31 | 11.54 | 12.76 | 10.89 | 11.54 | 16.13 | 15.81 |
| Patient 12 | 14.97 | 18.96 | 9.74 | 7.69 | 15.31 | | | |
| Patient 13 | 29.59 | 44.14 | 33.98 | 18.11 | 20.93 | 23.52 | 17.82 | 23.52 |
| Patient 14 | 17.07 | 13.53 | 8.49 | 19.51 | 17.82 | 16.45 | 17.07 | 15.97 |
| Patient 15 | 15.47 | 17.23 | 12.76 | 6.83 | 5.4 | 8.24 | 6.39 | 8.09 |
| Patient 16 | 6.12 | 7.3 | 5.11 | 5.67 | 6.04 | 7.7 | 6.98 | 6.98 |
| Patient 17 | 5.95 | 6.81 | 5.95 | 6.3 | 6.81 | 5.49 | 6.56 | 6.12 |
| Patient 18 | 12.9 | 12.44 | 4.62 | 5.58 | 8.39 | 12.21 | 11.91 | 12.96 |
| Patient 19 | 6.89 | 7.38 | | 7.93 | 5.67 | 8.16 | 9.67 | 10.01 |
| Patient 20 | 21.18 | 24.8 | 16.08 | 19.28 | 21.27 | 18.68 | 19.77 | |
| Patient 22 | 12.56 | 10.82 | 9.71 | 9.38 | 17.42 | 12.56 | 9.05 | 11.71 |
| Patient 23 | 23.02 | 15.02 | 14.53 | 11.27 | 14.9 | 11.71 | 9.38 | |
| Patient 25 | 12.7 | 10.04 | 7.99 | 19.28 | 24.17 | 19.67 | 12.97 | |
| Average | 17.68435 | 18.51609 | 13.89048 | 14.72348 | 15.87391 | 16.35455 | 17.61045 | 16.88579 |

P values MCP-2, n = 23 (p < 0.05 = statistically significant (*)):
after surgery vs. before surgery = 0.24955;
Day 1 vs. before surgery = 8.59162E−05* (down);
Day 2 vs. before surgery = 0.00421* (down);
Day 3 vs. before surgery = 0.05392;
Day 7 vs. before surgery = 0.11152;
Day 14 vs. before surgery = 0.45277;
Day 28 vs. before surgery = 0.23071

Table 18 shows that SDF1AB levels are down-regulated at Day 1 after surgery.

TABLE 18
Significant down-regulation of SDF1AB levels at Day 1 after surgery.

| SDF1AB | Before | After | D1 | D2 | D3 | D7 | D14 | D28 |
|---|---|---|---|---|---|---|---|---|
| Patient 1 | 3887 | 3891 | 3258 | 3375 | 4393 | 4698 | 4377 | 4433 |
| Patient 2 | 2308 | 2832 | 1278 | 2525 | 2344 | 1794 | 1359 | 1438 |
| Patient 3 | 5068 | 5182 | 5019 | 5725 | 5108 | 7173 | 6972 | 5652 |
| Patient 4 | 3814 | 4341 | 3262 | 5001 | 4756 | 3665 | 2948 | 3652 |
| Patient 5 | 9279 | 8607 | 7981 | 10837 | 9442 | 10108 | 9475 | 10983 |
| Patient 6 | 3971 | 4185 | | 4061 | 4105 | 4602 | 4263 | 3704 |
| Patient 7 | 2776 | 2951 | 2754 | 2393 | 2821 | 2571 | 4535 | 2832 |
| Patient 8 | 2595 | 2641 | 2821 | 2962 | 3551 | 3980 | 5084 | 3434 |
| Patient 9 | 2026 | 2066 | 1261 | 1822 | 1561 | 1815 | 1870 | 1590 |
| Patient 10 | 4317 | 4047 | 4163 | 4417 | 4457 | 4492 | 3244 | 2962 |
| Patient 11 | 3341 | 2819 | 2819 | 3493 | 2736 | 2164 | 2970 | 2889 |
| Patient 12 | 2356 | 2688 | 1829 | 2072 | 1952 | | | |
| Patient 13 | 4477 | 4677 | 4525 | 4429 | 4667 | 5352 | 4213 | 4742 |
| Patient 14 | 3005 | 3118 | 1815 | 4094 | 5405 | 3736 | 4003 | 4322 |
| Patient 15 | 2543 | 2394 | 2293 | 2807 | 920.68 | 1141 | 933.83 | 1203 |
| Patient 16 | 1669 | 1973 | 1628 | 2263 | 1985 | 1735 | 1494 | 1833 |
| Patient 17 | 1589 | 1510 | 1432 | 1669 | 1465 | 1682 | 1639 | 1641 |
| Patient 18 | 1156 | 1176 | 727.28 | 1027 | 950.15 | 1287 | 1110 | 1427 |
| Patient 19 | 2605 | 2480 | 2476 | 2416 | 1715 | 1602 | 2693 | 2371 |
| Patient 20 | 2104 | 2545 | 2286 | 2710 | 2384 | 2166 | 2335 | |
| Patient 21 | 4415 | 2764 | 2912 | 3222 | 2755 | 4027 | 3523 | 2834 |
| Patient 22 | 3040 | 2479 | 2498 | 2145 | 2216 | 3222 | 2554 | 2825 |
| Patient 23 | 4005 | 3359 | 2441 | 3181 | 2904 | 2246 | 2470 | |
| Patient 25 | 1299 | 1153 | 822.88 | 807.63 | 2063 | 2508 | 1792 | |
| Average | 3235.208 | 3161.583 | 2708.746 | 3310.368 | 3193.993 | 3381.13 | 3298.123 | 3338.35 |

P values SDF1AB, n = 24 (p < 0.05 = statistically significant (*));
after surgery vs. before surgery = 0.22973;
Day 1 vs. before surgery = 8.0633E−05* (down);
Day 2 vs. before surgery = 0.29111;
Day 3 vs. before surgery = 0.40954;
Day 7 vs. before surgery = 0.29029;
Day 14 vs. before surgery = 0.45502;
Day 28 vs. before surgery = 0.38572

Table 19 shows that PDGFAA levels are down-regulated at Day 1 and Day 3 after surgery.

TABLE 19
Significant down-regulation of PDGFAA levels at Day 1 and Day 3 after surgery.

| PDGFAA | Before | After | D1 | D2 | D3 | D7 | D14 | D28 |
|---|---|---|---|---|---|---|---|---|
| Patient 1 | 229.43 | 476.43 | 241.85 | 363.24 | 330.51 | 309.88 | 372.21 | 276.43 |
| Patient 2 | 217.79 | 525.7 | 107.2 | 349.6 | 244.96 | 198.02 | 154.28 | 154.28 |
| Patient 3 | 381.95 | 2058 | 1272 | 1160 | 500.67 | 2816 | 1167 | 1012 |
| Patient 4 | 413.16 | 547.21 | 458.07 | 549.95 | 544.87 | 412.77 | 271.76 | 279.54 |
| Patient 5 | 551.17 | 499.84 | 196.12 | 532.87 | | 1201 | 1000 | 697.24 |
| Patient 6 | 1193 | 2347 | | 839.09 | 1641 | 1225 | 1947 | 1458 |
| Patient 7 | 379.73 | 307.55 | 54.35 | 90.3 | 118.99 | 132.59 | 1201 | 656.66 |
| Patient 8 | 120.8 | 195.19 | 50.91 | 42.36 | 46.62 | 299.91 | 727.4 | 274.2 |
| Patient 9 | 1061 | 867.54 | 180.34 | 1367 | 211.98 | 569.5 | 422.47 | 177.56 |
| Patient 10 | 253.89 | 369.48 | 378.44 | 496.75 | 713.36 | 117.99 | 436.33 | 698.68 |
| Patient 11 | 321.8 | 262.87 | 455.05 | 222.62 | 132.02 | 109.34 | 108.81 | 141.88 |
| Patient 12 | 365.44 | 705.9 | 211.1 | 90.3 | 144.08 | | | |
| Patient 13 | 96.6 | 195.08 | 57.09 | 41.92 | 241.78 | 658.83 | 145.18 | 554.95 |
| Patient 14 | 309.19 | 442.56 | 40.96 | 1420 | 320.6 | 505.36 | 1262 | 754.64 |
| Patient 15 | 914.37 | 440.69 | 336.89 | 107.2 | 183.17 | 667.3 | 718.96 | 627.25 |
| Patient 16 | 404.28 | 702.02 | 217.99 | 225.8 | 297.26 | 602.37 | 292.89 | 236.23 |
| Patient 17 | 141.1 | 209.31 | 162.62 | 176.43 | 246.67 | 117.94 | 115.38 | 183.34 |
| Patient 18 | 831.5 | 1368 | 119.65 | 248.41 | 834.18 | 3912 | 1688 | 1590 |
| Patient 19 | 1058 | 1239 | 303.38 | 1185 | 676.19 | 1255 | 1307 | 1524 |
| Patient 20 | 416.5 | 1003 | 701.93 | 670.83 | 972.98 | 746.38 | 678 | 519.96 |
| Patient 21 | 1298 | 385.05 | 150.77 | 131.89 | 418.79 | 2157 | 822.56 | 694.74 |
| Patient 22 | 781.37 | 609 | 397.61 | 89.79 | 285.37 | 297.62 | 247.24 | 717.52 |
| Patient 23 | 1796 | 1182 | 972.98 | 2035 | 852.36 | 479.98 | 394.75 | 473 |
| Patient 25 | 283.14 | 139.73 | 76.74 | 240.65 | 700.13 | 1397 | 338.56 | 289.86 |
| Patient 26 | 421.61 | 581.44 | 299.9 | 486.77 | 430.25 | 441.43 | 490.33 | 523.03 |
| Patient 27 | 303.92 | 297.89 | 129.28 | 291.37 | 267.83 | 828.3 | 197.65 | 428.22 |
| Patient 29 | 165.07 | 343.17 | 269.33 | 60.73 | 67.41 | 444.48 | 220.47 | 367.4 |
| Patient 30 | 1076 | 706.05 | 457.71 | 637.47 | 654.98 | 537.87 | 251.84 | 518.94 |
| Patient 31 | 779.81 | 1707 | 854.63 | 1100 | 871.93 | 929.66 | 1174 | 1432 |
| Patient 33 | 1728 | 1120 | 172.22 | 1218 | 1474 | 1350 | 1298 | 757.41 |
| Patient 34 | 523.33 | 309.95 | 411.98 | 254.33 | 369.82 | 1253 | 1155 | 401.85 |
| Patient 35 | 324.47 | 245.47 | 82.82 | 787.54 | 304.38 | 71.08 | 66.29 | 57.29 |
| Average | 598.1694 | 699.66 | 316.8358 | 547.2878 | 487.069 | 840.1484 | 666.8503 | 596.0677 |

P values PDGFAA, n = 32 (p < 0.05 = statistically significant (*)):
after surgery vs. before surgery = 0.13228;
Day 1 vs. before surgery = 0.00187* (down);
Day 2 vs. before surgery = 0.26105;
Day 3 vs. before surgery = 0.54894* (down);
Day 7 vs. before surgery = 0.06234;
Day 14 vs. before surgery = 0.26536;
Day 28 vs. before surgery = 0.45594

Table 20 shows that PDGFBB levels are down-regulated at Day 1 after surgery.

TABLE 20
Significant down-regulation of PDGFBB levels at Day 1 after surgery.

| PDGFBB | Before | After | D1 | D2 | D3 | D7 | D14 | D28 |
|---|---|---|---|---|---|---|---|---|
| Patient 1 | 990.23 | 2865 | 1125 | 3465 | 1388 | 1160 | 1639 | 1023 |
| Patient 2 | 1125 | 3047 | 237.65 | 1309 | 837.88 | 456.84 | 320 | 516.83 |
| Patient 3 | 2424 | 16265 | 10458 | 9518 | 3139 | 25550 | 9585 | 7567 |
| Patient 4 | 1778 | 2620 | 2252 | 2753 | 2664 | 1798 | 958.37 | 958.37 |
| Patient 5 | 5115 | 3251 | 1685 | 3936 | | 14389 | 11141 | 6959 |
| Patient 6 | 7163 | 15197 | | 3304 | 12581 | 5933 | 13607 | 9532 |
| Patient 7 | 2367 | 1994 | 186.04 | 229.34 | 320 | 449.01 | 15393 | 3547 |
| Patient 8 | 365.06 | 723.74 | 139.91 | 123.79 | 176.12 | 826.07 | 5773 | 1155 |
| Patient 9 | 7197 | 5177 | 380.95 | 9167 | 652.67 | 3251 | 1600 | 586.33 |
| Patient 10 | 2402 | 4266 | 3632 | 5791 | 9205 | 867 | 4379 | 7154 |
| Patient 11 | 2452 | 2208 | 3033 | 1714 | 549.72 | 341.17 | 436.31 | 549.72 |
| Patient 12 | 1783 | 3882 | 490.61 | 170.29 | 341.17 | | | |
| Patient 13 | 299.82 | 839.96 | 197.74 | 87.62 | 1668 | 2280 | 262.28 | 2828 |
| Patient 15 | 6449 | 2256 | 1875 | 386.59 | 648.06 | 3426 | 4317 | 3137 |
| Patient 16 | 2787 | 7799 | 1088 | 1138 | 1919 | 4357 | 2132 | 1243 |
| Patient 17 | 340.67 | 1536 | 458.94 | 706.52 | 903.52 | 281.4 | 340.67 | 903.52 |
| Patient 18 | 2617 | 5585 | 263.43 | 781.12 | 2925 | 21090 | 7208 | 7015 |
| Patient 19 | 9563 | 11867 | 2009 | 10745 | 2617 | 7899 | 13652 | 18985 |
| Patient 20 | 2105 | 5366 | 3483 | 2661 | 4585 | 3592 | 2888 | 2228 |
| Patient 21 | 10485 | 2339 | 646.68 | 456.88 | 1654 | 13233 | 4725 | 3702 |
| Patient 22 | 7763 | 5681 | 3924 | 333.71 | 1130 | 1990 | 1728 | 5294 |
| Patient 25 | 986.02 | 440.13 | 268.72 | 1526 | 3924 | 5318 | 1014 | 1092 |
| Patient 26 | 2209 | 3114 | 1366 | 2621 | 2325 | 2423 | 2462 | 2742 |
| Patient 27 | 1635 | 1635 | 348.28 | 2344 | 1834 | 5231 | 1330 | 2762 |
| Patient 29 | 857.99 | 2581 | 857.99 | 211.87 | 155.63 | 1689 | 831.39 | 1671 |
| Patient 30 | 11639 | 6949 | 3475 | 6212 | 5667 | 3891 | 1559 | 3605 |
| Patient 31 | 3128 | 10285 | 2500 | 3831 | 4183 | 3831 | 4678 | 6528 |
| Patient 33 | 9895 | 5331 | 561.17 | 5226 | 6048 | 5331 | 6268 | 2583 |
| Patient 34 | 2583 | 1545 | 1894 | 644.53 | 1545 | 7841 | 7960 | 1672 |
| Patient 35 | 1624 | 1153 | 254.42 | 142.46 | 2527 | 142.46 | 198.98 | 152.71 |
| Average | 3737.593 | 4593.261 | 1692.811 | 2717.857 | 2693.544 | 5133.343 | 4427.103 | 3713.499 |

P values PDGFBB, n = 30 (p < 0.05 = statistically significant (*)):
after surgery vs. before surgery = 0.13346;
Day 1 vs. before surgery = 0.00455* (down);
Day 2 vs. before surgery = 0.05306;
Day 3 vs. before surgery = 0.07732;
Day 7 vs. before surgery = 0.13632;
Day 14 vs. before surgery = 0.24104;
Day 28 vs. before surgery = 0.45003

In a limited number of patients, although not followed longitudinally as with the more exhaustive data provided in Examples 3-5, preliminary data exists to suggest that PGE-2 is upregulated over a similar time period to IL-6, HGF, PDGF, MCP-1 and MMP-9.

An additional 25 patients undergoing surgery for breast, colorectal, and prostate cancer were enrolled. Research efforts for these patients was focussed on the expression patterns of IL-6, HGF, and TGF-beta and a similar statistically significant change from baseline was observed as in the above data set.

### Example 3

### Inflammatory Cytokines Facilitate Cell Proliferation and EMT in Cancer Stem Cell Populations

Specific cytokines or cytokine cocktails capable of inducing cell proliferation of cancer stem cell populations and/or triggering stem cell enrichment via Epithelial Mesenchymal Transition (EMT) and/or stem cell self renewal were identified.

Cell proliferation of cancer and/or cancer stem cell populations with selected cytokine and cytokine cocktails on serum starved colorectal carcinoma cell lines. Prior to incubation cells were stained with a cell proliferation dye, which binds stoichiometrically to DNA. Intensity of the dye decreased upon cell division thereby signifying cell proliferation.

For specific cytokine combinations, there was an enrichment of cells with cancer stem cell (CSC) phenotype. Imaging flow cytometry data show that cells incubated with cytokines were able to resume cell division in the absence of serum. Moreover, specific cytokine combinations achieved a high percentage of stem cell population of the total events collected. Data from enriched Circulating Tumour Cells (CTCs) from a patient and from cell lines support this observation.

Cell Proliferation Assays with Cytokine Cocktails

Two authenticated colorectal carcinoma cell lines, HCT-15 and SW-620, were used to test the effect of different cytokine and cytokine cocktails, including IL-6, HGF, PGE-2, TGF-beta and MMP-9 alone or in a cocktail, on cell proliferation, EMT and cancer stem cell populations.

Table 21 shows the cytokine and cytokine cocktail treatments used in cell proliferation assays.

TABLE 21
Cytokine and Cytokine Cocktail Treatments
for Cell Proliferation Assay

| Source | 2 Component | 3 Component | 5 Component |
|---|---|---|---|
| of Cells | Cocktail | Cocktail | Cocktail |
| SW620 | IL6 + HGF | IL6 + HGF + PGE2 | IL6 + HGF + |
| | | | PGE2 + TGF + |
| | | | MMP9 |
| | IL6 + PGE2 | IL6 + HGF + TGF | |
| | IL6 = MMP9 | IL6 + PGE2 + TGF | |
| | | IL6 + PGE2 + | |
| | | MMP9 | |
| | | MMP9 + HGF + TGF | |

In order to evaluate stem cell populations, cells were synchronized by serum starvation for 72 hours before seeding in 24-well ultra low attachment plates at a density of 200,000 cells/well. Cells were then detached using trypsin EDTA and washed twice with PBS (without Calcium or Magnesium). A cell proliferation dye, eFluor 450 was used to label cells. Cells were grown with DMEM with high glucose and 2 mM Gluatmine without FBS in the presence of selected cytokines and or cytokine cocktails over a period of 72 hrs.

Cells that were grown in 5% serum were stained with the following antibody cocktails: CD24FITC, CD44PE and CD24FITC, CD133PE. Cells were also stained with a fixable live/dead efluor dye 506 to exclude dead cells. In parallel, cells were grown in the presence of 5% serum to evaluate CSC markers.

Cells were fixed in 1% Paraformaldehyde for 15 minutes at room temperature and run on Flowsight using 2 lasers (488 nm at 60 mW and 405 nm at 30 mW). For cell lines, 100,000 events were collected whilst for enriched CTCs all events were collected until the sample finished.

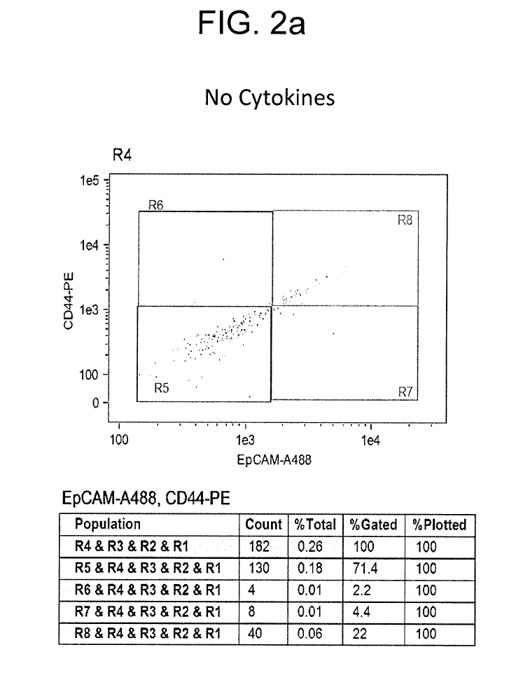

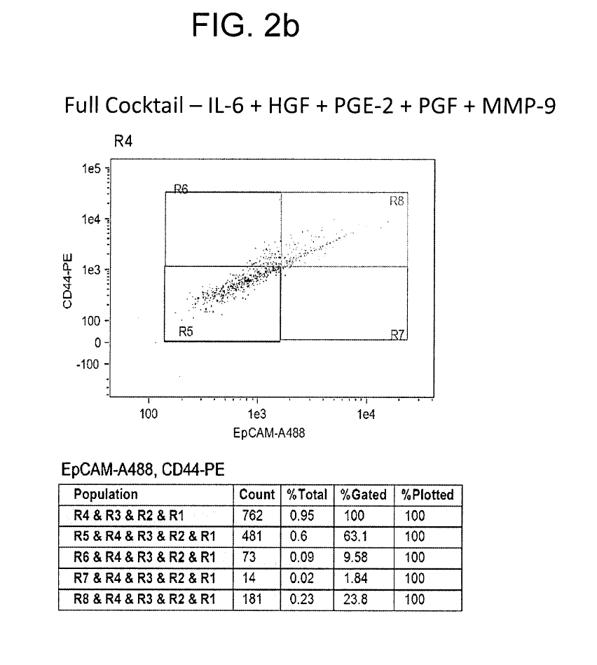

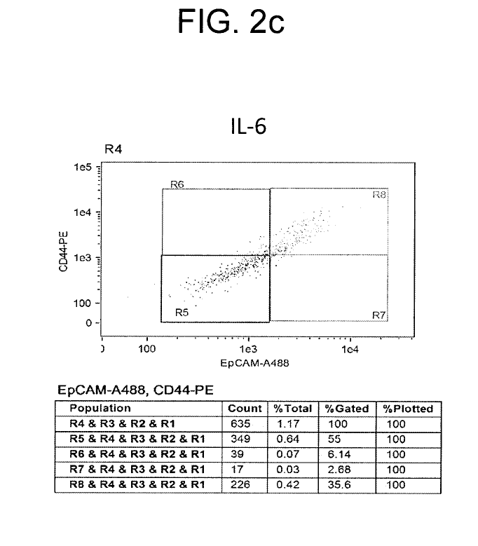

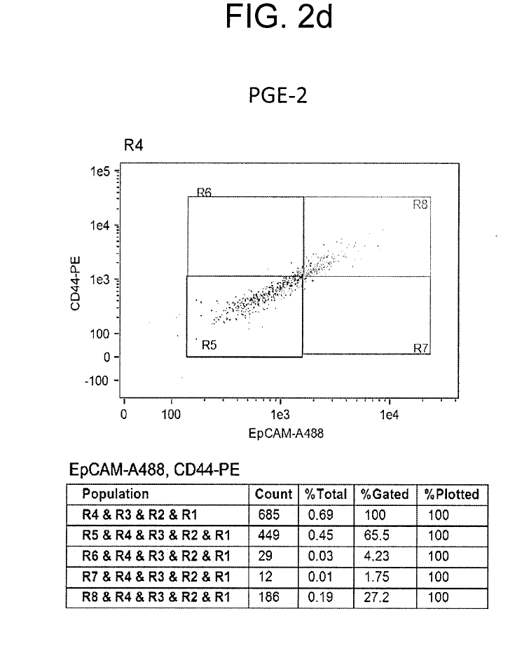

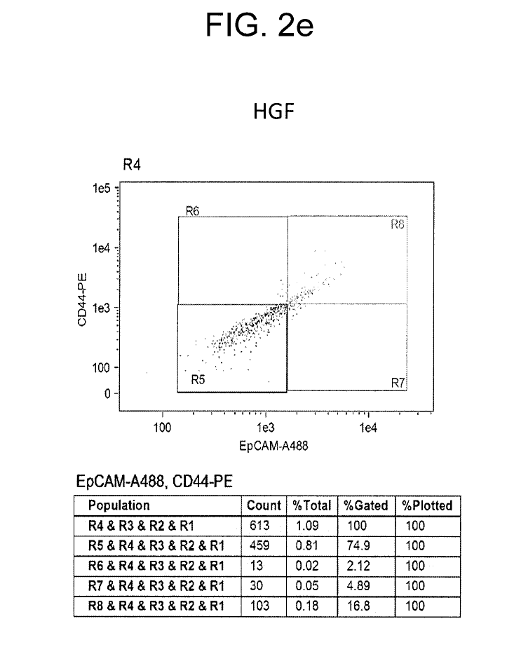

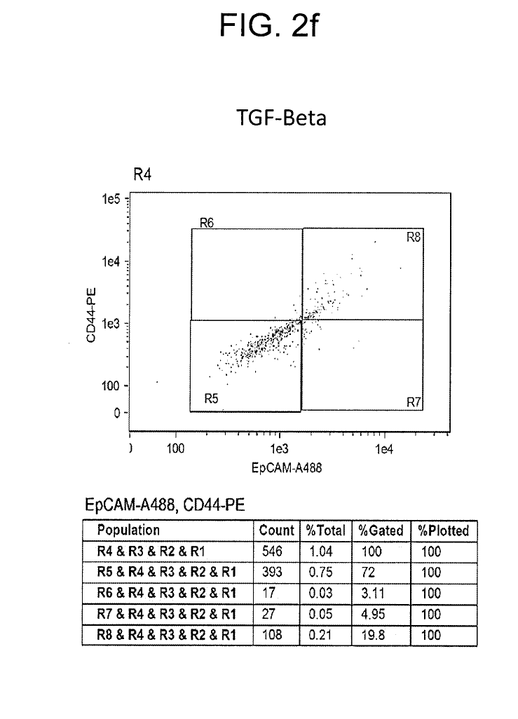

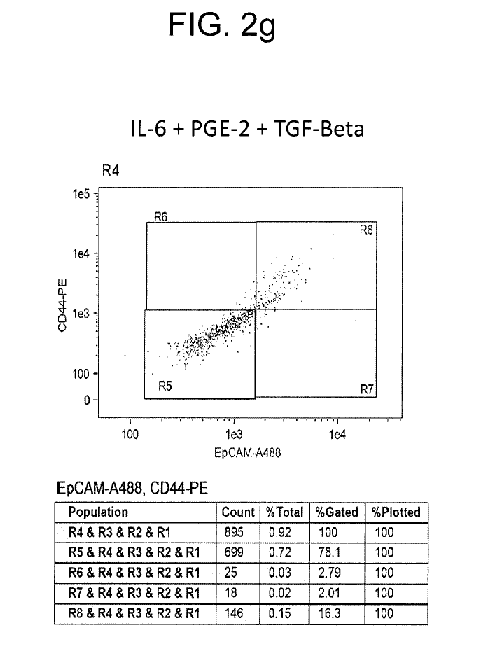

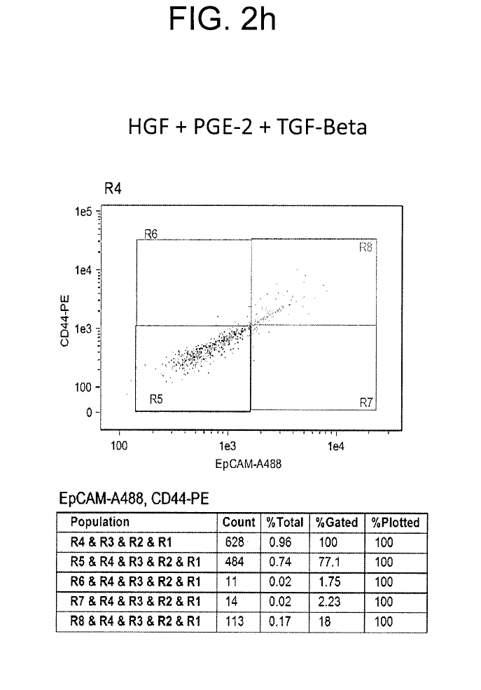

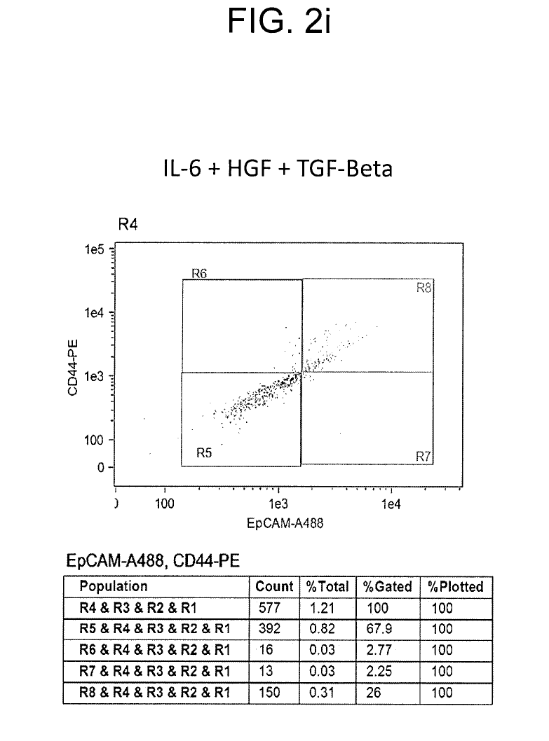

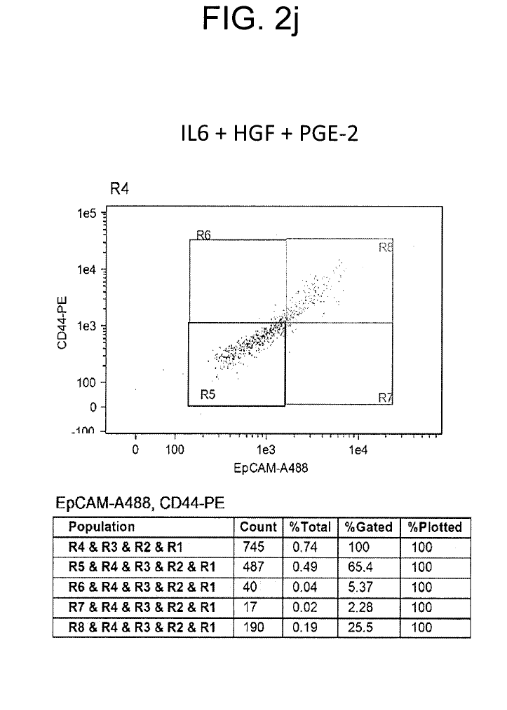

Flow cytometry data was analysed using IDEAS software. Double events, out of focus events, CD45PerePCy5.5 positive cells (in case of enriched CTCs), dead cells and non-nucleated events were eliminated from the fluorescence analysis of the cell population. FIG. 2 depicts scatterplots of flow cytometry experiments showing the enriched CTCs stained with an antibody containing CD44 after treatment with various cytokines and cytokine cocktails. Hoescht 33258 dye was used to stain nuclei in the case of the HCT-15 cell line and enriched CTCs. All experiments were stained with a fixable live/dead dye eFluor 506 to exclude dead cells.

CTC Enrichment and Cell Culture with Cytokine Cocktails

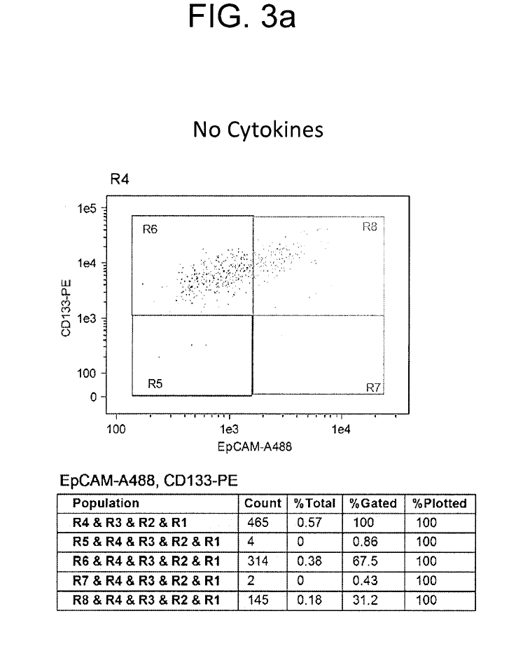

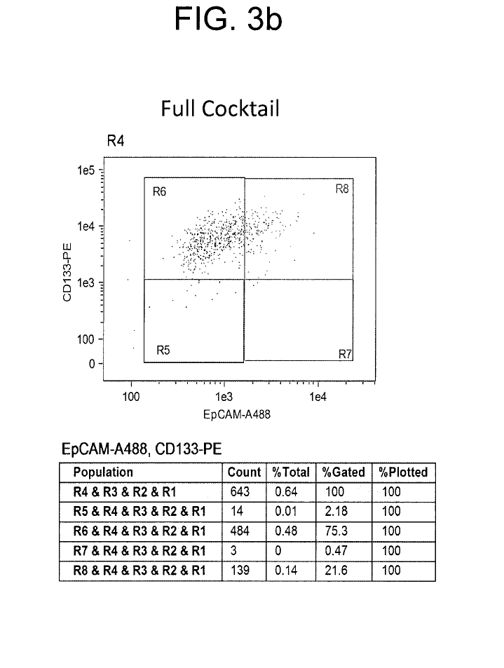

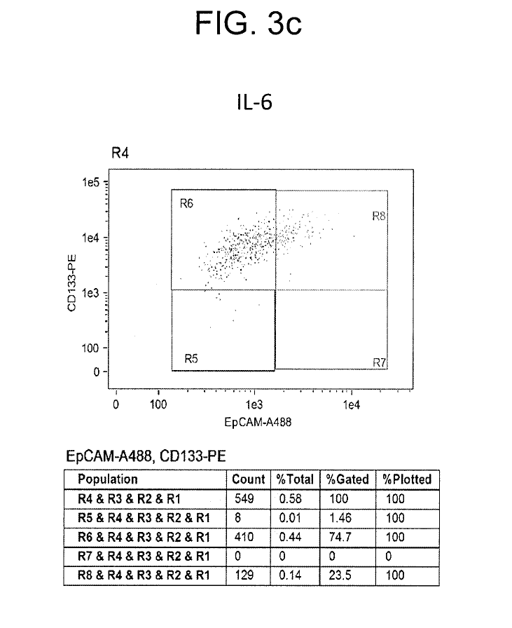

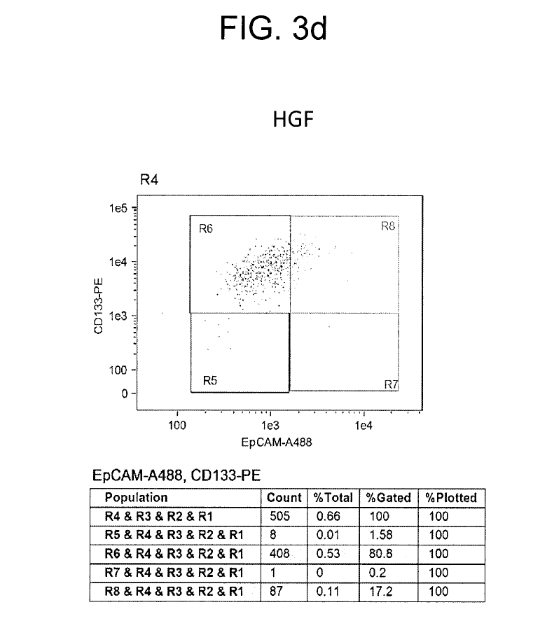

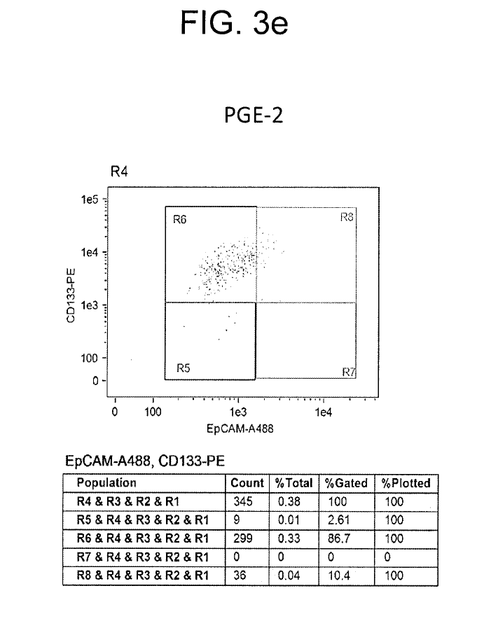

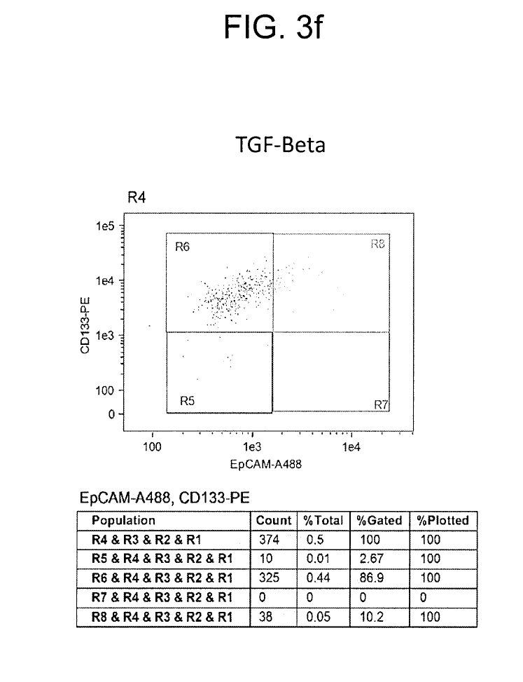

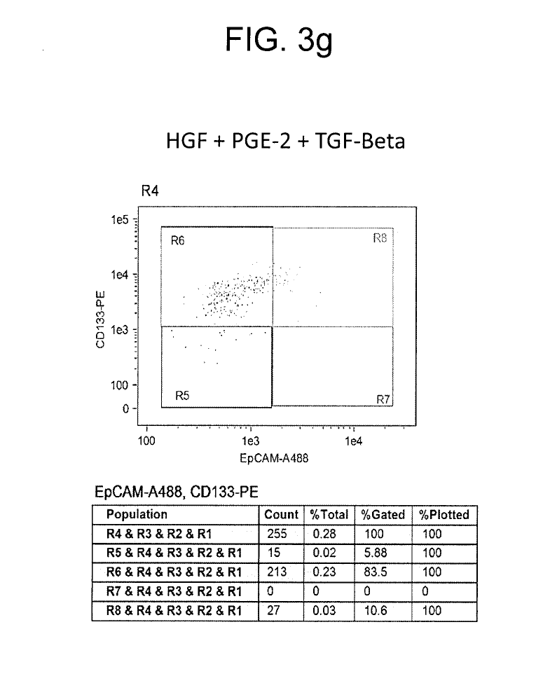

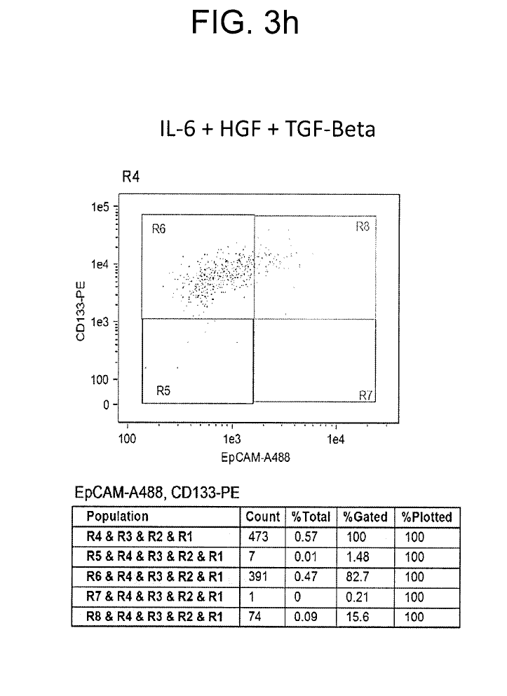

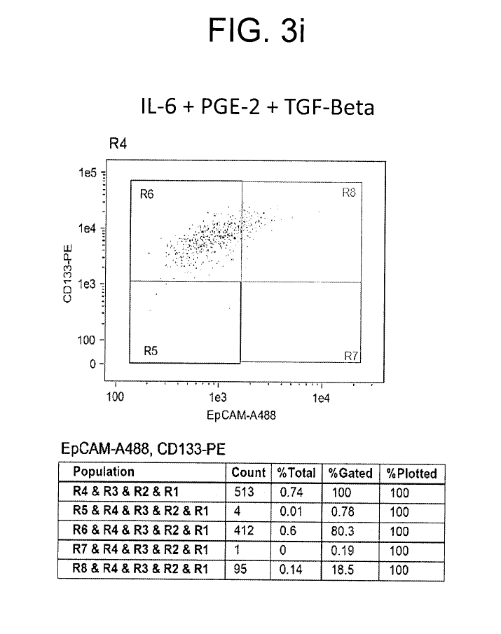

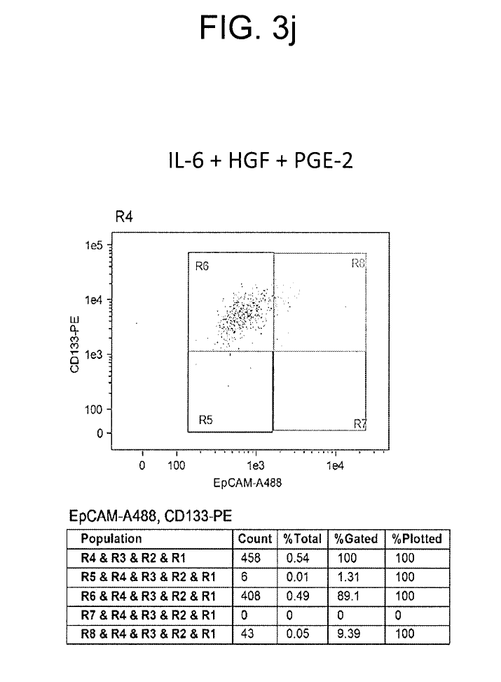

In order to determine if selected cytokines influence cell proliferation of circulating tumour cells (CTCs), whole blood was collected from a patient with metastatic prostate cancer. Circulating tumour cells were enriched from whole blood using RosetteSep CTC Enrichment cocktail. Briefly, samples of whole blood were incubated with RosetteSep (anti CD 56 for 20 minutes at room temperature). Phosphate buffered saline containing 2% Fetal Bovine Serum was added to the samples and layered on a Ficoll Paque gradient. After centrifugation, the supernatant which contained CTCs was washed twice with PBS containing 2% FBS. Equal volumes of the cell suspension were seeded into a 24-well ultra-low attachment polystyrene plate. Enriched CTCs were cultures in RPMI 1640 medium with 2 mM glutamine and no serum over a period of 8 days. Different cytokines and/or cytokine cocktails were added to the medium as shown in Table 22. FIG. 3 depicts scatterplots of flow cytometry experiments enriched CTCs stained with an antibody containing CD133 after treatment with various cytokines and cytokine cocktails.

TABLE 22
Cytokine and Cytokine Cocktail Treatments for Enriched CTCs

| Source | 1 Component | 3 Component | 4 Component |
|---|---|---|---|
| of Cells | Cocktail | Cocktail | Cocktail |
| Peripheral | IL6 | IL6 + HGF + PGE2 | IL6 + HGF + |
| Blood | | | PGE2 + TGF |
| | HGF | IL6 + HGF + TGF | |
| | PGE2 | IL6 + PGE2 + TGF | |
| | TGF | HGF + PGE2 + TGF | |

Single component, 2-, 3-, 4- and 5-component cytokine cocktails were evaluated with respect to cell proliferation and expression of cancer stem cell-like markers. These investigations were performed in both cell lines and in circulating tumour cells enriched from whole blood of a patient with metastatic prostate cancer.

Results show that some cytokine cocktails encourage cell proliferation. Results on cell proliferation in cell lines and enriched CTCs are presented. In both cases, cells were grown in serum-free media. Results are also presented showing the effect the cytokine cocktails on cell lines and cultured CTCs with respect to cancer stem cell-like phenotypes. For these experiments, cells were grown in media supplemented with 5% FBS.

Cell Proliferation in the HCT-15 Cell Line

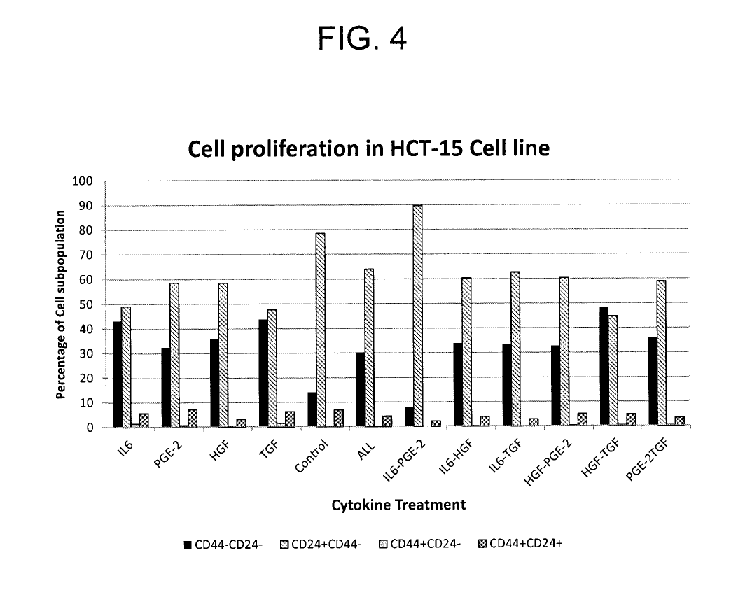

To evaluate which cocktail combinations were most likely involved in cell proliferation of presumed quiescent stem cells or de-differentiated stein cell-like cells, four cytokines, IL-6, HGF, PGB-2 and TO were added singly or in pairs to HCT-15 cells. Results of combinations that influenced cell proliferation of HCT-15 cells in culture are presented. Table 23 shows cell subpopulations after exposure to various cytokines and cytokine combinations. FIG. 4 depicts cell proliferation of subpopulations in the HCT-15 cell line after exposure to various cytokines and cytokine cocktails.

TABLE 23
Cell Subpopulations after exposure to cytokine combinations

| | CD44−CD24− | CD24+CD44− | CD44+CD24− | CD44+CD24+ |
|---|---|---|---|---|
| IL6 | 43.2 | 49.1 | 1.35 | 5.63 |
| PGE-2 | 32.4 | 58.7 | 0.61 | 7.3 |
| HGF | 35.9 | 58.6 | 0.31 | 3.29 |
| TGF | 43.8 | 47.7 | 1.51 | 6.26 |
| Control | 14.1 | 78.5 | 0.14 | 6.9 |
| ALL | 30.3 | 64 | 0.09 | 4.25 |
| IL6-PGE-2 | 7.85 | 89.7 | 0 | 2.17 |
| IL6-HGF | 33.9 | 60.4 | 0.26 | 3.96 |
| IL6-TGF | 33.4 | 62.7 | 0.06 | 2.91 |
| HGF-PGE-2 | 32.8 | 60.4 | 0.37 | 5.09 |
| HGF-TGF | 48.3 | 44.8 | 0.43 | 4.89 |
| PGE-2-TGF | 35.8 | 58.9 | 0.28 | 3.45 |

The CSC markers CD44 and CD24 have been used to characterize cancer stem cells among others as various cancer stem cell markers in various cell lines including HCT-15 and SW-620 (Muraro et al. 2012). In their studies, Muraro and coworkers showed that 94.87% of HCT-15 cells were double negative when stained with similar antibodies (CD44 and CD24). The cytokine combination of IL-6 and PGE-2 appears to influence significantly the expression of CD-24 as shown in FIG. 4. A 2-component cytokine combination of IL-6 and PGE-2 increased significantly the CD24+CD44-population compared with the control where no cytokines were added.

Cell Proliferation in the SW-620 Cell Line

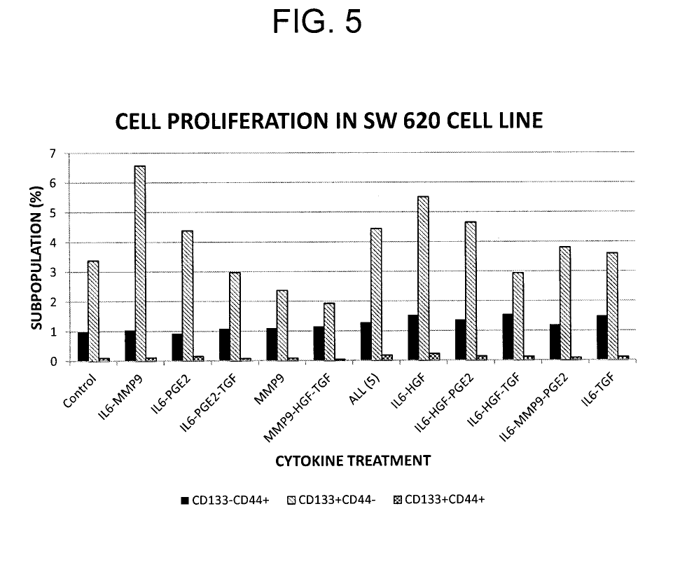

To determine cell proliferation upon the addition of cytokines, SW-620 cells were synchronized by serum starvation for 72 hours. The cells were harvested and stained with a cell proliferation dye before being exposed to selected cytokine combinations. Cells were grown over a period of 72 hours without serum to assess the specific contribution of cytokines in cell proliferation. Results are presented to show which subpopulations of cells show cell proliferation. Table 24 shows cell proliferation of cell subpopulations after exposure to various cytokines and cytokine cocktails. FIG. 5 depicts cell proliferation of subpopulations in the SW 620 cell line after exposure to various cytokines and cytokine cocktails.

TABLE 24
Cell Proliferation in Subpopulations after cytokine treatment

| | CD133−CD44− | CD133−CD44+ | CD133+CD44− | CD133+CD44+ |
|---|---|---|---|---|
| Control | 95.3 | 0.99 | 3.33 | 0.1 |
| IL6-MMP9 | 92 | 1.04 | 6.58 | 0.11 |
| IL6-PGE2 | 94.3 | 0.92 | 4.39 | 0.15 |
| IL6-PGE2-TGF | 95.7 | 1.08 | 2.97 | 0.08 |
| MMP9 | 96 | 1.1 | 2.37 | 0.09 |
| MMP9-HGF-TGF | 96.7 | 1.15 | 1.93 | 0.04 |
| ALL (5) | 93.9 | 1.29 | 4.45 | 0.18 |
| IL6-HGF | 92.5 | 1.53 | 5.51 | 0.23 |
| IL6-HGF-PGE2 | 93.5 | 1.37 | 4.65 | 0.14 |
| IL6-HGF-TGF | 95.1 | 1.55 | 2.94 | 0.13 |
| IL6-MMP9-PGE2 | 94.5 | 1.2 | 3.81 | 0.09 |
| IL6-TGF | 94.4 | 1.49 | 3.6 | 0.11 |

Subpopulations are expressed in as a percentage of gated cells

Cells that were not exposed to cytokines show the least proliferation in the stem cell sub-population fraction (0.99% gated cells). The highest proliferation with the 72 hour period was in cells treated with the following cytokine cocktail:

1. IL-6, HGF, TGF (1.55% gated cells)

2. IL-6, HGF (1.55% gated cells)

3. IL-6, TGF (1.49% gated cells)

4. IL-6, HGF, PGE2 (1.37% gated cells)

Selected cytokines were applied to enriched CTCs in culture. Results from these experiments showed cell proliferation with some cytokine combinations.

Cell Proliferation in Enriched CTCs

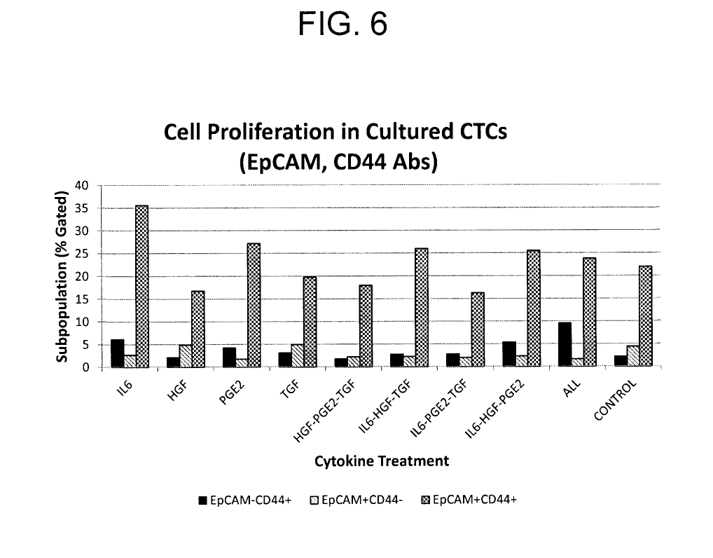

Cells stained with CD44PE antibody cocktail, analysed using flow cytometry had four possible fluorescence staining outcomes: EpCAM positive, CD44 positive, EpCAM and CD44 positive (double positive) and double negative cells (cells negative for both EpCAM and CD44). Table 25 shows cell subpopulations exhibiting cancer stem cell marker after treatment with various cytokines and cytokine cocktails. FIG. 6 depicts cell proliferation of subpopulations in cultured CTCs after exposure to various cytokines and cytokine cocktails. Cells were stained using an antibody containing CD44 PE.

TABLE 25
Cell populations exhibiting cancer stem cell markers after treatment with
different cytokines. Values are expressed as a percentage of gated cells.

| | EpCAM−CD44− | EpCAM−CD44+ | EpCAM+CD44− | EpCAM+CD44+ |
|---|---|---|---|---|
| IL6 | 55 | 6.14 | 2.66 | 35.6 |
| HGF | 74.9 | 2.12 | 4.89 | 16.8 |
| PGE2 | 65.5 | 4.23 | 1.75 | 27.2 |
| TGF | 72 | 3.11 | 4.95 | 19.8 |
| HGF-PGE2-TGF | 77.1 | 1.75 | 2.23 | 18 |
| IL6-HGF-TGF | 67.9 | 2.77 | 2.25 | 26 |
| IL6-PGE2-TGF | 78.1 | 2.79 | 2.01 | 16.3 |
| IL6-HGF-PGE2 | 65.4 | 5.37 | 2.28 | 25.5 |
| ALL | 63.1 | 9.58 | 1.64 | 23.8 |
| CONTROL | 71.4 | 2.2 | 4.4 | 22 |

Numbers represent percentages of gated subpopulations. An antibody cocktail with EpCAM A488, CD133 PE and CD45 PerCPCy5.5 was used. No cell stained positive for CD45 PerCPCy5.5.

CD44 is a marker of stemness in various cancer cells including prostate cancer. Cells treated with all four cytokines had the high cell proliferation of EpCAM-CD44+ cell sub-population during the cell culture period (8 days).

The highest proliferation for this sub-population were in cells which were treated with:

1. all four cytokines; IL-6, HGF, PGE-2 and TGF-beta (9.58% gated)

2. IL-6 (6.14% gated)

3. HGF, PGE2 (5.37% gated)

4. PGE2 (4.23% gated)

Deletion of IL-6 from the cocktail reduced significantly the EpCAM-CD44+ sub-population (1.75% gated compared to 2.2% in cells with no cytokines). This strongly suggests that IL-6 as well as a full cocktail of cytokines is important in cell proliferation of the stem-cell subpopulation (EpCAM-CD44+) of enriched CTCs.

The percentage of cells which stained positive for both EpCAM and CD44 were the highest in cells treated with IL-6. This again strongly suggests that IL-6 is important in cell proliferation of CD44+ cells.

A large number of cells did not stain positive for any antibody (Table 25). These cells were not leucocytes as observed by negative staining of CD45PerCP Cy5.5. To explain this, it is important to look at results of cells from the same samples which were stained with a different antibody cocktail.

It is worth noting that enriched CTCs had a lower counter of EpCAM positive cells as compared to cell lines which had more than 99% EpCAM positive cells.

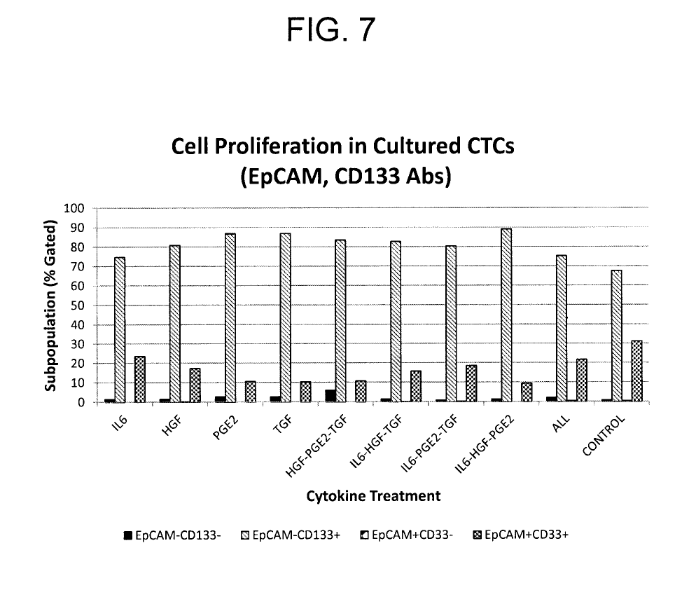

Similarly, cells stained with CD133 PE antibody cocktail, analysed using flow cytometry had four possible fluorescent staining outcomes: EpCAM positive, CD133 positive, EpCAM and CD133 positive (double positive) and double negative (cells negative for both EpCAM and CD133. The different cell populations within a cell population treated by a specific cytokine or cocktail of cytokines. Table 26 shows cell subpopulations exhibiting cancer stem cell marker after treatment with various cytokines and cytokine cocktails. FIG. 7 depicts cell proliferation of subpopulations in cultured CTCs after exposure to various cytokines and cytokine cocktails. Cells were stained using an antibody containing CD133 PE.

TABLE 26
Cell populations exhibiting Cancer Stem Cell Marker
after Treatment with different Cytokines.

| | EpCAM−CD133− | EpCAM−CD133+ | EpCAM+CD133− | EpCAM+CD133+ |
|---|---|---|---|---|
| IL6 | 1.46 | 74.7 | 0 | 23.5 |
| HGF | 1.58 | 80.8 | 0.2 | 17.2 |
| PGE2 | 2.61 | 86.7 | 0 | 10.4 |
| TGF | 2.67 | 86.9 | 0 | 10.2 |
| HGF-PGE2-TFG | 5.88 | 83.5 | 0 | 10.6 |
| IL6-HGF-TGF | 1.48 | 82.7 | 0.21 | 15.6 |
| IL6-PGE2-TGF | 0.78 | 80.3 | 0.19 | 18.5 |
| IL6-HGF-PGE2 | 1.31 | 89.1 | 0 | 9.39 |
| ALL | 2.18 | 75.3 | 0.47 | 21.6 |
| CONTROL | 0.86 | 67.5 | 0.43 | 31.2 |

Numbers represent percentages of gated subpopulations. An antibody cocktail with EpCAM A488, CD133 PE and CD45 PerCPCy5.5 was used. No cell stained positive for CD45 PerCPCy5.5.

CD133 is a marker for stemness in prostate cancer.

The highest proliferation of cells were observed in EpCAM-CD133+ subpopulation. The cytokine cocktail of IL-6, HGF and PGE-2 had the highest EpCAM-CD133+ subpopulation (89.1% gated) and control cells had the lowest gated subpopulation (67.5% gated). The full cocktail surprisingly had a lower subpopulation of EpCAM-CD133+ cell subpopulation. It was expected that the percentages of gated cell subpopulation for EpCAM-CD133+ cell subpopulations would be lower than observed; at least comparable to CD44+ subpopulations. However, CD133 is considered a prominent cancer stem cell marker for prostate cancer whilst CD44 is a cancer stem cell marker for various cancer types.

Moreover in the CD44 cocktail, there was a very high percentage of the double negative subpopulation which could mean that these were CD133+ cells.

Moreover, CTCs appear to respond to cytokine exposure differently from cell lines. One reason could be that CTCs might already be going through the EMT process. The results show that in cell lines more than 99% of cells stained positive for EpCAM whilst in enriched CTCs only about 30% stained positive for EpCAM.

Surprisingly, the highest cell population of double positive was in control cells. Whilst this may be surprising, it may also mean that the absence of cytokines in culture arrested cells in the state of EMT where cells could not progress into cancer stem cells (EpCAM-CD133+).

A very small subpopulation were double negative. This suggests an active EMT process in enriched CTCs.

The cytokine cocktail of IL-6, HGF and PGE-2 appears to enhance cell proliferation both in cell lines and in enriched CTCs. The effect of these cytokines on expression of cancer stem cell markers has varied according to the source of the cells. For cell lines, IL-6-HGF and TGF-beta appeared to be more important whilst in enriched CTCs, IL-6-PGE-2 combinations appeared to be more important.

### Example 4

Evidence of EMT in a Patient with Colorectal Cancer

A blood sample was collected from a patient with colorectal cancer six days after surgery, Blood samples were enriched for CTCs using RosetteSep CD 56 kit according to standard protocol. Prior to cell culture, cells were stained with the following antibody cocktail: EpCAM A488, CD133PE, CD 45 PerCP Cy5.5, and Lgr5-PE-Vio770. Cells were either cultured in low attachment well plates without cytokines (control cells) or with three different cytokines (IL-6, IL-8 or PDGFBB).

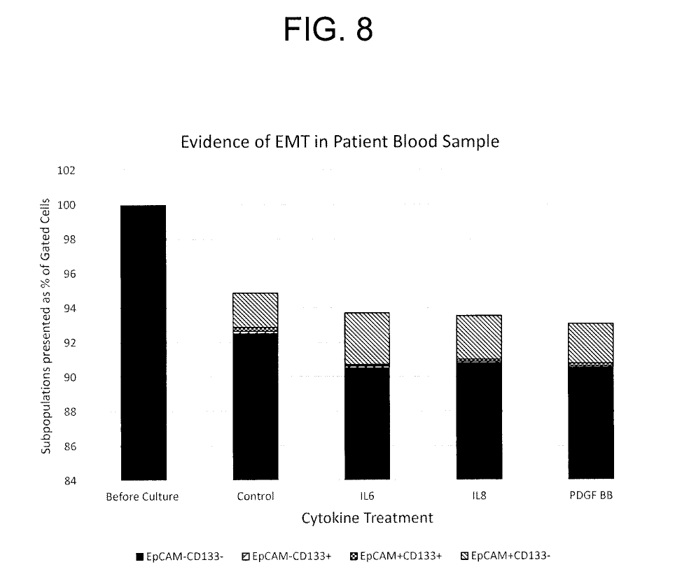

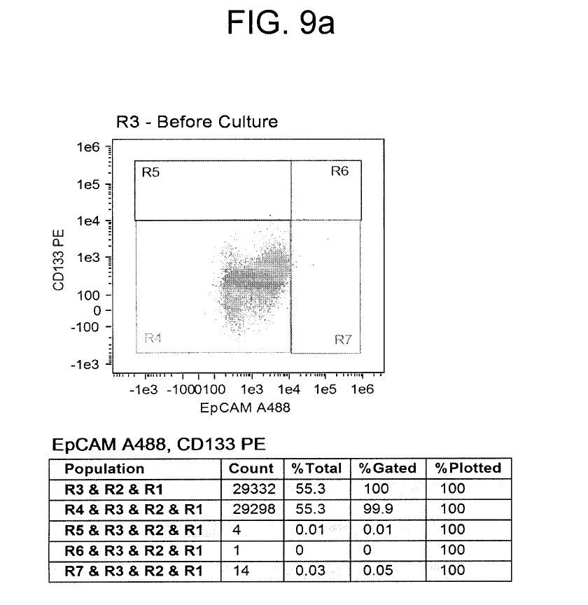

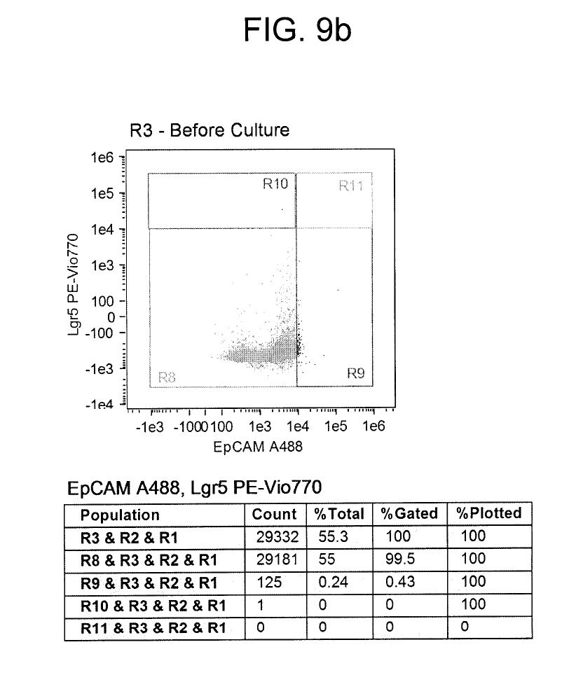

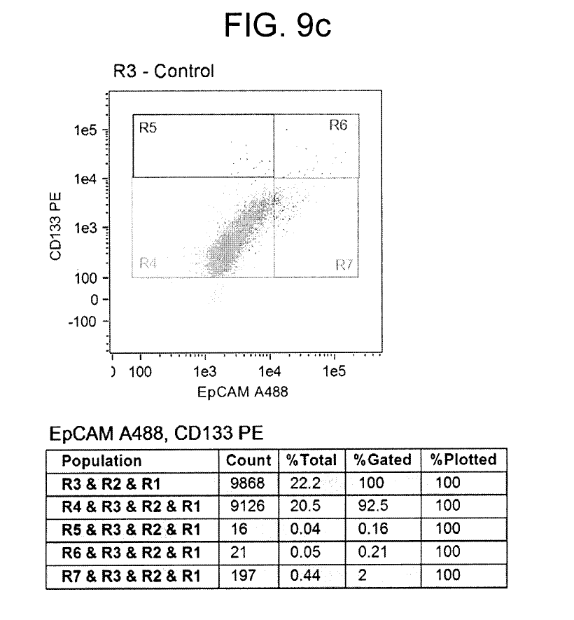

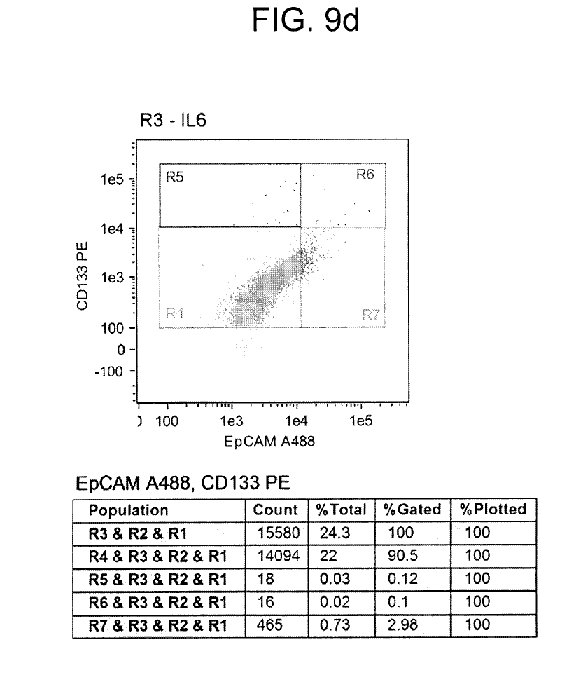

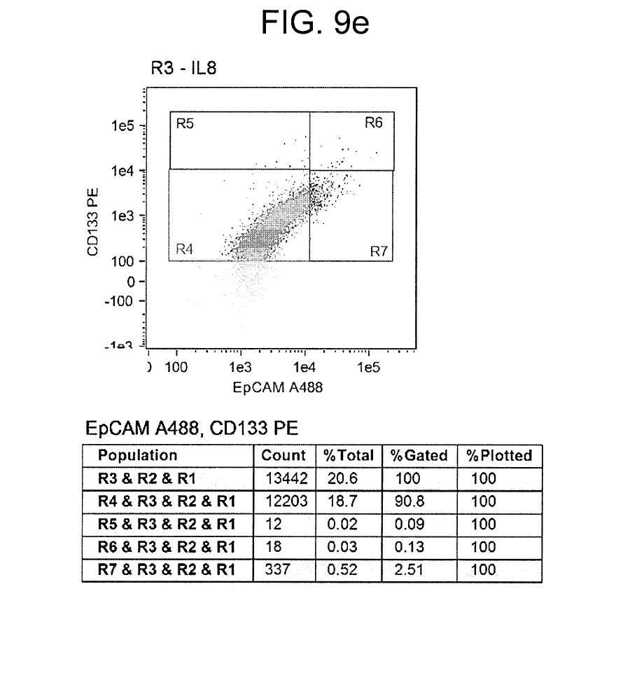

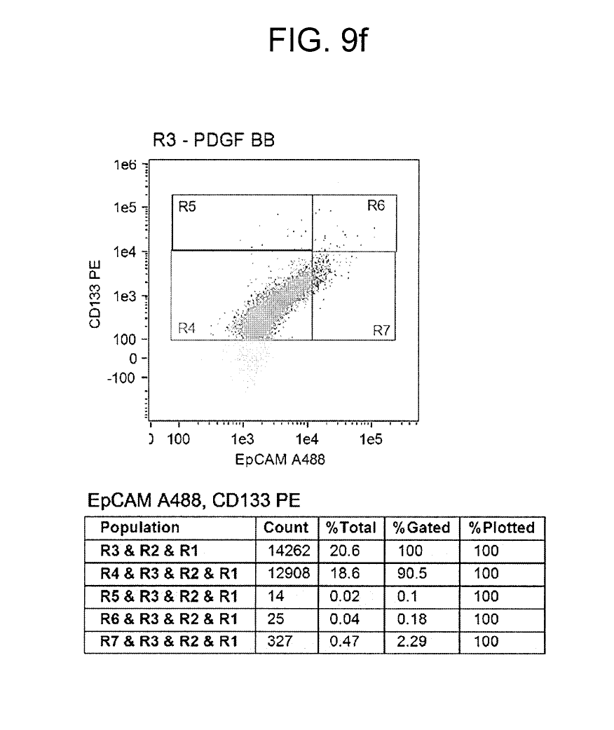

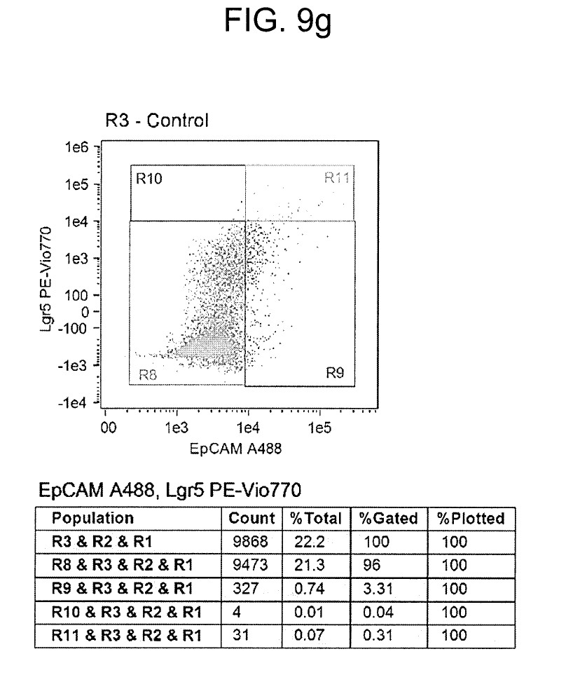

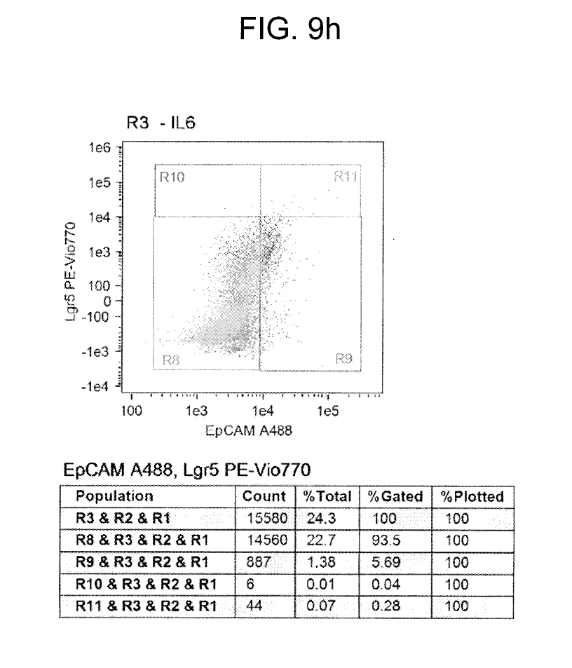

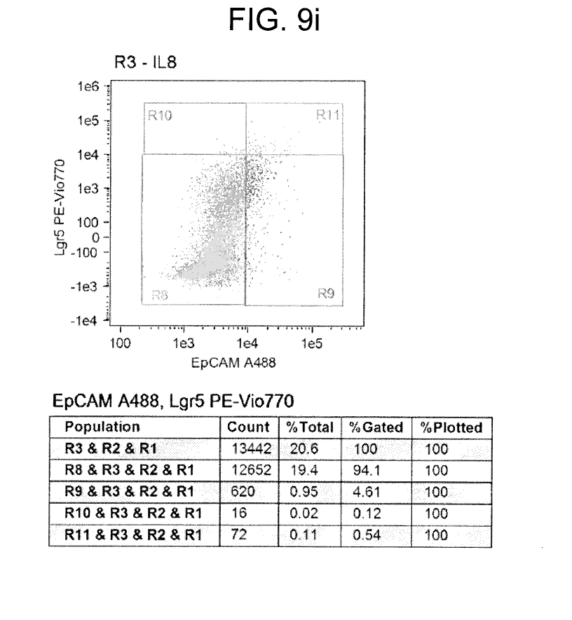

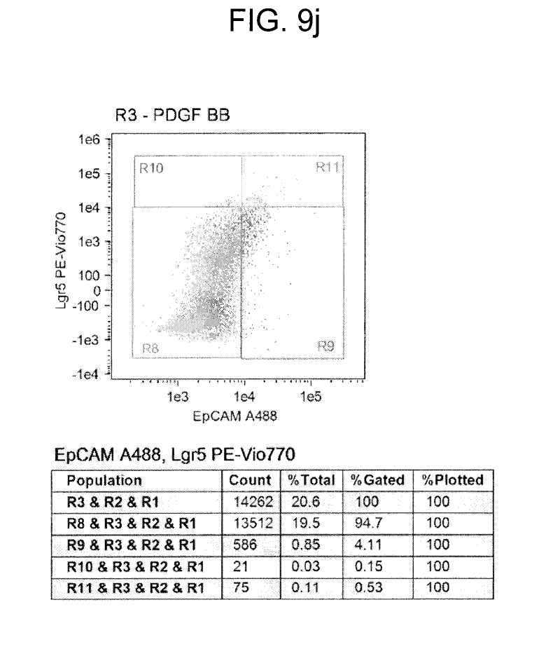

Results show that prior to culture, the subpopulation of EpCam positive cells constituted 99.9% of gated cells. During culture, the subpopulation of EpCAM positive cells dropped to 92.5% in the control cells and 90.5-90.8% in cytokine treated cells as shown, in Table 27. FIG. 8 shows the effect of three cytokines (IL-6, IL-8 and PDGFBB) on enriched CTCs. The cancer stem cell fraction (the EpCAM+CD133− subpopulation) increased from 0.05% before culture to 2% of gated cells in the control and 2.29-2.98% of gated cells in cytokine treated cells (See Table 27) This increase corresponds to more than an order of magnitude. The increase in the EpCAM+CD133− subpopulation following the addition of IL-6 was statistically significant (p<0.05), Additionally, the EpCAM+CD133+ subpopulation is observed at higher percentages in control cells than in cytokine treated cells. This subpopulation is absent in cells before culture (Table 27). This subpopulation may act as an intermediate subpopulation before the cells advance to a full cancer stem cell subpopulation (EpCAM+CD133−). FIG. 9 depicts flow cytometry scatterplots of cells stained with EpCAM A488, CD133PE and Lgr5-PE-Vio770 following treatment with IL-6, IL-8 and PDGFBB.

Flow cytometry data was analysed using IDEAS software. Double events, out of focus events, CD45PercPCy5.5 positive cells, dead cells and non-nucleated events were eliminated from the fluorescence analysis of the cell population. Hoescht 33258 dye was used to stain nuclei of enriched CTCs. All experiments were stained with a fixable live/dead dye eFluor 506 to exclude dead cells.

TABLE 27
Cell populations of cultured circulating tumour cells exhibiting
cancer stem cell marker after treatment with different cytokines.

| | EpCAM−CD133− | EpCAM−CD133+ | EpCAM+CD133+ | EpCAM+CD133− |
|---|---|---|---|---|
| Before Culture | 99.9 | 0.01 | 0 | 0.05 |
| Control | 92.5 | 0.16 | 0.21 | 2 |
| IL-6 | 90.5 | 0.12 | 0.1 | 2.98 |
| Il-8 | 90.8 | 0.09 | 0.13 | 2.51 |
| PDGFBB | 90.5 | 0.1 | 0.18 | 2.29 |

Numbers represent percentages of gated subpopulations. An antibody cocktail with EpCAM A488, CD133 PE and CD45 PerCPCy5.5 was used. No cell stained positive for CD45 PerCPCy5.5.

### Example 5

### Applying Distinct Cytokine Cocktails to Cancer Cells Will Increase Cell Proliferation and EMT

Preliminary data also exists to suggest that applying anti-cancer therapies to cancer cell populations rapidly after exposure to HGF in isolation can improve therapeutic effectiveness against cancer stem cells. This is believed to be caused by this cytokine triggering cellular proliferation in both non-stem and stem cell populations and rendering these dividing cells more vulnerable to anti-cancer therapies during a narrow window of time. This effect appeared to be limited to HGF alone as IL-6, PGE-2, and TGF-beta exposure enriched Irinotecan treated cells for cancer stem cells above non-cytokine treated controls. However, delaying therapy too long after exposure to these cytokines renders most conventional anti-cancer therapies less effective, presumably because many of these cells will have transitioned to the stem cell phenotype and shifted back into dormancy.

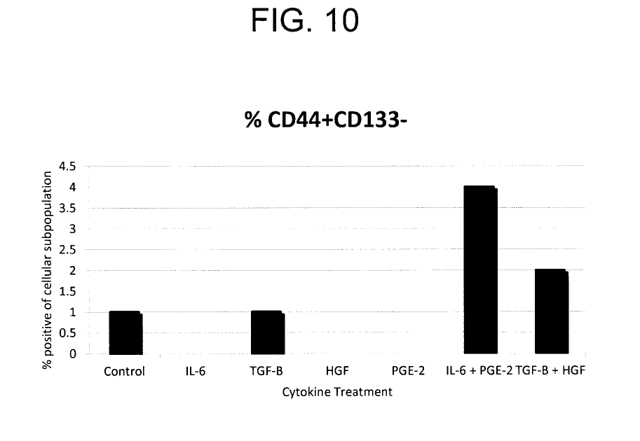

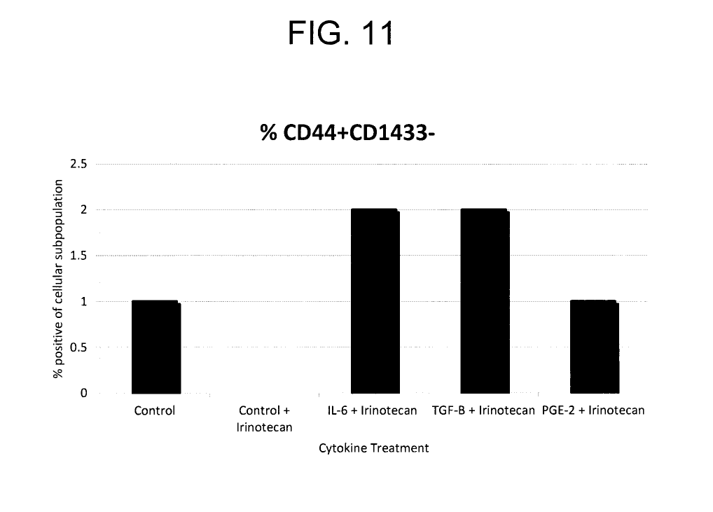

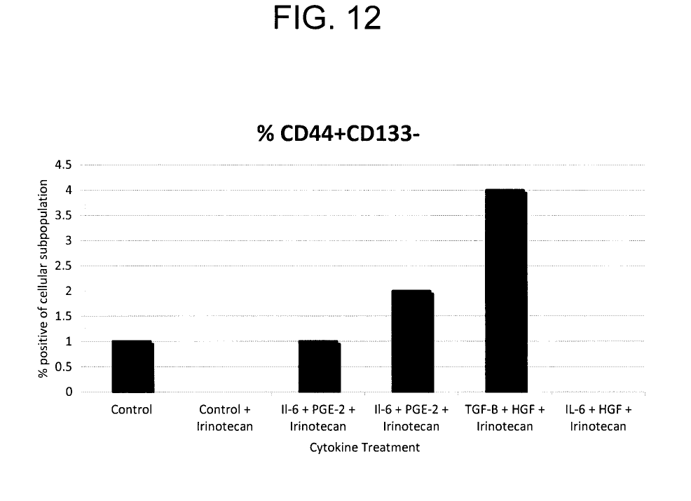

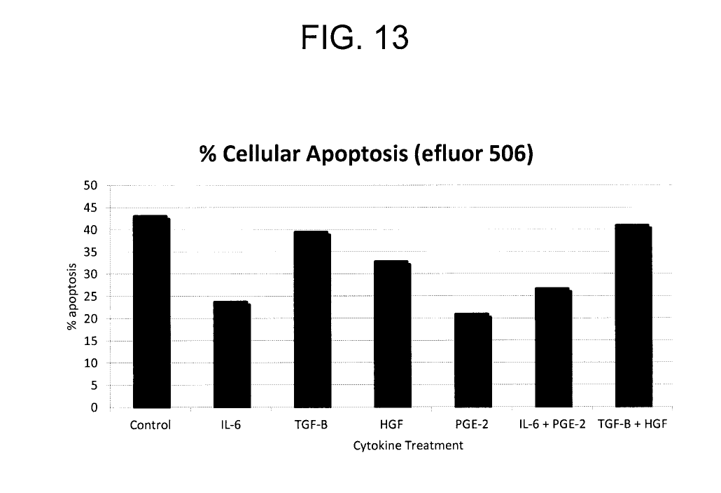

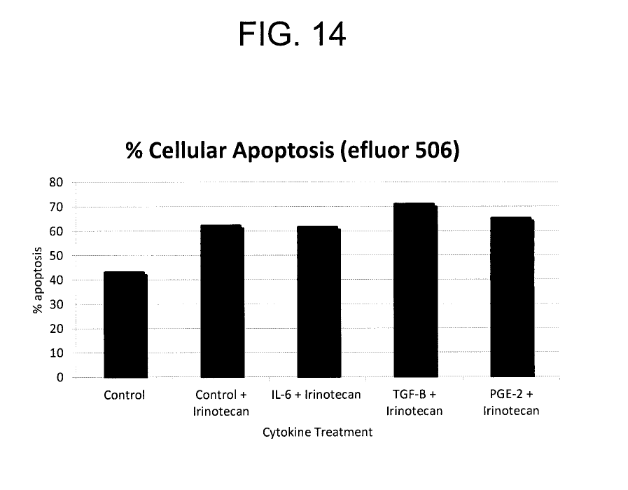

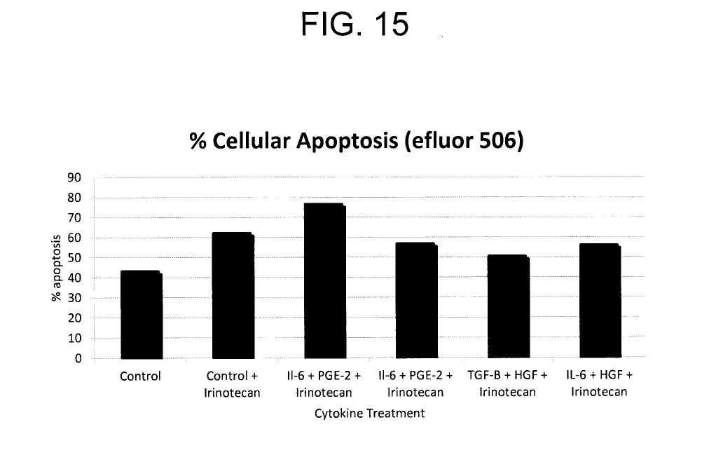

SW-620 cells, a human colorectal adenocarcinoma cell line, were treated with cytokines alone or in combination including IL-6, TGF-beta, HGF, PGE-2, IL-6+PGE-2 and TGF-beta+HGF for 24 hours and compared to untreated SW-620 cells as control, Some treated and untreated SW-620 cells were then exposed to 2 μM Irinotecan for 36 hours at which point cells were harvested for flow cytometry to assess for stem cell markers and markers of apoptosis. The percentage of CD44+CD133− cells following treatment with various cytokine and cytokine cocktails is depicted in FIG. 10. Compared to non-cytokine treated cells, combinations with IL-6+PGE-2, and TGF-beta+HGF increased the number and percentage of cells that were CD44+CD133− as shown in FIG. 10 (stem cells). FIG. 11 depicts the percentage of CD44+CD133− cells following treatment with various cytokines and Irinotecan. Treatment with IL-6+Irinotecan and TGF+Irinotecan increased the percentage of CD44+CD133− stem cells to 2% (from zero in non-treated control cells) as shown in FIG. 11. FIG. 12 depicts the percentage of CD44+CD133− cells following treatment with various cytokine cocktails and Irinotecan. A combination of TGF+HGF+Irinotecan increased the percentage of CD44+CD133− stem cells to 2% (from zero in non-treated control cells) as shown in FIG. 12. Cells treated with HGF+Irinotecan resulted in a log kill ratio of 100% (no viable cells after treatment). Additionally, treatment with IL-6 and PGE-2 appears to have a cytoprotective effective as evidenced by an increased percentage of stem cells that survived treatment as shown in FIG. 13. The effect of treatment of various cytokines and Irinotecan on cellular apoptosis is depicted in FIG. 14, The effect of treatment of various cytokine cocktails and Irinotecan on cellular apoptosis is depicted in FIG. 15.

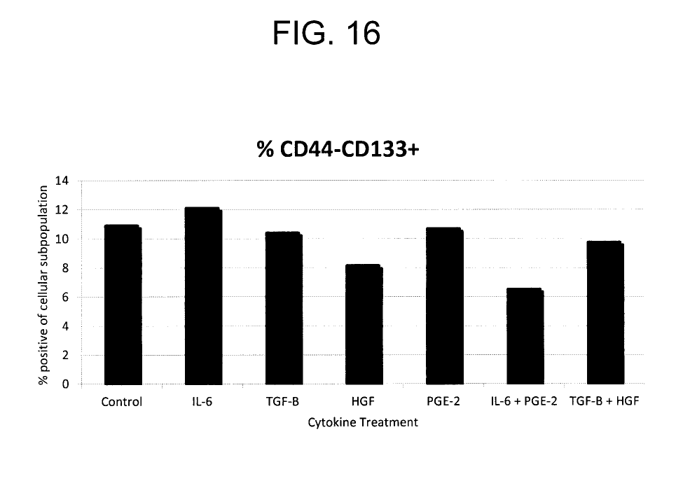

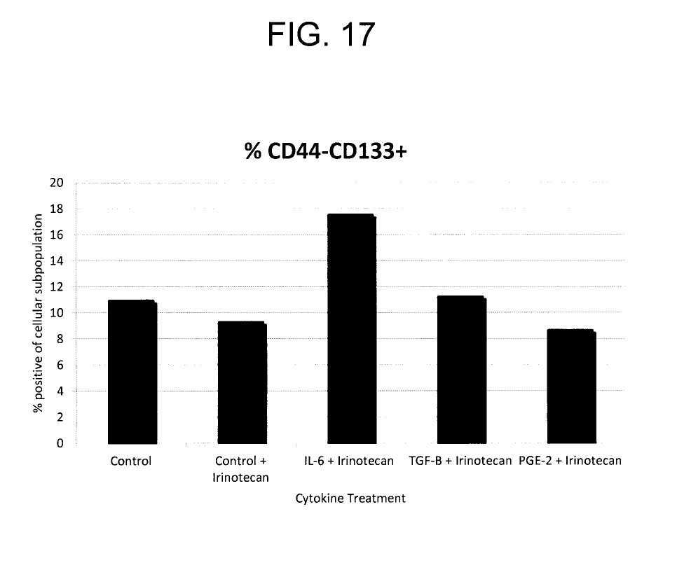

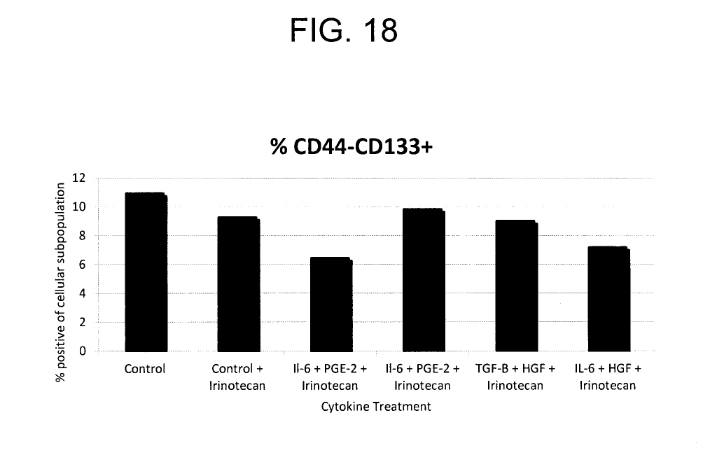

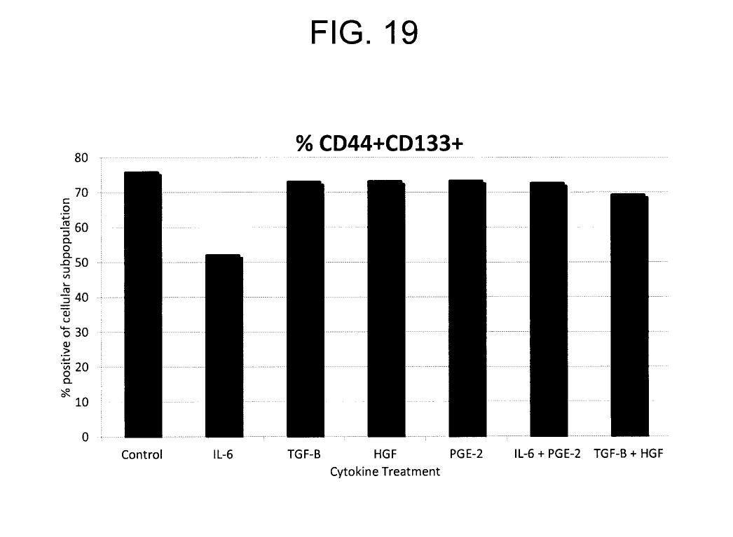

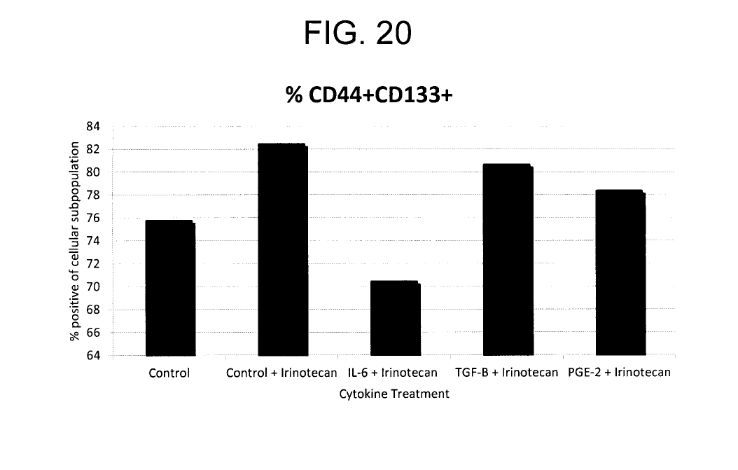

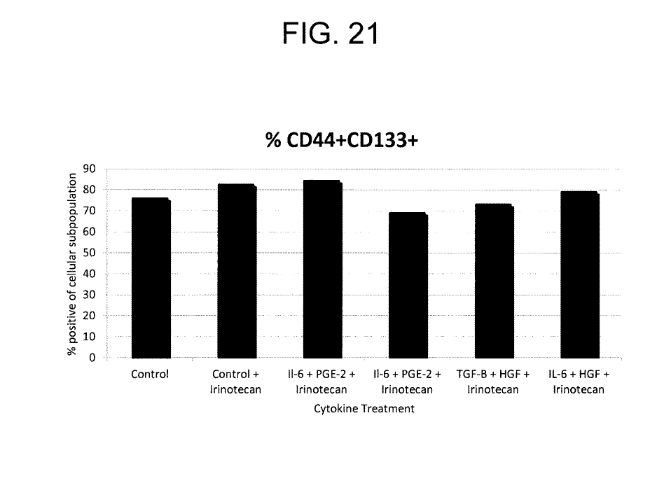

Additionally, the percentage of CD44−CD133+ cells following treatment with various cytokines and cytokines cocktails was determined as depicted in FIG. 16. The percentage of CD44−CD133+ cells following treatment with various cytokines and Irinotecan was measured as depicted in FIG. 17. The percentage of CD44−CD133+ cells following treatment with various cytokine cocktails and Irinotecan was measured as depicted in FIG. 18. The percentage of CD44+CD133+ cells following treatment with various cytokine and cytokine cocktails is depicted in FIG. 19. The percentage of CD44+CD133+ cells following treatment with various cytokines and Irinotecan is depicted in FIG. 20. The percentage of CD44+CD133+ cells following treatment with various cytokine cocktails and Irinotecan is depicted in FIG. 21.

### The Effects of HGF on Chemotherapy

Given the protective nature for stem cell survival afforded by IL-6, PGE-2, and TGF-beta after exposure to chemotherapy, and potentially PDGF-BB given its role in fostering stem cell enrichment as shown in Table 27, and given that HGF in isolation rendered cancer cells highly sensitive to the effects of chemotherapy as detailed above, it is conceptualized that by blocking the influence of these specific cytokines, including IL-6, PGE-2, PGF, TGF-beta, PDGF-BB, MCP-1 and MMP-9 at the time they are upregulated (ligand/receptor/downstream actors) while allowing the predicted upregulation of HGF to occur after tumor removal, a surviving cancer cell population may become very vulnerable to the effects of cytotoxic chemotherapy, including a surviving cancer stem cell population. The chemotherapeutic agents that would work most effectively during this time would be those agents that target the machinery of cell division, given the effect of HGF in isolation triggering cell proliferation and hence vulnerability to agents that disrupt the fidelity of cell division.

### Example 6

The Distinct Cytokine Response after Chemotherapy and Radiation

Given the highly conserved nature of a wound healing response, it is conceptualized that a similar tissue (tumour) repair response will be triggered not only by surgery, but also by chemotherapy and radiation therapy. By blocking this response at the time it is predicted to be upregulated one should also be able to prevent the emergence of a stem cell enriched residual cancer cell population and therefore mitigate the development of a drug resistant phenotype.

The influence of radiation treatment and chemotherapy treatment was studied on patients with either prostate, breast, or colorectal cancers (N=6). Specimens for cytokine testing were collected by aseptic technique into EDTA tubes. Specimens from chemotherapy patients were collected before chemotherapy, 48 hours after chemotherapy and at one week after chemotherapy. Specimens from radiation patients were collected before radiation, and at 24 hours, 48 hours, 72 hours and at one week after radiation.

EDTA samples were centrifuged within 30 minutes of collection, plasma was removed and then recentrifuged. Plasma was then aliquoted into cryotubes and stored in a −80° C. freezer. On the morning of testing, cryotubes containing an aliquot of plasma from designated patients were placed into the 4° C. refrigerator to thaw, then were vortexed and recentrifuged for 5 minutes at 10,000 g. Testing was performed immediately after this.

Results of tests performed using EMD Millipore kits were read on the Luminex 200 analyzer. This flow cytometer based instrument integrates key detection components, such as lasers, optics, advanced fluidics and high speed digital signal processors. The multiplex technology is capable of performing a variety of bioassays including immunoassays on the surface of fluorescent coded magnetic beads known as MagPlex™-C microspheres. Results are quantified based on fluorescent reporter signals.

TGF-beta levels dropped in concert with PDGFBB levels acutely after treatment in 4/6 of the patients. IL-6, HGF and IL-8 levels rose acutely in 4/6 patients after treatment, as did IL-8.

This data represents a preliminary investigation. The results of this study are ongoing and in with future enrollment, the results will be determined longitudinally for longer than one week in duration. However, the present inventor maintains that a distinct inflammatory pattern will emerge in response to both chemotherapy and radiation therapy that can be disrupted to prevent the emergence of a drug resistant surviving cancer cell population. Furthermore, this response can likely be manipulated to block the cytoprotective inflammatory response while given free expression to the proliferative response (HGF) to render these treatments more effective.

### REFERENCES

Balkwill F, Mantovani A: Inflammation and cancer: back to Virchow? Lancet 2001, 357:539-545,
Dvorak H: Tumors: Wounds That Do Not Heal. N Engl J Med 1986, 315:1650-1659.
Kalluri R: EMT: When epithelial cells decide to become mesenchymal-like cells. J Clin Invest 2009, 119:1417-1419.
Thiery J P: Epithelial-mesenchymal transitions in development and pathologies. Curr Opin Cell Biol 2003, 15:740-746.
Cano A, Perez-Moreno M A, Rodrigo I, et al: The transcription factor snail controls epithelial-mesenchymal transitions by repressing E-cadherin expression. Nat Cell Biol 2000, 2:76-83.
Thiery J P, Sleeman J P: Complex networks orchestrate epithelial mesenchymal transitions. Nat Rev Mol Cell Biol 2006, 7:131-142.
Grunert S, Jechlinger M, Beug H: Diverse cellular and molecular mechanisms contribute to epithelial plasticity and metastasis. Nat Rev Mol Cell Biol 2003, 4:657-665.
Boyer B, Dufour S, Thiery J P: E-cadherin expression during the acidic FGF induced dispersion of a rat bladder carcinoma cell line. Exp Cell Res 1992, 201:347-357.
Arnoux V, Come C, Kusewitt D, et al: Cutaneous Wound Reepithelialization: A Partial and Reversible EMT. In Rise and Fall of Epithelial Phenotype: Concepts of Epithelial-Mesenchymal Transition. Edited by: Pierre Savagner. New York, Kluwer Academic; 2005:111-134.
Ahmed N, Maines-Bandiera S, Quinn M, et al: Molecular pathways regulating EGF-induced epithelio-mesenchymal transition in human ovarian surface epithelium. Am J Physiol Cell Physiol 2006, 290:1532-1542.
Broughton G, Janis J, Attinger C: The Basic Science of Wound Healing. Plast Reconstr Surg 2006, 117:12S-33S.
Warren J S, Ward P A, et al: The Inflammatory Response. In Williams Hematology. Edited by: Lichtman M, Beutler E, Kipps T. New York, McGraw Hill; 2006:221-230.
Zavadil J, Bottinger E P: TGF-beta and epithelial-to-mesenchymal transitions. Oncogene 2005, 24:5764-5774.
Tanaka T, Saika S, Ohnishi Y, et al: Fibroblast growth factor 2: roles of regulation of lens cell proliferation and epithelial-mesenchymal transition in response to injury. Mol Vis 2004, 10:462-467.
Nusrat A, Parkos C A, Bacarra A E, et al: Hepatocyte growth factor/scatter factor effects on epithelia. Regulation of intercellular junctions in transformed and nontransformed cell lines, basolateral polarization of cmet receptor in transformed and natural intestinal epithelia, and induction of rapid wound repair in a transformed model epithelium. J Clin Invest 1994, 93:2056-2065.
Jechlinger M, Sommer A, Moriggl R, Seither P, et al: Autocrine PDGFR signaling promotes mammary cancer metastasis, J Clin Invest 2006, 116:15614570.
Pollak M N, Schernhammer E S, Hankinson S E: Insulin-like growth factors and neoplasia, Nat Rev Cancer 2004, 4:505-518.
Slack J M: Stem Cells in Epithelial Tissues. Science 2000, 287:1431-1433.
Amoheslaysky A, Jiang J, Ip Y T: Tissue damage-induced stem cell division in Drosophila. Cell Stem Cell 2009, 4:49-61.
Brown S L, Riehl T E, Walker M R, et al: Myd88-dependent positioning of Ptgs2-expressing stromal cells maintains colonic epithelial proliferation during injury. J Clin Invest 2007, 117:258-269.
Giangreco A, Arwert E N, Rosewell I R, et al: Stem cells are dispensable for lung homeostasis but restore airways after injury. Proc Natl Acad Sci USA 2009, 106:9286-9291.
Li X, Lewis M, Huang J, et al: Intrinsic resistance of tumorigenic breast cancer cells to chemotherapy. J Natl Cancer Inst 2008, 100:672-679.
Dylla S J, Beviglia L, Park In-Kyung, et al: Colorectal cancer stem cells are enriched in xenogeneic tumors following chemotherapy. PLoS ONE 2008, 3:e2428.
Eyler C E, Rich J N: Survival of the fittest: cancer stem cells in therapeutic resistance and angiogenesis. J Clin Oncol 2008, 26:2839-2845,
Early Breast Cancer Trialists' Collaborative Group. Polychemotherapy for early breast cancer: an overview of the randomised trials. The Lancet 352, 930-942 (1998).
N. H. Wolmark et al. The benefit of leucovorin-modulated fluorouracil as postoperative adjuvant therapy for primary colon cancer: results from National Surgical Adjuvant Breast and Bowel Project protocol C-03. J Clin Oncol 11, 1879-1887 (1993).
R. K. Nissen-Meyer, K. Kjellgren, K. Malmio, B. Mansson, T. Norin, Surgical adjuvant chemotherapy: results with one short course with cyclophosphamide after mastectomy for breast cancer. Cancer 41, 2088-2098 (1978).
J. H. Goldie, A. J. Coldman, A mathematic model for relating the drug sensitivity of tumors to their spontaneous mutation rate. Cancer Treat Rep 63, 1727-1733 (1979).
C. E. Eyler, J. N. Rich, Survival of the fittest: cancer stem cells in therapeutic resistance and angiogenesis. J Clin Oncol 26, 2839-2845 (2008).
D. A. Ponti et al. Isolation and in vitro propagation of tumorigenic breast cancer cells with stem/progenitor cell properties. Cancer Res 65, 5506-5511 (2005).
B. Fisher, N. Gunduz, J. Coyle, C. Rudock, E. Saffer, Presence of a growth-stimulating factor in serum following primary tumor removal in mice. Cancer Res 49, 1996-2001 (1989).
F. A. Coumans, S. Siesling, I. W. Terstappen, Detection of cancer before distant metastasis. BMC Cancer 13, 283 (2013).
S. J. Morrison, 3. Kimble, Asymmetric and symmetric stem-cell divisions in development and cancer, Nature 441, 1068-1074 (2006).
C. Chaffer et al. Normal and neoplastic nonstem cells can spontaneously convert to a stem-like state. Proc Natl Acad Sci USA 108, 7950-7955 (2011).
S. A. Mani et al. The epithelial-mesenchymal transition generates cells with properties of stem cells. Cell 133, 704-715 (2008).
P. Savagner, V. Arnoux, Epithelio-mesenchymal transition and cutaneous wound healing. Bull Acad Natl Med 193, 1981-1991 (2009).
W. Harless, Cancer treatments transform residual cancer cell phenotype. Cancer Cell Int. 11, 1 (2011).
M. Al-Hajj, M. S. Wicha, A. Benito-Hernandez, S. J. Morrison, M. F. Clarke, Prospective identification of tumorigenic breast cancer cells. Proc Natl Acad Sci S A 100, 3983-3988 (2003).
M. A. Essers, A. Trumpp, Targeting leukemic stem cells by breaking their dormancy. Mol Oncol 4, 443-450 (2010).
Shuman S: Structure, mechanism, and evolution of the mRNA capping apparatus. Prog. Nucleic Acid Res. Mol. Biol 2000, 66:1-40
Chomczynski P, Sacchi N: Single-step method of RNA isolation by acid guanidinium thiocyanate-phenol-chloroform extraction. Anal Biochem 1987, 162:156-159.
Sambrook J, Fitsch E F, Maniatis T; Molecular Cloning: A Laboratory Manual. Cold Spring Harbor, Cold Spring Harbor Press 1989.
Wessely S, Wood F: Peer review of grant applications: a systematic review. In: Peer review in Health Sciences. Edited by Godlee F, Jefferson T. London, BMJ Books 1999, 14-31.
Advisory Committee on Genetic Modification: Annual Report. London; 1999.
Adams W J, Morris D L (1997) Pilot study—cimetidine enhances lymphocyte infiltration of human colorectal carcinoma: results of a small randomized control trial. Cancer 80 (1): 15-21
Ahern T P, Pedersen L, Tarp M, Cronin-Fenton D P, Game J P, Silliman R A, Sorensen H T, Lash T L, Statin prescriptions and breast cancer recurrence risk: a Danish nationwide prospective cohort study. (2011) J Natl Cancer Inst 103 (19): 1461-1468.
Allegra C J, Yothers G, O'Connell M J, Sharif S, Petrelli N J, Colangelo L H, Atkins J N, Seay T E, Fehrenbacher L, Goldberg R M, O'Reilly S, Chu L, Azar C A, Lopa S, Wolmark N, Phase III trial assessing bevacizumab in stages II and III carcinoma of the colon: results of NSABP protocol C-08. (2011) J Clin Oncol 29 (1): 11-16,
Arnoux V, Come C, Kusewitt D F, Hudson L G, Savagner P, Cutaneous Wound Reepithelialization: A Partial and Reversible EMT. In Rise and Fall of Epithelial Phenotype: Concepts of Epithelial-Mesenchymal Transition, Savagner P (ed) pp 111-134. Kluwer Academic: New York (2005).
Balkwill F (2004) The significance of cancer cell expression of the chemokine receptor CXCR4. Semin Cancer Biol 14 (3): 171-179.
Bendas G, Borsig L Cancer cell adhesion and metastasis: selectins, integrins, and the inhibitory potential of heparins, (2012) Int J Cell Biol 676731.
Berg E L, Magnani J, Warnock R A, Robinson M K, Butcher E C, Comparison of L-selectin and E-selectin ligand specificities: the L-selectin can bind the E-selectin ligands sialyl Le(x) and sialyl Le(a), (1992) Biochem Biophys Res Common 184 (2): 1048-1055
Broughton G, Janis J E, Attinger C E, The basic science of wound healing, (2006) Plast Reconstr Surg 117 (7 Suppl): 12S-34S
Chan A T, Ogino S, Fuchs C S, Aspirin use and survival after diagnosis of colorectal cancer, (2009) JAMA 302 (6): 649-658
Chung A S, Kowanetz M, Wu X, Zhuang G, Ngu H, Finkle D, Komuves L, Peale F, Ferrara N (2012) Differential drug class-specific metastatic effects following treatment with a panel of angiogenesis inhibitors. J Pathol 227 (4): 404-416
Deva S, Jameson M (2012) Histamine type 2 receptor antagonists as adjuvant treatment for resected colorectal cancer. Cochrane Database Syst Rev 8 CD007814
Domingo E, Church D N, Sieber O, Ramamoorthy R, Yanagisawa Y, Johnstone E, Davidson B, Kerr D J, Tomlinson I P, Midgley R (2013) Evaluation of PIK3CA mutation as a predictor of benefit from nonsteroidal anti-inflammatory drug therapy in colorectal cancer. J Clin Oncol 31 (34): 4297-4305
Early Breast Cancer Triallists Collaborative Group (2005) Effects of chemotherapy and hormonal therapy for early breast cancer on recurrence and 15-year survival: an overview of the randomised trials. Lancet 365 (9472): 1687-1717
Erler J T, Bennewith K L, Cox T R, Lang G, Bird D, Koong A, Le Q T, Giaccia A J (2009) Hypoxia-induced lysyl oxidase is a critical mediator of bone marrow cell recruitment to form the premetastatic niche. Cancer Cell 15 (1): 35-44
Essers M A, Trumpp A (2010) Targeting leukemic stem cells by breaking their dormancy. Mol Oncol 4 (5): 443-450
Eyler C E, Rich J N (2008) Survival of the fittest: cancer stem cells in therapeutic resistance and angiogenesis. J Clin Oncol 26 (17): 2839-2845
Fathke C, Wilson L, Hutter J, Kapoor V, Smith A, Hocking A, Isik F (2004) Contribution of bone marrow-derived cells to skin: collagen deposition and wound repair. Stem Cells 22 (5): 812-822
Fisher B, Gunduz N, Coyle J, Rudock C, Saffer E (1989a) Presence of a growth-stimulating factor in serum following primary tumor removal in mice. Cancer Res 49 (8): 1996-2001
Fisher B, Saffer E, Rudock C, Coyle J, Gunduz N (1989b) Effect of local or systemic treatment prior to primary tumor removal on the production and response to a serum growth-stimulating factor in mice. Cancer Res 49 (8): 2002-2004
Folkman J (1971) Tumor angiogenesis: therapeutic implications. N Engl J Med 285 (21): 1182-1186
Forget P, Vandenhende J, Berliere M, Machiels J P, Nussbaum B, Legrand C, De Kock M (2010) Do intraoperative analgesics influence breast cancer recurrence after mastectomy? A retrospective analysis. Anesth Analg 110 (6): 1630-1635
Gazzerro Proto M C, Gangemi G, Malfitano A M, Ciaglia E, Pisanti S, Santoro A, Laezza C, Bifulco M (2012) Pharmacological actions of statins: a critical appraisal in the management of cancer. Pharmacol Rev 64 (1): 102-146
Giangreco A, Arwert E N, Rosewell I R, Snyder J, Watt F M, Stripp B R (2009) Stem cells are dispensable for lung homeostasis but restore airways after injury. Proc Natl Acad Sci USA 106 (23): 9286-9291
Goldie J H, Coldman A J (1986) Theoretical considerations regarding the early use of adjuvant chemotherapy. Recent Results Cancer Res 103 30-35
Hahm K B, Kim W H, Lee S I, Kang J K, Park I S (1995) Comparison of immunomodulative effects of the histamine-2 receptor antagonists cimetidine, ranitidine, and famotidine on peripheral blood mononuclear cells in gastric cancer patients. Scand J Gastroenterol 30 (3): 265-271
Hamalukic M, Huelsenbeck J, Schad A, Wirtz S, Kaina B, Fritz G (2011) Rac1-regulated endothelial radiation response stimulates extravasation and metastasis that can be blocked by HMG-CoA reductase inhibitors. PLoS One 6 (10): e26413
Harless W W (2011) Cancer treatments transform residual cancer cell phenotype. Cancer Cell Int 11 (1): 1
Holmes M D, Chen W Y, Li L, Hertzmark E, Spiegelman D, Hankinson S E (2010) Aspirin intake and survival after breast cancer. J Clin Oncol 28 (9): 1467-1472
Honn K V, Cicone B, Skoff A (1981) Prostacyclin: a potent antimetastatic agent. Science 212 (4500); 1270-1272
Honn K V, Tang D G, Crissman J D (1992) Platelets and cancer metastasis: a causal relationship? Cancer Metastasis Rev 11 (3-4): 325-351
Ikeda M, Furukawa H, Imamura H, Shimizu J, Ishida H, Masutani S, Tatsuta M, Kawasaki T, Satomi T (2002) Surgery for gastric cancer increases plasma levels of vascular endothelial growth factor and von Willebrand factor. Gastric Cancer 5 (3): 137-141
Jin K, Minami M, Lan J Q, Mao X O, Batteur S, Simon R P, Greenberg D A (2001) Neurogenesis in dentate subgranular zone and rostral subventricular zone after focal cerebral ischemia in the rat. Proc Natl Acad Sci USA 98 (8): 4710-4715
Jones M K, Wang H, Peskar B M, Levin E, Itani R M, Sarfeh I J, Tarnawski A S (1999) Inhibition of angiogenesis by nonsteroidal anti-inflammatory drugs: insight into mechanisms and implications for cancer growth and ulcer healing. Nat Med 5 (12): 1418-1423
Kannagi R, Izawa M, Koike T, Miyazaki K, Kimura N (2004) Carbohydrate-mediated cell adhesion in cancer metastasis and angiogenesis. Cancer Sci 95 (5): 377-384
Kaplan R N, Riba R D, Zacharoulis S, Bramley A H, Vincent L, Costa C, MacDonald D D, Jin D K, Shido K, Kerns S A, Zhu Z, Hicklin D, Wu Y, Port J L, Altorki N, Port E R, Ruggero D, Shmelkov S V, Jensen K K, Rafii S, Lyden D (2005) VEGFR1-positive haematopoietic bone marrow progenitors initiate the pre-metastatic niche. Nature 438 (7069); 820-827
Katz M S, Minsky B D, Saltz L B, Riedel E, Chessin D B, Guillem J G (2005) Association of statin use with a pathologic complete response to neoadjuvant chemoradiation for rectal cancer. Int J Radiat Oncol Biol Phys 62 (5): 1363-1370
Khatib A M, Kontogiannea M, Fallavollita L, Jamison B, Meterissian S, Brodt P (1999) Rapid induction of cytokine and E-selectin expression in the liver in response to metastatic tumor cells. Cancer Res 59 (6): 1356-1361
Kim Y S, Bigliani L U, Fujisawa M, Murakami K, Chang S S, Lee H J, Lee F Y, Blaine T A (2006) Stromal cell-derived factor 1 (SDF-1, CXCL12) is increased in subacromial bursitis and downregulated by steroid and nonsteroidal anti-inflammatory agents. J Orthop Res 24 (8): 1756-1764
Kobayashi K, Matsumoto S, Morishima T, Kawabe T, Okamoto T (2000) Cimetidine inhibits cancer cell adhesion to endothelial cells and prevents metastasis by blocking E-selectin expression. Cancer Res 60 (14): 3978-3984
Kollet O, Shivtiel S, Chen Y Q, Suriawinata J, Thung S N, Dabeva M D, Kahn J, Spiegel A, Dar A, Samira S, Goichberg P, Kalinkovich A, Arenzana-Seisdedos F, Nagler A, Hardan I, Revel M, Shafritz D A, Lapidot T (2003) HGF, SDF-1, and MMP-9 are involved in stress-induced human CD34+ stem cell recruitment to the liver. J Clin Invest 112 (2): 160-169
Kollmeier M A, Katz M S, Mak K, Yamada Y, Feder D J, Zhang Z, Jia X, Shi W, Zelefsky M J (2011) Improved biochemical outcomes with statin use in patients with high-risk localized prostate cancer treated with radiotherapy, Int J Radiat Oncol Biol Phys 79 (3): 713-718
Kubota T, Fujiwara H, Ueda Y, Itoh T, Yamashita T, Yoshimura T, Okugawa K, Yamamoto Y, Yano Y, Yamagishi H (2002) Cimetidine modulates the antigen presenting capacity of dendritic cells from colorectal cancer patients. Br J Cancer 86 (8): 1257-1261
Leonardi M J, McGory M L, Ko C Y (2006) The rate of bleeding complications after pharmacologic deep venous thrombosis prophylaxis: a systematic review of 33 randomized controlled trials. Arch Surg 141 (8): 790-797
Li L, Neaves W B (2006) Normal stem cells and cancer stem cells: the niche matters. Cancer Res 66 (9): 4553-4557
Luo D, Renault V M, Rando T A (2005) The regulation of Notch signaling in muscle stem cell activation and postnatal myogenesis. Semin Cell Dev Biol 16 (4-5): 612-622
Ma L, Qiao H, He C, Yang Q, Cheung C H, Kanwar J R, Sun X (2012) Modulating the interaction of CXCR4 and CXCL12 by low-molecular-weight heparin inhibits hepatic metastasis of colon cancer. Invest New Drugs 30 (2): 508-517
Mangano D T (2002) Aspirin and mortality from coronary bypass surgery. N Engl J Med 347 (17); 1309-1317
Mani S A, Guo W, Liao M J, Eaton E N, Ayyanan A, Zhou A Y, Brooks M, Reinhard F, Zhang C C, Shipitsin M, Campbell L L, Polyak K, Brisken C, Yang J, Weinberg R A (2008) The epithelial-mesenchymal transition generates cells with properties of stem cells. Cell 133 (4): 704-715
Marschang P, Friedrich G J, Ditlbacher H, Stoeger A, Nedden D Z, Kirchmair R, Dienstl A, Pachinger O, Patsch J R (2006) Reduction of soluble P-selectin by statins is inversely correlated with the progression of coronary artery disease. Int J Cardiol 106 (2): 183-190
Massberg S, Konrad I, Schurzinger K, Lorenz M, Schneider S, Zohlnhoefer D, Hoppe K, Schiemann M, Kennerknecht E, Sauer S, Schulz C, Kerstan S, Rudelius M, Seidl S, Sorge F, Langer H, Peluso M, Goyal P, Vestweber D, Emambokus N R, Busch D H, Frampton J, Gawaz M (2006) Platelets secrete stromal cell-derived factor 1alpha and recruit bone marrow-derived progenitor cells to arterial thrombi in vivo, 7 Exp Med 203 (5): 1221-1233
Mauti L A, Le Bitoux M A, Baumer K, Stehle J C, Golshayan D, Provero P, Stamenkovic I (2011) Myeloid-derived suppressor cells are implicated in regulating permissiveness for tumor metastasis during mouse gestation. J Clin Invest 121 (7): 2794-2807
Moreira A L, Sampaio E P, Zmuidzinas A, Frindt P, Smith K A, Kaplan G (1993) Thalidomide exerts its inhibitory action on tumor necrosis factor alpha by enhancing mRNA degradation. J Exp Med 177 (6): 1675-1680
Nakamura K, Kariyazono H, Shinkawa T, Yamaguchi T, Yamashita T, Ayukawa O, Moriyama Y, Yotsumoto G, Toyohira H, Taira A, Yamada K (1999) Inhibitory effects of H2-receptor antagonists on platelet function in vitro. Hum Exp Toxicol 18 (8): 487-492
Natori T, Sata M, Nagai R, Makuuchi M (2005) Cimetidine inhibits angiogenesis and suppresses tumor growth. Biomed Pharmacother 59 (1-2): 56-60
Ng K, Ogino S, Meyerhardt J A, Chan J A, Chan A T, Niedzwiecki D, Hollis D, Saltz L B, Mayer R J, Benson A B, III, Schaefer P L, Whittom R, Hartel A, Goldberg R M, Bertagnolli M M, Venook A P, Fuchs C S (2011) Relationship Between Statin Use and Colon Cancer Recurrence and Survival: Results From CALGB 89803. J Natl Cancer Inst
Nierodzik M L, Karpatkin S (2006) Thrombin induces tumor growth, metastasis, and angiogenesis: Evidence for a thrombin-regulated dormant tumor phenotype. Cancer Cell 10 (5): 355-362
Nierodzik M L, Klepfish A, Karpatkin S (1995) Role of platelets, thrombin, integrin IIb-IIIa, fibronectin and von Willebrand factor on tumor adhesion in vitro and metastasis in vivo. Thromb Haemost 74 (1): 282-290
Nissen-Meyer R, Kjellgren K, Malmio K, Mansson B, Norin T (1978) Surgical adjuvant chemotherapy: results with one short course with cyclophosphamide after mastectomy for breast cancer. Cancer 41 (6): 2088-2098
Nubel T, Dippold W, Kleinert H, Kaina B, Fritz G (2004) Lovastatin inhibits Rho-regulated expression of E-selectin by TNFalpha and attenuates tumor cell adhesion. FASEB J 18 (1); 140-142
Ordemann J, Jacobi C A, Schwenk W, Stosslein R, Muller J M (2001) Cellular and humoral inflammatory response after laparoscopic and conventional colorectal resections. Surg Enclose 15 (6): 600-608
Palumbo J S, Talmage K E, Massari J V, La Jeunesse C M, Flick M J, Kombrinck K W, Jirouskova M, Degen J L (2005) Platelets and fibrin(ogen) increase metastatic potential by impeding natural killer cell-mediated elimination of tumor cells. Blood 105 (1): 178-185
Parise L V, Smyth S S, Shet A S, et al (2006) Platelet morphology, biochemistry, and function. In Williams Hematology, Lichtman M, Beutler E, Kipps T, et al (eds) pp 1587-1663. McGraw Hill: New York
Park J H, McMillan D C, Horgan P G, Roxburgh C S (2013) The impact of anti-inflammatory agents on the outcome of patients with colorectal cancer. Cancer Treat Rev
Sceneay J, Parker B S, Smyth M J, Moller A (2013a) Hypoxia-driven immunosuppression contributes to the pre-metastatic niche. Oncoimmunology 2 (1); e22355
Sceneay J, Smyth M J, Moller A (2013b) The pre-metastatic niche: finding common ground. Cancer Metastasis Rev 32 (3-4): 449-464
Schneider M R, Schillinger E, Schirner M, Skuballa W, Sturzebecher S, Witt W (1991) Effects of prostacyclin analogues in in vivo tumor models. Adv Prostaglandin Thromboxane Leukot Res 21B 901-908
Seekamp A, Jochum M, Ziegler M, van Griensven M, Martin M, Regel G (1998) Cytokines and adhesion molecules in elective and accidental trauma-related ischemia/reperfusion. J Trauma 44 (5): 874-882
Shakhar G, Ben Eliyahu S (2003) Potential Prophylactic Measures Against Postoperative Immunosuppression: Could They Reduce Recurrence Rates in Oncological Patients? Annals of Surgical Oncology 10 (8): 972-992
Smorenburg S M, Van Noorden C J (2001) The complex effects of heparins on cancer progression and metastasis in experimental studies. Pharmacol Rev 53 (1): 93-105
Stevenson J L, Varki A, Borsig L (2007) Heparin attenuates metastasis mainly due to inhibition of P- and L-selectin, but non-anticoagulant heparins can have additional effects. Thromb Res 120 Suppl 2 S107-S111
Strachan L, Noble D W (2001) Hypoxia and surgical patients—prevention and treatment of an unnecessary cause of morbidity and mortality. J R Coll Surg Edinb 46 (5): 297-302
Syk I, Hedges S R, Zederfeldt B, Risberg B (1999) Enhanced cytokine response to surgical trauma in patients with colorectal cancer. Int J Surg Investig 1 (3): 167-176
Wong C C, Gilkes D M, Zhang H, Chen 1, Wei H, Chaturvedi P, Fraley S I, Wong C M, Khoo U S, Ng I O, Wirtz D, Semenza G L (2011) Hypoxia-inducible factor 1 is a master regulator of breast cancer metastatic niche formation. Proc Natl Acad Sci USA 108 (39): 16369-16374
Yamashita J I, Kurusu Y, Fujino N, Saisyoji T, Ogawa M (2000) Detection of circulating tumor cells in patients with non-small cell lung cancer undergoing lobectomy by video-assisted thoracic surgery: a potential hazard for intraoperative hematogenous tumor cell dissemination. J Thorac Cardiovasc Surg 119 (5): 899-905.
Al-Hajj M A, Wicha M S, Benito-Hernandez A, Morrison S J, Clarke M F, Prospective identification of tumorigenic breast cancer cells. Proc Natl Acad Sci USA 2003, 100:3983-3988
Eyler C E, Rich J N. Survival of the fittest: cancer stem cells in therapeutic resistance and angiogenesis. J Clin Oncol 2008, 26:2839-2845
Essers M A, Trumpp A. Targeting leukemic stem cells by breaking their dormancy. Mol Oncol 2010, 4:443-450
Giangreco A, Arwert E N, Rosewell I R, Snyder J, Watt F M, Stripp B R. Stem cells are dispensable for lung homeostasis but restore airways after injury. Prac Natl Acad Sci USA 2009, 106:9286-91
Jin K, Minami M, Lan J Q et al, Neurogenesis in dentate subgranular zone and rostral subventricular zone after focal cerebral ischemia in the rat. Proc Natl Read Sci USA 2001, 98: 4710-4715
Fisher B, Gunduz N, Coyle J, Rudock C, Saffer E. Presence of a growth stimulating factor in serum following primary tumor removal in mice. Cancer Res (1989), 49:1996-2001
Savagner P, Arnoux V. Epithelio-mesenchymal transition and cutaneous wound healing. Bull Acad Natl Med (2009), 193:1981-1991
Mani S A, et al. The epithelial-mesenchymal transition generates cells with properties of stem cells. Cell (2008), 133:704-715
Harless W. Cancer treatments transform residual cancer cell phenotype. Cancer Cell Int. 2011, 11:1
Abubaker K, et al. Short term single treatment of chemotherapy results in the enrichment of ovarian cancer stem cell like cells and increased tumor burden. Molecular Cancer 2013, 12:24
Printz C. Radiation treatment generates therapy-resistant cancer stem cells from less aggressive breast cancer cells. Cancer 2012, 118:3225-33.

What is claimed is:

1. A method for preventing or inhibiting metastasis and/or recurrence of a cancer and/or drug resistance in a patient after a primary treatment of the patient, the method comprising:
(a) administering a therapeutically effective amount of a compound or composition for inhibiting stem cell enrichment in any surviving cancer cell population.

2. The method of claim 1, wherein inhibiting stem cell enrichment comprises inhibiting at least one of: cancer stem cell self-renewal and induction of epithelial-mesenchymal transition in a cancer cell.

3. The method of claim 2, wherein the composition is administered perioperatively.

4. The method of claim 3 wherein the compound or composition is administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

5. The method of claim 4, wherein the primary treatment is one of endocrine therapy, chemotherapy, radiotherapy, hormone therapy, surgery, gene therapy, thermal therapy, and ultrasound therapy.

6. The method of claim 5, wherein the primary treatment is excision of a solid tumor.

7. The method of claim 5 or 6, wherein the compound or composition comprises a non-steroidal anti-inflammatory drug, a heparin, cytotoxic chemotherapy or a cytokine inhibitor.

8. The method of claim 7, wherein the compound or composition comprises a cytokine inhibitor.

9. The method of claim 8, wherein the compound or composition comprises one or more antibodies specific for at least one cytokine involved in stem cell enrichment.

10. The method of claim 8 or 9, wherein the cytokine comprises at least one of: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MMP-9, PDGF-BB and PGF; and more preferably all of HGF, IL-6, TGF-beta, PGE-2, PDGF-BB.

11. The method of claim 8, wherein the compound or composition inhibits the upregulation of the one or more cytokines for at least about 48, at least about 72, at least about 96, or at least about 120 hours post primary treatment.

12. The method of claim 5 or 6, wherein the compound or composition comprises a neu-1 sialidase inhibitor, preferably oseltamivir phosphate.

13. The method of any one of claims 1-12, wherein the composition further comprises a therapeutically effective amount of a further therapeutic agent selected from: a non-steroidal anti-inflammatory drug, a heparin and cytotoxic chemotherapy.

14. A method for treating cancer in a patient, the method comprising:
(a) administering a primary treatment to the patient; and
(b) administering a compound or composition for inhibiting stem cell enrichment in any surviving cancer cell population;
wherein the compound or composition is administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

15. A pharmaceutical composition for preventing or inhibiting metastasis or recurrence of a cancer or drug resistance in a patient after a primary treatment of the cancer, the composition comprising a pharmaceutically acceptable carrier and a therapeutically effective amount of an inhibitor of at least one cytokine associated with stem cell enrichment.

16. The composition of claim 15, wherein the inhibitor is an antibody specific for the at least one cytokine associated with stem cell enrichment.

17. The composition of claim 15 or 16, wherein the cytokine(s) are selected from: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

18. The composition of claim 17, comprising a further therapeutic agent selected from: a non-steroidal anti-inflammatory drug, a heparin and cytotoxic chemotherapy.

19. The composition of any one of claims 15-18, wherein the composition is administered perioperatively.

20. The composition of claim 19 wherein the composition is administered prior to 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or prior to 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 or 0.1 hour(s) after the primary treatment, or immediately after the primary treatment, or prior to the primary treatment.

21. A prophylactic method of inhibiting a risk of metastasis or recurrence of a cancer or drug resistance in a patient diagnosed with the cancer, the method comprising:
(a) administering a therapeutically effective amount of a composition for inhibiting stem cell enrichment.

22. The method of claim 21, wherein the composition is administered prior to a primary treatment.

23. The method of claim 21 or 22, wherein the primary treatment is one of endocrine therapy, chemotherapy, radiotherapy, hormone therapy, surgery, gene therapy, thermal therapy, and ultrasound therapy.

24. The method of any one of claims 21-23, wherein the composition comprises an inhibitor of at least one cytokine associated with stem cell enrichment.

25. The method of claim 24, wherein the cytokine comprises at least one of: TGF-beta, HGF, IL-6, PGE-2, MCP-1, MMP-9, PDGF-BB and, PGF; preferably at least two of TGF-beta, HGF, IL-6, PGE-2, MCP-1, MMP-9, PDGF-BB and PGF; preferably HGF and IL-6 and optionally one or more of TGF-beta, PGE-2, MCP-1, MMP-9, PDGF-BB and PGF; and more preferably all of HGF, IL-6, TGF-beta, PGE-2, PDGF-BB.

26. The method of any one of claims 21-25, wherein the composition comprises a therapeutically effective amount of a therapeutic agent selected from: a non-steroidal anti-inflammatory drug, a heparin and cytotoxic chemotherapy.

27. A method of determining whether a patient is at risk of metastasis or recurrence of a cancer after primary treatment of the cancer, the method comprising:
determining a level of at least one cytokine associated with stem cell enrichment in a sample of the patient after the primary treatment;
wherein a higher level of the at least one cytokine correlates to a higher risk of recurrence.

28. The method of claim 27, wherein the cytokine(s) are selected from: TGF-beta, HGF, IL-6, PGE-2, MCP-1, MMP-9, PDGF-BB, and PGF, preferably HGF, IL-6, TGF-beta, PGE-2, PDGF-BB.

29. The method of claim 28, wherein the level is determined at least one of 30, 29, 28, 27, 26, 25, 24, 23, 22, 21, 20, 19, 18, 17, 16, 15, 14, 13, 12, 11, 10, 9, 8, 7, 6, 5, 4, 3, 2 and 1 day(s) after the primary treatment, and 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.3, 0.2 and 0.1 hour(s) after the primary treatment, and immediately after the primary treatment, and prior to the primary treatment.

30. The method of any one of claims 27-29, wherein the level is determined in a patient sample, and wherein the sample is selected from the group consisting of: blood sample, serum sample, tissue sample and tumour sample.

31. The method of claim 30, wherein level is determined by mRNA or protein level analysis.

32. A kit for use in a method according to any one of claims 27-31, the kit comprising: one or more antibodies specific for a cytokine associated with stem cell enrichment.

33. A method of determining a risk of metastasis or recurrence associated with a cancer in a patient after a primary therapy, the method comprising:
(a) determining activity levels associated with at least one cytokine involved in stem cell enrichment in a sample of the patient after the primary therapy;
(b) constructing an activity profile of the patient from the activity levels;
(c) comparing the activity profile to a reference activity profile with a predetermined risk of metastasis or recurrence; and
wherein if the activity profile has a value greater than that of the reference activity profile, then the risk of metastasis or recurrence is greater than the reference activity profile, and if the activity profile has a value less than that of the reference activity profile, then the risk of metastasis or recurrence is lower than the reference activity profile.

34. The method of claim 33, wherein the activity levels are determined by mRNA level or protein level analysis.

35. The method of claim 33, wherein the at least one cytokine is selected from the group consisting of: TGF-beta, HGF, PGE-2, PGF, PDGF-BB, MCP-1 and MMP-9.

36. The method of claim 35, comprising determining the activity levels of HGF and IL-6, preferably HGF, IL-6, TGF-beta, PDGF-BB, PGE-2.

37. The method of any one of claims 33-36, Wherein the reference activity profile is that of a patient who does not have metastasis or recurrence of the cancer a predetermined period of time after primary surgery.

38. The method of any one of claims 33-35, wherein the reference activity profile is that of the patient prior to primary treatment.

39. The method of any one of claims 33-38, wherein the activity levels are determined at one or more of 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day(s) after the primary treatment, or 24, 23, 22, 21, 20, 19, 18 17 16, 15, 14, 13, 12, 11, 10, 9, 8, 7, 6, 5, 4, 3, 2, 1, 0.9, 0.8, 0.7, 0.6, 0.5, 0.4, 0.5, 0.2 or 0.1 hour(s) after the primary treatment, or immediately after the primary treatment, or prior to the primary treatment.

40. The method of any one of claims 33-39, wherein the sample is one of a blood sample, tumour sample, serum sample and tissue sample of the patient.

41. A method for preventing or inhibiting metastasis or recurrence of a cancer or drug resistance in a patient after a primary treatment of the patient, the method comprising:
(a) interfering with the cellular repair mechanisms invoked by the cancer cells after the primary treatment.

42. A method of preventing or inhibiting metastasis or recurrence of a cancer or drug resistance in a patient after a primary treatment of the patient comprising:
(a) administering a therapeutically effective amount of a cancer therapy targeted towards a population of proliferating cancer stem cells in the patient.

43. A method of treating a cancer in a patient comprising:
(a) administering a treatment that induces a population of cancer stems cells in the patient to proliferate; and
(b) administering a therapeutically effective amount of a cancer therapy targeted towards the proliferating cancer stem cells in the patient.

44. A method of inhibiting metastasis or recurrence of a cancer in a patient after a primary treatment of the patient comprising:
(a) obtaining a patient sample;
(b) determining a cancer stem cell proliferation profile for the patient;
(c) comparing the cancer stem cell proliferation profile to a reference cancer stem cell proliferation profile; and
(d) administering a therapeutically effective amount of a cancer therapy targeted towards a population of proliferating cancer stem cells in the patient if the cancer stem cell proliferation profile is greater than or equal to the reference cancer stem cell proliferation profile.

45. The method of claim 44, wherein determining the cancer stem cell proliferation profile comprises determining a concentration of markers in the patient sample indicative of cancer stem cell proliferation.

46. The method of claim 44, wherein determining the cancer stem cell proliferation profile comprises determining the rate of proliferation of cancer stem cells in the patient sample.

47. The method of claim 44, wherein the patient sample is obtained at one or more of the group consisting of: prior to primary treatment, 1 hour after primary treatment, within 6 hours after primary treatment, within 12 hours after primary treatment, within 18 hours after primary treatment, within 24 hours after primary treatment, within 30 hours after primary treatment, within 36 hours after primary treatment, within 42 hours after primary treatment, within 48 hours after primary treatment, within 54 hours after primary treatment, within 60 hours after primary treatment, within 66 hours after primary treatment, within 72 hours after primary treatment, within 78 hours after primary treatment, within 84 hours after primary treatment, within 90 hours after primary treatment and within 96 hours after primary treatment.

48. The method of any one of claims 44 to 47, wherein the cancer therapy is administered when the cancer stem cell proliferation profile is greater than or equal to the reference cancer stem cell proliferation profile.

49. The method of any one of claims 42 to 48, wherein the cancer therapy is administered within: 6 hours, 12 hours, 18 hours, 24 hours, 30 hours, 36 hours, 42 hours, 48 hours, 54 hours, 60 hours, 66 hours, 72 hours, 78 hours, 84 hours, 90 hours or 96 hours, after administering a treatment that induces a population of cancer stems cells in the patient to proliferate or after primary treatment.

50. The method of claim 49, wherein the cancer therapy may be one or more of the group consisting of: endocrine therapy, chemotherapy, hormone therapy, gene therapy, thermal therapy, ultrasound therapy and immunotherapy.

51. The method of claim 50, wherein the cancer therapy comprises nanoparticle-mediated thermal therapy.

52. The method of claim 50, wherein the cancer therapy is cytotoxic chemotherapy.
cytotoxic chemotherapy is selected from alkylators (including cyclophosphamide/cisplatin/melphalan); topoisomerase inhibitors 1 and 2 (including doxorubicin/irinotecan/etoposide/topotecan); taxanes (including docetaxel/paclitaxel/abraxane); vinca alkaloids (including vincristine/vinblastine); and antimetabolites (including 5-FU/Gemcitabine/Cytarabine/Pemetrexed).

53. The method of any one of claims 42 to 52, wherein administering the cancer therapy targeted towards the population of proliferating cancer stem cells inhibits proliferation of the population of cancer stein cells or induces apoptosis in the population of cancer stem cells.

54. A method of treating cancer in a patient comprising:
perioperatively administering an inhibitor of one or more cytokines selected from: TGF-β, IL-6, MCP-1, PDGF-BB, MMP-9 and PGF, wherein an inhibitor of HGF is not administered;
and perioperatively administering cytotoxic chemotherapy targeted to proliferating cancer cells.

55. The method of claim 54, wherein the cytotoxic chemotherapy is selected from alkylators (including cyclophosphamide/cisplatin/melphalan); topoisomerase inhibitors 1 and 2 (including doxorubicin/irinotecan/etoposide/topotecan); taxanes (including docetaxel/paclitaxel/abraxane); vinca alkaloids (including vincristine/vinblastine); and antimetabolites (including 5-FU/Gemcitabine/Cytarabine/Pemetrexed).

56. The method of claim 54, wherein cytotoxic chemotherapy and/or cytokine inhibitor are administered within: 6 hours, 12 hours, 18 hours, 24 hours, 30 hours, 36 hours, 42 hours, 48 hours, 54 hours, 60 hours, 66 hours, 72 hours, 78 hours, 84 hours, 90 hours or 96 hours after surgery to remove a solid tumour.
